(19) United States
(12) Patent Application Publication
(10) Pub. No.: US 2017/0152269 A1
(43) Pub. Date: Jun. 1, 2017

(54) FUSED BICYCLIC COMPOUNDS AND THEIR USE AS CDK INHIBITORS

(71) Applicants: Jin Li, Nanjing (CN); Liu Liu, Ann Arbor, MI (US); Minmin Yang, Nanjing (CN)

(72) Inventors: Jin Li, Nanjing (CN); Liu Liu, Ann Arbor, MI (US); Minmin Yang, Nanjing (CN)

(73) Assignee: ANONCO SCIENCES, INC., Ann Arbor, MI (US)

(21) Appl. No.: 15/430,752

(22) Filed: Feb. 13, 2017

(30) Foreign Application Priority Data
Feb. 16, 2016 (CN) 201610087998.2

(51) Int. Cl. C07D 495/04

(52) U.S. Cl. CPC C07D 495/04

(57) ABSTRACT

The present invention provides a fused bicyclic compound according to formula (H) or a pharmaceutically acceptable salt thereof, wherein L is —S— or —O—; M is —CH═, —N═ or —CJ=, wherein J is a non-hydrogen monovalent group; and W, G⁰ and G¹ are independently of each other a non-hydrogen monovalent group. The invention further provides a pharmaceutical composition comprising the compound, a method of synthesizing the compound, a method for inhibiting CDK(s), a method for treating a disease or disorder in a mammal mediated by or associated with cyclin-dependent kinase (CDK), and a use of the compound in the manufacturing of a medicament for treating a CDK-mediated disease or disorder.

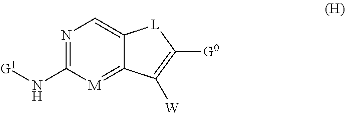

### CROSS-REFERENCE TO RELATED APPLICATIONS

This non-provisional application expressly claims the benefit of priority under the Paris Convention based on Chinese Application No. 201610087998.2 filed on Feb. 16, 2016, the entire disclosures of which is incorporated herein by reference.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### FIELD OF THE INVENTION

The present invention generally relates to fused bicyclic compounds such as thieno[2,3-c]pyridine derivatives and pharmaceutically acceptable salts thereof, processes for the preparation of such compounds, intermediates used in the preparation of such compounds, compositions containing such compounds or salts, and their uses for treating CDK-mediated or CDK-associated diseases and disorders including, e.g., cancer, and autoimmune diseases.

### BACKGROUND OF THE INVENTION

As a species of phosphotransferase, kinase enzymatically catalyzes phosphorylation, in which phosphate groups are transferred from phosphate-donating molecules to substrates. This transesterification reaction produces a phosphorylated substrate and ADP. The phosphorylation may be triggered in response to a variety of stimuli, such as osmotic shock, heat shock, ultraviolet radiation, bacterial endotoxin, H₂O₂, cytokines including interleukin-1 (IL-1) and tumor necrosis factor-α (TNF-α), growth factors such as granulocyte macrophage-colony-stimulating factor (GM-CSF), and fibroblast growth factor (FGF). An extracellular stimulus may affect one or more cellular responses related to cell growth, migration, differentiation, secretion of hormones, activation of transcription factors, muscle contraction, glucose metabolism, control of protein synthesis, and regulation of the cell cycle. The phosphorylation state of a molecule (e.g. a protein, lipid, or carbohydrate) has an impact on its activity, reactivity, and its ability to bind other molecules. As such, kinases are critically important in metabolism, cell signaling, protein regulation, cellular transport, secretory processes, and many other cellular pathways. For example, many diseases are associated with abnormal cellular responses triggered by protein kinase-mediated events. These diseases include, but are not limited to, cancer, autoimmune diseases, inflammatory diseases, bone diseases, metabolic diseases, neurological and neurodegenerative diseases, cardiovascular diseases, allergies and asthma, and hormone-related diseases.

Because phosphorylation is a reversible covalent modification, phosphorylation of a substrate such as protein is regulatory. These phosphorylation events function as molecular on/off switches that can modulate or regulate the target substrate's biological function. As such, there has been a great effort in medicinal chemistry to find kinase inhibitors, especially protein kinase inhibitors, which are effective as therapeutic agents.

Protein kinases may phosphorylate proteins on their serine, threonine, tyrosine, or histidine residues, and thereby increase or decrease the protein's activity, stabilize it, mark it for destruction, localize it within a specific cellular compartment, and initiate or disrupt its interaction with other proteins. Cyclin-dependent kinases (CDKs) are a family of protein kinases that can regulate the cell cycle, signal transduction processes within the cell, transcription, mRNA processing, and the differentiation of nerve cells. A CDK must bind to a regulatory protein called a cyclin, because only the CDK-cyclin complex is an active kinase. The CDK family comprises 21 subtypes which function through the interactions with different regulatory cyclin subunits. Besides their regulation on cell cycles, different subtypes of CDK also regulate transcription, DNA repair, differentiation, and apoptosis. Known CDK-cyclin complexes include CDK1-Cyclin B regulating M phase, CDK1-Cyclin A regulating G2 phase, CDK2-Cyclin E regulating G1/S transition, CDK2-Cyclin A regulating S phase and G2 phase, CDK3-Cyclin C regulating G0/G1 phase, CDK4-Cyclin D regulating G1 phase, CDK5-p35 regulating transcription, CDK6-Cyclin D regulating G1 phase, CDK7-Cyclin H regulating transcription, CDK8-Cyclin C regulating transcription, CDK9-Cyclin T regulating transcription, and CDK11-Cyclin L, among others. CDKs phosphorylate their substrates on serine and threonine. A study has shown that the amino acid sequence of a CDK substrate is [S/T*]PX[K/R], wherein S/T* is the phosphorylated serine or threonine, P is proline, X is any amino acid, K is lysine, and R is arginine. A cyclin-dependent kinase inhibitor (CKI) interacts with a CDK-cyclin complex to block kinase activity, usually during G1 or in response to signals from the environment or from damaged DNA. In animal cells, there are two major CKI families, the INK4 family and the CIP/KIP family.

Taking oncogene and caner as a representative example, it is well-known that an oncogene is a gene that has the potential to cause cancer. In tumor cells, the consequences of dysregulation of various oncogenic and anti-oncogenic genes are expression or mutation of these genes at high level, and abnormal cell cycles. CDKs participate in different stages of cell cycles as a catalytic subunit and an important signal transporter within live cells. Previous studies indicated that aberrations during any stage of cell cycles will induce cell cycle dysfunction, which eventually will lead to the genesis of cancers. Therefore, the regulation and termination of tumor cell cycles is one of the most important strategies to treat cancers. Tumor development is closely associated with genetic alteration and deregulation of CDKs and their regulators, suggesting that inhibitors of CDKs may be useful anti-cancer therapeutics. CDK targeted anticancer therapies are superior over many current antitumor agents as they do not directly interact with DNA and have a low risk of secondary tumor development.

Among all CDK subtypes that participate in cell cycle regulation, CDKs 4 and 6 play a critical role. Cancer-related cell cycle aberrations mainly occur in G1 and G1 to S phase transition. Once CDK4/6-cyclin D complex is formed, it induces the phosphorylation of anti-oncogenic pRb which deactivates the pRb protein and releases the transcription factor bound, E2F, to direct the transcription of genes required for S phase, passing cell cycle checkpoints, and the G1 to S transition. The cyclin D-CDK4/6-INK4-Rr pathway is universally disrupted to favor cell proliferation in a majority of human cancer cases (˜80%). The disruption of this pathway enhances the cancer cell proliferation and overall survival by enhancing G1 cycle progression. Thus, the intervention against this pathway has been widely established as a promising cancer treatment strategy and CDK4/6 have been regarded as novel targets for anti-cancer drugs. The key advantages of CDK4/6 as new anti-cancer targets are as follows. The majority of proliferative cells rely on CDK4/6 and/or CDK2. However, the specific inhibition of CDK4/6 doesn't produce the cytotoxicity as observed for most pan-CDK inhibition such as bone marrow inhibition and gut reactions. Preclinical research has also demonstrated that, if two hallmarks of cancer cells are regulated, i.e. cellular cyclin D level is upregulated or P16INK4a is deactivated, the cells' sensitivity to drugs is increased, and consequently, the specificity of drugs against cancer cells can be improved.

Currently, representative CDK4/6 inhibitors in the market include Palbociclib from Pfizer (Approved in 2015), Ricociclib from Novartis (Phase III), and Abemaciclib from Eli Lilly (Phase III).

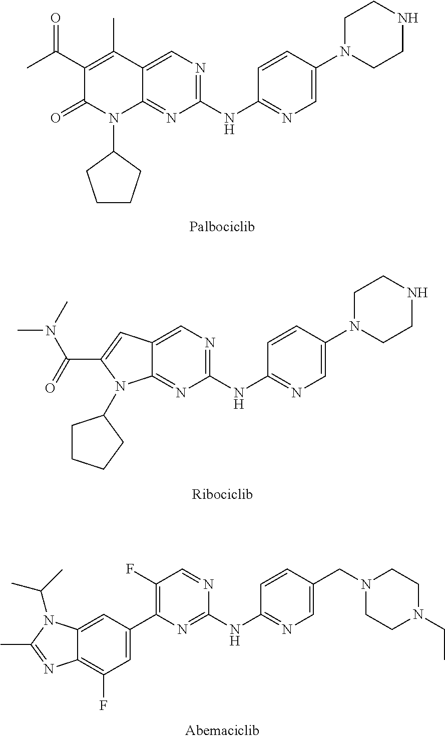 [SMILES: CC(=O)c1c(C)c2cnc(Nc3ccc(N4CCNCC4)cn3)nc2n(C2CCCC2)c1=O ; CN(C)C(=O)c1cc2cnc(Nc3ccc(N4CCNCC4)cn3)nc2n1C1CCCC1 ; CCN1CCN(Cc2ccc(Nc3ncc(F)c(-c4cc(F)c5nc(C)n(C(C)C)c5c4)n3)nc2)CC1]

CDK9 also participates in cell cycle regulation by regulating RNA transcription upon the formation of CDK9-cyclin T complex. As the catalytic subunit of a positive transcription elongation transcription factor, CDK9-cyclin T complex phosphorylates two elongation inhibitory factors of DSIF and NELF. There is no CDK9 inhibitor approved so far, except a few compounds on preclinical study stage. Despite their potent inhibitory activity against CDK9, most of these compounds exhibited inhibition against other CDKs, grave cytotoxicity, and strong side effects.

There continues to be a need for alternative CDK inhibitors for the treatment of various CDK-mediated diseases, particularly novel CDK inhibitors demonstrating lower toxicity, higher efficaciousness, and/or higher specificity, among others.

### SUMMARY OF THE INVENTION

One aspect of the present invention provides a fused bicyclic compound according to formula (H) or a pharmaceutically acceptable salt thereof:

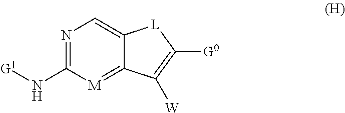

wherein L is —S— or —O—; M is —CH═, —N═ or —CJ=, wherein J is a non-hydrogen monovalent group; and W, G¹ and G¹ are independently of each other a non-hydrogen monovalent group.

In exemplary embodiments, the present invention provides a thieno[2,3-c]pyridine derivative with a Formula (H1), or a pharmaceutically acceptable salt thereof:

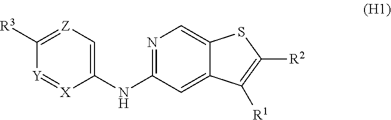

wherein:

X is CH or N;

R¹ is methyl, isopropyl, cyclopropyl, cyclobutyl, cyclopentyl, cyclohexyl, 1-methoxyl cyclopentyl, 1-cyclopentenyl, 3-bicyclo[3.1.0]hexyl, tetrahydrofuran-3-yl, or 3,3-difluorocyclopentyl;

R² is —CN,

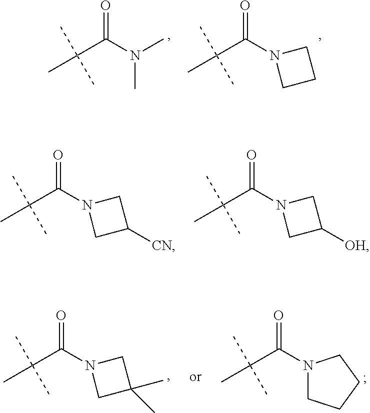

R³ is H,

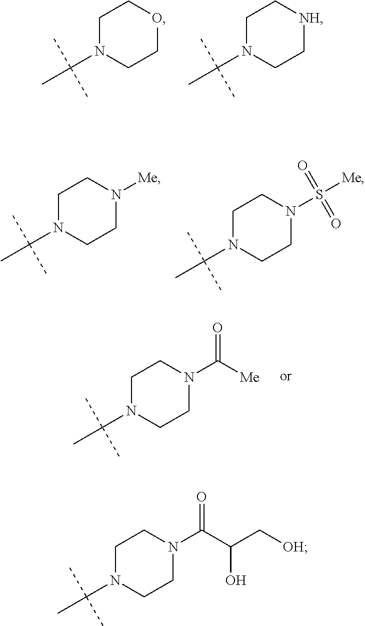

Y is N or CR⁴, wherein R⁴ is H,

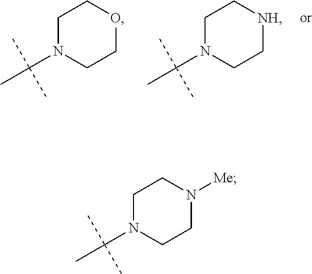

and

Z is CH or N.

Another aspect of the invention provides a pharmaceutical composition comprising a fused bicyclic compound of formula (H) or a pharmaceutically acceptable salt thereof, and optionally a pharmaceutically acceptable carrier, vehicle or diluent.

Still another aspect of the invention provides a method of synthesizing the compound of aforementioned formula (H1).

Still another aspect of the invention provides a method for treating a CDK-mediated disease or disorder in a mammal, which method comprises administering to said mammal a therapeutically effective amount of a fused bicyclic compound of formula (H) or a pharmaceutically acceptable salt thereof.

A further aspect of the invention provides a method for inhibiting CDK comprising contacting the CKD with a fused bicyclic compound of formula (H) or a pharmaceutically acceptable salt thereof.

Still another aspect of the invention provides a use of a fused bicyclic compound of formula (H) or a pharmaceutically acceptable salt thereof in the manufacturing of a medicament for treating a CDK-mediated disease or disorder.

The above features and advantages and other features and advantages of the present invention are readily apparent from the following detailed description of the best modes for carrying out the invention when taken in connection with the accompanying drawings.

### BRIEF DESCRIPTION OF THE SEVERAL VIEWS OF THE DRAWINGS

Not applicable.

### DETAILED DESCRIPTION OF THE PREFERRED EMBODIMENT

In the following description, for the purposes of explanation, numerous specific details are set forth in order to provide a thorough understanding of the present invention. It is apparent, however, to one skilled in the art that the present invention may be practiced without these specific details or with an equivalent arrangement.

At various places in the present specification, substituents of compounds of the invention are disclosed in groups or in ranges. It is specifically intended that the invention include each and every individual sub-combination of the members of such groups and ranges.

For example, the term “C₁₋₆ alkyl” is specifically intended to include C₁ alkyl (methyl), C₂ alkyl (ethyl), C₃ alkyl, C₄ alkyl, C₅ alkyl, and C₆ alkyl. Where a numerical range is disclosed herein, unless otherwise specified, such range is continuous, inclusive of both the minimum and maximum values of the range as well as every value between such minimum and maximum values. Still further, where a range refers to integers, only the integers from the minimum value to and including the maximum value of such range are included. In addition, where multiple ranges are provided to describe a feature or characteristic, such ranges can be combined.

The present invention provides the following embodiments.

### Embodiment #1

A fused bicyclic compound according to formula (H) or a pharmaceutically acceptable salt thereof:

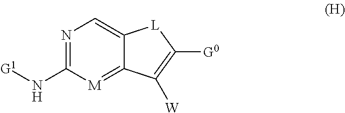

wherein L is —S— or —O—; M is —CH═, —N═ or —CJ=, wherein J is a non-hydrogen monovalent group; and W, G¹ and G¹ are independently of each other a non-hydrogen monovalent group.

The term “non-hydrogen monovalent group”, as used herein, may include, but is not limited to, one of the following groups. (1) Halo or halogen group, i.e. fluorine, chlorine, bromine or iodine. (2) Alkyl group, i.e. saturated aliphatic hydrocarbon including straight chains and branched chains. In some embodiments, the alkyl group has 1 to 20 carbon atoms, 1 to 10 carbon atoms, 1 to 6 carbon atoms, or 1 to 4 carbon atoms. For example, the term “C₁₋₆ alkyl” refers to linear or branched radicals of 1 to 6 carbon atoms (e.g., methyl, ethyl, n-propyl, isopropyl, n-butyl, isobutyl, sec-butyl, tert-butyl, n-pentyl, or n-hexyl). An alkyl group optionally can be substituted by one or more (e.g., 1 to 5) suitable substituents. (3) Haloalkyl group such as fluoroalkyl, i.e. an alkyl group having one or more halogen substituents such as F (up to perhaloalkyl, i.e., every hydrogen atom of the alkyl group has been replaced by a halogen atom). For example, C₁₋₆ haloalkyl is a C₁₋₆ alkyl group having one or more halogen substituents (up to perhaloalkyl, i.e., every hydrogen atom of the alkyl group has been replaced by a halogen atom). C₁ haloalkyl is a methyl group having one, two, or three halogen substituents. (4) Hydroxylalkyl or hydroxyalkyl, i.e. an alkyl group having one or more (e.g., 1, 2, or 3) OH substituents. (5) Cyanoalkyl group, i.e an alkyl group having one or more (e.g., 1, 2, or 3) —CN substituents. (6) Alkoxy or alkyloxy group, i.e. an —O-alkyl group. For example, the term C₁₋₆ alkoxy/alkyloxy is an —O—(C₁₋₆ alkyl) group. Examples of alkoxy include methoxy, ethoxy, propoxy (e.g., n-propoxy and isopropoxy), tert-butoxy, and the like. The alkoxy or alkyloxy group optionally can be substituted by 1 or more (e.g., 1 to 5) suitable substituents. (7) Haloalkoxy group such as fluoroalkoxy, i.e. an —O-haloalkyl group. For example, C₁₋₆ haloalkoxy refers to an —O—(C₁₋₆ haloalkyl) group. (8) Alkenyl group, i.e. aliphatic hydrocarbon having at least one carbon-carbon double bond, including straight chains and branched chains having at least one carbon-carbon double bond. In some embodiments, the alkenyl group has 2 to 20 carbon atoms, 2 to 10 carbon atoms, 2 to 6 carbon atoms, 3 to 6 carbon atoms, or 2 to 4 carbon atoms. For example, the term “C₂₋₆ alkenyl” includes straight or branched chain unsaturated radicals (having at least one carbon-carbon double bond) of 2 to 6 carbon atoms, including, but not limited to, ethenyl, 1-propenyl, 2-propenyl (allyl), isopropenyl, 2-methyl-1-propenyl, 1-butenyl, 2-butenyl, and the like. An alkenyl group optionally can be substituted by one or more (e.g., 1 to 5) suitable substituents. The alkenyl group may exist as the pure E form, the pure Z form, or any mixture thereof. (9) Alkynyl group, i.e. aliphatic hydrocarbons having at least one carbon-carbon triple bond, including straight chains and branched chains having at least one carbon-carbon triple bond. In some embodiments, the alkynyl group has 2 to 20, 2 to 10, 2 to 6, or 3 to 6 carbon atoms. For example, “C₂₋₆ alkynyl” includes straight or branched hydrocarbon chain alkynyl radicals as defined above, having 2 to 6 carbon atoms. An alkynyl group optionally can be substituted by one or more (e.g., 1 to 5) suitable substituents. (10) Cycloalkyl group, i.e. saturated or unsaturated, non-aromatic, monocyclic or polycyclic (such as bicyclic) hydrocarbon rings (e.g., monocyclics such as cyclopropyl, cyclobutyl, cyclopentyl, cyclohexyl, cycloheptyl, cyclooctyl, cyclononyl, or bicyclics including spiro, fused, or bridged systems (such as bicyclo[1.1.1]pentanyl, bicyclo[2.2.1]heptanyl, bicyclo[3.2.1]octanyl or bicyclo[5.2.0]nonanyl, decahydronaphthalenyl, etc.). The cycloalkyl group has 3 to 15 carbon atoms. In some embodiments the cycloalkyl may optionally contain one, two or more non-cumulative non-aromatic double or triple bonds and/or one to three oxo groups. In some embodiments, the bicycloalkyl group has 6 to 14 carbon atoms. For example, “C₃₋₁₄ cycloalkyl” includes saturated or unsaturated, non-aromatic, monocyclic or polycyclic (such as bicyclic) hydrocarbon rings of 3 to 14 ring-forming carbon atoms (e.g., cyclopropyl, cyclobutyl, cyclopentyl, cyclohexyl, bicyclo[1.1.1]pentanyl, or cyclodecanyl). The cycloalkyl group optionally can be substituted by 1 or more (e.g., 1 to 5) suitable substituents. (11) Aryl group, i.e. all-carbon monocyclic or fused-ring polycyclic aromatic groups having a conjugated pi-electron system. The aryl group may have 6 or 10 carbon atoms in the ring(s). Most commonly, the aryl group has 6 carbon atoms in the ring. For example, C₆₋₁₀ aryl is an aromatic radical containing from 6 to 10 carbon atoms such as phenyl or naphthyl. The aryl group optionally can be substituted by 1 or more (e.g., 1 to 5) suitable substituents. (12) Heteroaryl group, i.e. monocyclic or fused-ring polycyclic aromatic heterocyclic groups with one or more heteroatom ring members (ring-forming atoms) each independently selected from O, S and N in at least one ring. The heteroaryl group has 5 to 14 ring-forming atoms, including 1 to 13 carbon atoms, and 1 to 8 heteroatoms selected from O, S, and N. In some embodiments, the heteroaryl group has 5 to 10 ring-forming atoms including one to four heteroatoms. The heteroaryl group can also contain one to three oxo or thiono (i.e., ═S) groups. In some embodiments, the heteroaryl group has 5 to 8 ring-forming atoms including one, two or three heteroatoms. For example, 5-membered heteroaryl group is a monocyclic heteroaryl group as defined above with 5 ring-forming atoms in the monocyclic heteroaryl ring; 6-membered heteroaryl is a monocyclic heteroaryl group as defined above with 6 ring-forming atoms in the monocyclic heteroaryl ring; 5˜10-membered heteroaryl is a monocyclic or bicyclic heteroaryl group as defined above with 5, 6, 7, 8, 9 or 10 ring-forming atoms in the monocyclic or bicyclic heteroaryl ring. A heteroaryl group optionally can be substituted by 1 or more (e.g., 1 to 5) suitable substituents. Examples of monocyclic heteroaryls include those with 5 ring-forming atoms including one to three heteroatoms or those with 6 ring-forming atoms including one, two or three nitrogen heteroatoms. Examples of fused bicyclic heteroaryls include two fused 5- and/or 6-membered monocyclic rings including one to four heteroatoms. Examples of heteroaryl groups include pyridinyl, pyrazinyl, pyrimidinyl, pyridazinyl, thienyl, furyl, imidazolyl, pyrrolyl, oxazolyl (e.g., 1,3-oxazolyl, 1,2-oxazolyl), thiazolyl (e.g., 1,2-thiazolyl, 1,3-thiazolyl), pyrazolyl (e.g., pyrazol-1-yl, pyrazol-3-yl, pyrazol-4-yl), tetrazolyl, triazolyl (e.g., 1,2,3-triazolyl, 1,2,4-triazolyl), oxadiazolyl (e.g., 1,2,3-oxadiazolyl), thiadiazolyl (e.g., 1,3,4-thiadiazolyl), quinolyl, isoquinolyl, benzothienyl, benzofuryl, indolyl, 1H-imidazo[4,5-c]pyridinyl, imidazo[1,2-a]pyridinyl, 1H-pyrrolo[3,2-c]pyridinyl, imidazo[1,2-a]pyrazinyl, imidazo[2,1-c][1,2,4]triazinyl, imidazo[1,5-a]pyrazinyl, imidazo[1,2-a]pyrimidinyl, 1H-indazolyl, 9H-purinyl, imidazo[1,2-a]pyrimidinyl, [1,2,4]triazolo[1,5-a]pyrimidinyl, [1,2,4]triazolo[4,3-b]pyridazinyl, isoxazolo[5,4-c]pyridazinyl, isoxazolo[3,4-c]pyridazinyl, pyridone, pyrimidone, pyrazinone, pyrimidinone, 1H-imidazol-2(3H)-one, 1H-pyrrole-2,5-dione, 3-oxo-2H-pyridazinyl, 1H-2-oxo-pyrimidinyl, 1H-2-oxo-pyridinyl, 2,4(1H,3H)-dioxo-pyrimidinyl, 1H-2-oxo-pyrazinyl, and the like. (13) Heterocycloalkyl group, i.e. monocyclic or polycyclic (including 2 or more rings that are fused together, including spiro, fused, or bridged systems, for example, a bicyclic ring system), saturated or unsaturated, non-aromatic 4- to 15-membered ring system including 1 to 14 ring-forming carbon atoms and 1 to 10 ring-forming heteroatoms each independently selected from O, S, N, P and B. The heterocycloalkyl group can also optionally contain one or more oxo (i.e., ═O) or thiono (i.e., ═S) groups. For example, 4- to 12-membered heterocycloalkyl is a monocyclic or polycyclic, saturated or unsaturated, non-aromatic 4- to 12-membered ring system that comprises one or more ring-forming heteroatoms. Examples of such heterocycloalkyl rings include azetidinyl, tetrahydrofuranyl, imidazolidinyl, pyrrolidinyl, piperidinyl, piperazinyl, oxazolidinyl, thiazolidinyl, pyrazolidinyl, thiomorpholinyl, tetrahydrothiazinyl, tetrahydrothiadiazinyl, morpholinyl, oxetanyl, tetrahydrodiazinyl, oxazinyl, oxathiazinyl, quinuclidinyl, chromanyl, isochromanyl, benzoxazinyl, 2-oxaspiro[3.3]heptyl {e.g., 2-oxaspiro[3.3]hept-6-yl}, 7-azabicyclo[2.2.1]heptan-1-yl, 7-azabicyclo[2.2.1]heptan-2-yl, 7-azabicyclo[2.2.1]heptan-7-yl, 2-azabicyclo[2.2.1]heptan-3-on-2-yl, 3-azabicyclo[3.1.0]hexanyl, 3-azabicyclo[4.1.0]heptanyl and the like. Further examples of heterocycloalkyl rings include tetrahydrofuran-2-yl, tetrahydrofuran-3-yl, tetrahydropyranyl (e.g., tetrahydro-2H-pyran-4-yl), imidazolidin-1-yl, imidazolidin-2-yl, imidazolidin-4-yl, pyrrolidin-1-yl, pyrrolidin-2-yl, pyrrolidin-3-yl, piperidin-1-yl, piperidin-2-yl, piperidin-3-yl, piperidin-4-yl, piperazin-1-yl, piperazin-2-yl, 1,3-oxazolidin-3-yl, 1,4-oxazepan-1-yl, isothiazolidinyl, 1,3-thiazolidin-3-yl, 1,2-pyrazolidin-2-yl, 1,2-tetrahydrothiazin-2-yl, 1,3-thiazinan-3-yl, 1,2-tetrahydrodiazin-2-yl, 1,3-tetrahydrodiazin-1-yl, 1,4-oxazin-4-yl, oxazolidinonyl, 2-oxo-piperidinyl (e.g., 2-oxo-piperidin-1-yl), 2-oxoazepan-3-yl, and the like. Some examples of aromatic-fused heterocycloalkyl groups include indolinyl, isoindolinyl, isoindolin-1-one-3-yl, 5,7-dihydro-6H-pyrrolo[3,4-b]pyridin-6-yl, 6,7-dihydro-5H-pyrrolo[3,4-d]pyrimidin-6-yl, 4,5,6,7-tetrahydrothieno[2,3-c]pyridine-5-yl, 5,6-dihydrothieno[2,3-c]pyridin-7(4H)-one-5-yl, 1,4,5,6-tetrahydropyrrolo[3,4-c]pyrazol-5-yl, and 3,4-dihydroisoquinolin-(2H)-one-3-yl groups.

The heterocycloalkyl group is optionally substituted by 1 or more (e.g., 1 to 5) suitable substituents. Examples of heterocycloalkyl groups include 5- or 6-membered monocyclic rings and 9- or 10-membered fused bicyclic rings.

The point of attachment of the non-hydrogen monovalent group can be from any suitable position. For example, piperidinyl can be piperidin-1-yl (attached through the N atom of the piperidinyl), piperidin-2-yl (attached through the C atom at the 2-position of the piperidinyl), piperidin-3-yl (attached through the C atom at the 3-position of the piperidinyl), or piperidin-4-yl (attached through the C atom at the 4-position of the piperidinyl). For another example, pyridinyl (or pyridyl) can be 2-pyridinyl (or pyridin-2-yl), 3-pyridinyl (or pyridin-3-yl), or 4-pyridinyl (or pyridin-4-yl). The point of attachment of the non-hydrogen monovalent group can be specified to indicate the position where the non-hydrogen monovalent group is attached to another moiety. For example, “—C₁₋₂ alkyl-(C₃₋₄ cycloalkyl)” means the point of attachment occurs at the “C₁₋₂ alkyl” part. For another example, “(C₃₋₄ cycloalkyl)-C₁₋₂ alkyl-” also means the point of attachment occurs at the “C₁₋₂ alkyl” part. When a bond to a substituent is shown to cross a bond connecting two atoms in a ring, then such substituent may be bonded to any of the ring-forming atoms in that ring that are substitutable (i.e., one or more hydrogen atoms), unless otherwise specified or otherwise implicit from the context.

The present invention includes all pharmaceutically acceptable isotopically labelled compounds of Formula H or salts thereof, wherein one or more atoms are replaced by atoms having the same atomic number, but an atomic mass or mass number different from the atomic mass or mass number which predominates in nature. Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically labelled compounds of Formula H, for example, those incorporating a radioactive isotope, are useful in drug and/or substrate tissue distribution studies. The radioactive isotopes tritium, i.e., ³H, and carbon-14, i.e., 14C, are particularly useful for this purpose in view of their ease of incorporation and detection. Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances. Substitution with positron-emitting isotopes, such as ¹¹C, ¹⁸F, ¹⁵O and ¹³N, can be useful in Positron Emission Topography (PET) studies for examining substrate receptor occupancy. Isotopically labeled compounds of Formula H can generally be prepared by conventional techniques known to those skilled in the art using an appropriate isotopically labeled reagent in place of the non-labeled reagent previously employed.

Some compounds of Formula H may include stereoisomers and tautomers, all of which are included within the scope of the invention. Stereoisomers of Formula H include cis and trans isomers, optical isomers such as R and S enantiomers, diastereomers, geometric isomers, rotational isomers, atropisomers, and conformational isomers of the compounds of Formula H, including compounds exhibiting more than one type of isomerism; and mixtures thereof (such as racemates and diastereomeric pairs).

The compounds of Formula H may exist in the form of pharmaceutically acceptable salts such as acid addition salts and/or base salts thereof. Suitable acid addition salts are formed from acids which form non-toxic salts. Examples include hydrochloride/chloride, acetate, adipate, aspartate, benzoate, besylate, bicarbonate/carbonate, bisulfate/sulfate, borate, camphorsulfonate, citrate, cyclamate, edisylate, esylate, formate, fumarate, gluceptate, gluconate, glucuronate, hexafluorophosphate, hibenzate, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/di hydrogen phosphate, pyroglutamate, saccharate, stearate, succinate, tannate, tartrate, tosylate, trifluoroacetate and xinafoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts.

The compounds of Formula H or a pharmaceutically acceptable salt thereof include all forms of the compound of Formula H or pharmaceutically salt thereof, including hydrates, solvates, isomers (e.g. rotational stereoisomers), crystalline and non-crystalline forms, isomorphs, polymorphs, metabolites, and prodrugs thereof. Compounds of Formula H may exist in unsolvated and solvated forms. When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions.

Compounds of Formula H may exist in a continuum of solid states ranging from fully amorphous to fully crystalline. The term “amorphous” refers to a state in which the material lacks long-range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from apparent solid to a material with liquid properties occurs, which is characterized by a change of state, typically second order (“glass transition”). The term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (“melting point”). The compounds of the invention may also exist in a mesomorphic state (mesophase or liquid crystal) when subjected to suitable conditions. The mesomorphic state is intermediate between the true crystalline state and the true liquid state (either melt or solution).

### Embodiment #2

The fused bicyclic compound according to Embodiment #1 or a pharmaceutically acceptable salt thereof, wherein L is —S—.

### Embodiment #3

The fused bicyclic compound according to any one of Embodiments from #1 to #2 or a pharmaceutically acceptable salt thereof, wherein M is —CH═.

### Embodiment #4

The fused bicyclic compound according to any one of Embodiments from #1 to #3 or a pharmaceutically acceptable salt thereof, wherein W is C₁₋₆ alkyl such as methyl, ethyl, propyl, and isopropyl; monocyclic C₃₋₁₄ cycloalkyl such as cyclopropyl, cyclobutyl, cyclopentyl, and cyclohexyl; C₁₋₆ alkoxy-C₃₋₁₄ cycloalkyl such as 1-methoxyl cyclopentyl; C₂₋₆ alkynyl such as 1-cyclopentenyl; bicyclic C₆₋₁₄ cycloalkyl such as 3-bicyclo[3.1.0]hexyl; heterocycloalkyl such as tetrahydrofuran-3-yl; or monocyclic C₃₋₁₄ halocycloalkyl such as 3,3-difluorocyclopentyl.

### Embodiment #5

The fused bicyclic compound according to any one of Embodiments from #1 to #4 or a pharmaceutically acceptable salt thereof, wherein G¹ has a formula (G^{1a}):

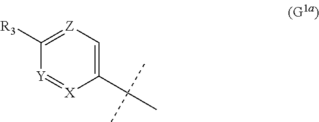

wherein R³ is H or a non-hydrogen monovalent group as defined in Embodiment #1, for example, heterocycloalkyl such as morpholino such as 4-morpholino

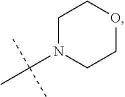

piperazinyl such as piperazin-1-yl

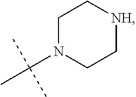

C₁₋₆-alkylpiperazinyl such as 4-methyl-piperazin-1-yl

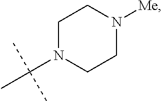

sulfonylpiperazinyl such as 4-(ethylsulfonyl)piperazin-1-yl, 4-(methylsulfonyl)piperazin-1-yl

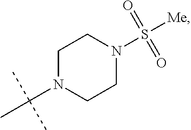

acylpiperazinyl such as acetylpiperazinyl such as 4-acetylpiperazin-1-yl

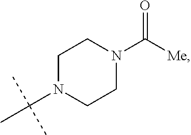

or hydroxylacylpiperazinyl such as 4-(2,3-dihydroxypropanoyl)-piperazin-1-yl

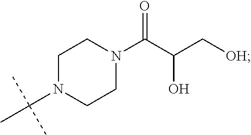

X is CH or N;
Y is N or CR⁴, wherein R⁴ is H or a non-hydrogen monovalent group as defined in Embodiment #1, for example, heterocycloalkyl such as morpholino such as 4-morpholino

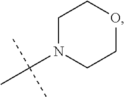

piperazinyl such as piperazin-1-yl

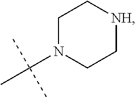

or C₁₋₆alkylpiperazinyl such as 4-methyl-piperazin-1-yl

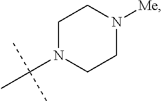

and

Z is CH or N.

### Embodiment #6

The fused bicyclic compound according to any one of Embodiments from #1 to #5 or a pharmaceutically acceptable salt thereof, wherein G¹ has a formula (G^{1b}):

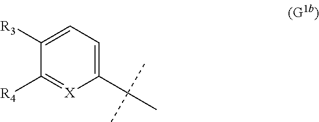

wherein X is CH or N;

R³ is H or a non-hydrogen monovalent group as defined in Embodiment #1, for example, heterocycloalkyl such as morpholino such as 4-morpholino

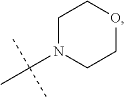

piperazinyl such as piperazin-1-yl

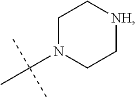

C₁₋₆alkylpiperazinyl such as 4-ethyl-piperazin-1-yl, 4-methyl-piperazin-1-yl

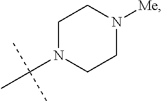

sulfonylpiperazinyl such as 4-(ethylsulfonyl)piperazin-1-yl, 4-(methylsulfonyl)piperazin-1-yl

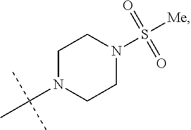

or
acylpiperazinyl such as acetylpiperazinyl such as 4-acetylpiperazin-1-yl

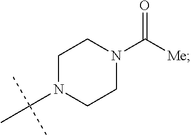

and

R⁴ is H or a non-hydrogen monovalent group as defined in Embodiment #1, for example, heterocycloalkyl such as morpholino such as 4-morpholino

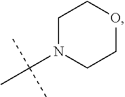

piperazinyl such as piperazin-1-yl

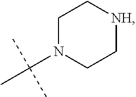

or C₁₋₆alkylpiperazinyl such as 4-methyl-piperazin-1-yl

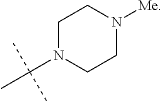

### Embodiment #7

The fused bicyclic compound according to any one of Embodiments from #1 to #6 or a pharmaceutically acceptable salt thereof, wherein G⁰ is cyano —CN; N,N-dialkylaminocarbonyl such as

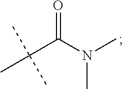

azetidinylcarbonyl such as

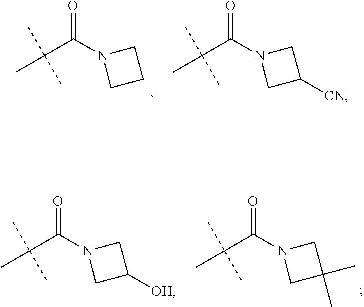

or pyrrolidinylcarbonyl such as 1-pyrrolidinylcarbonyl

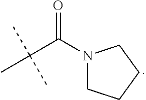

### Embodiment #8

The fused bicyclic compound according to Embodiment #1, which is a thieno[2,3-c]pyridine derivative with a Formula (H1), or a pharmaceutically acceptable salt thereof:

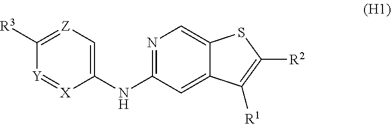

wherein:

X is CH or N;

R¹ is methyl, isopropyl, cyclopropyl, cyclobutyl, cyclopentyl, cyclohexyl, 1-methoxyl cyclopentyl, 1-cyclopentenyl, 3-bicyclo[3.1.0]hexyl, tetrahydrofuran-3-yl, or 3,3-difluorocyclopentyl;

R² is —CN,

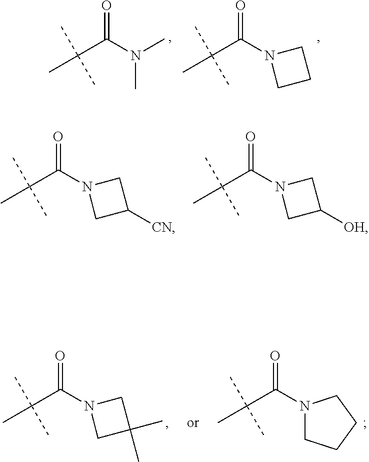

R³ is H,

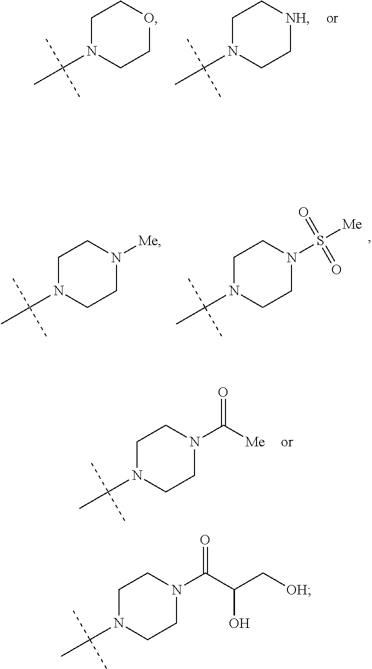

Y is N or CR⁴, wherein R⁴ is H,

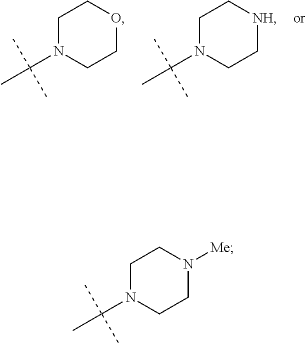

and

Z is CH or N.

### Embodiment #9

The fused bicyclic compound according to Embodiment #1 having a Formula (I), or a pharmaceutically acceptable salt thereof:

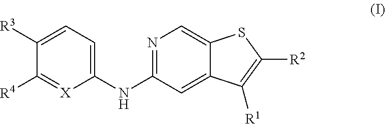

wherein:

X is CH or N;

R¹ is methyl, isopropyl, cyclopropyl, cyclobutyl, cyclopentyl, cyclohexyl, 1-methoxyl cyclopentyl, 1-cyclopentenyl, or 3-bicyclo[3.1.0]hexyl, tetrahydrofuran-3-yl, or 3,3-difluorocyclopentyl;

R² is

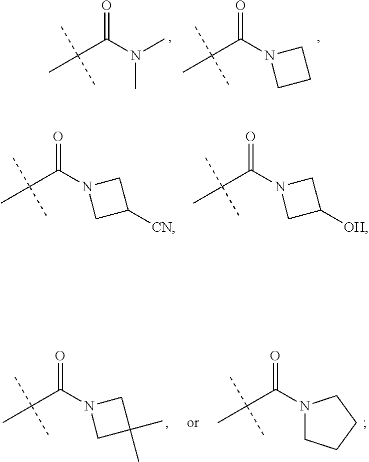

R³ is H,

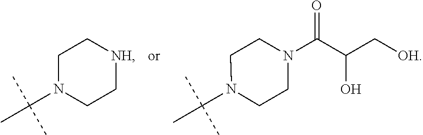

and

R⁴ is H,

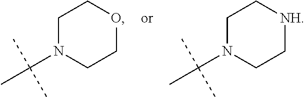

### Embodiment #10

The fused bicyclic compound according to Embodiment #9 or a pharmaceutically acceptable salt thereof, wherein X is N, R⁴ is H, and R³ is

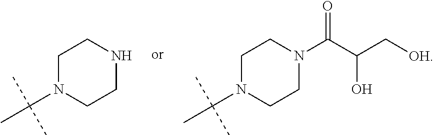

### Embodiment #11

The fused bicyclic compound according to any one of Embodiments from #9 to #10 or a pharmaceutically acceptable salt thereof, wherein X is CH, R³ is H, and R⁴ is

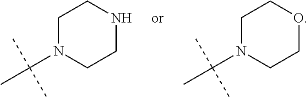

### Embodiment #12

The fused bicyclic compound according to Embodiment #1 or a pharmaceutically acceptable salt thereof, wherein the compound is selected from the group consisting of the following compounds:

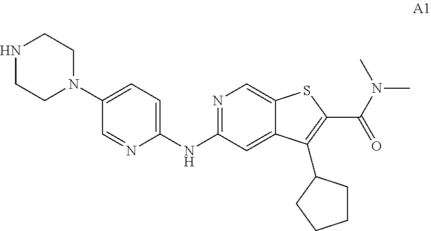 [SMILES: CN(C)C(=O)c1sc2cnc(Nc3ccc(N4CCNCC4)cn3)cc2c1C1CCCC1]

3-cyclopentyl-N,N-dimethyl-5-((5-(piperazin-1-yl)pyridin-2-yl)amino)thieno[2,3-c]pyridine-2-carboxamide (A1),
3-cyclopentyl-N,N-diethyl-5-((5-(piperazin-1-yl)pyridin-2-yl)amino)thieno[2,3-c]pyridine-2-carboxamide (A1a),
3-cyclopentyl-N-methyl-N-ethyl-5-((5-(piperazin-1-yl)pyridin-2-yl)amino)thieno[2,3-c]pyridine-2-carboxamide (A1b),

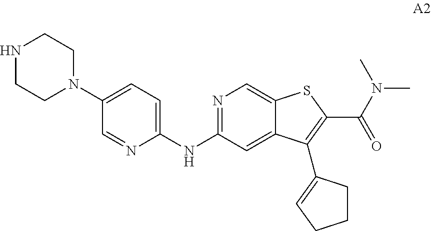 [SMILES: CN(C)C(=O)c1sc2cnc(Nc3ccc(N4CCNCC4)cn3)cc2c1C1=CCCC1]

3-(cyclopent-1-en-1-yl)-N,N-dimethyl-5-((5-(piperazin-1-yl)pyridin-2-yl)amino)thieno[2,3-c]pyridine-2-carboxamide (A2),
3-(cyclopent-1-en-3-yl)-N,N-dimethyl-5-((5-(piperazin-1-yl)pyridin-2-yl)amino)thieno[2,3-c]pyridine-2-carboxamide (A2a),
3-(cyclopent-1-en-4-yl)-N,N-dimethyl-5-((5-(piperazin-1-yl)pyridin-2-yl)amino)thieno[2,3-c]pyridine-2-carboxamide (A2b),
3-(cyclopent-1-en-1-yl)-N,N-diethyl-5-((5-(piperazin-1-yl)pyridin-2-yl)amino)thieno[2,3-c]pyridine-2-carboxamide (A2c),
3-(cyclopent-1-en-1-yl)-N-methyl-N-ethyl-5-((5-(piperazin-1-yl)pyridin-2-yl)amino)thieno[2,3-c]pyridine-2-carboxamide (A2d),

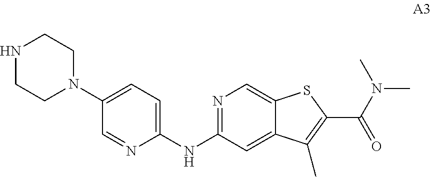 [SMILES: Cc1c(C(=O)N(C)C)sc2cnc(Nc3ccc(N4CCNCC4)cn3)cc12]

N,N,3-trimethyl-5-((5-(piperazin-1-yl)pyridin-2-yl)amino)thieno[2,3-c]pyridine-2-carboxamide (A3),
N,N-dimethyl-3-ethyl-5-((5-(piperazin-1-yl)pyridin-2-yl)amino)thieno[2,3-c]pyidine-2-carboxamide (A3a),
N,N-dimethyl-3-propyl-5-((5-(piperazin-1-yl)pyridin-2-yl)amino)thieno[2,3-c]pyridine-2-carboxamide (A3b),
N,N-dimethyl-3-isopropyl-5-((5-(piperazin-1-yl)pyridin-2-yl)amino)thieno[2,3-c]pyridine-2-carboxamide (A3c),
N,N,-diethyl-3-methyl-5-((5-(piperazin-1-yl)pyridin-2-yl)amino)thieno[2,3-c]pyridine-2-carboxamide (A3d),
N-ethyl-N,3-dimethyl-5-((5-(piperazin-1-yl)pyridin-2-yl)amino)thieno[2,3-c]pyridine-2-carboxamide (A3e),

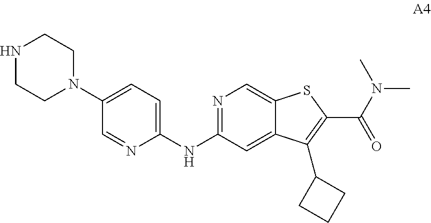 [SMILES: CN(C)C(=O)c1sc2cnc(Nc3ccc(N4CCNCC4)cn3)cc2c1C1CCC1]

3-cyclobutyl-N,N-dimethyl-5-((5-(piperazin-1-yl)pyridin-2-yl)amino)thieno[2,3-c]pyridine-2-carboxamide (A4),
3-cyclobutyl-N,N-diethyl-5-((5-(piperazin-1-yl)pyridin-2-yl)amino)thieno[2,3-c]pyridine-2-carboxamide (A4a),
3-cyclobutyl-N-methyl-N-ethyl-5-((5-(piperazin-1-yl)pyridin-2-yl)amino)thieno[2,3-c]pyridine-2-carboxamide (A4b),

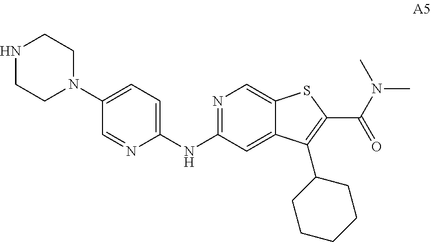 [SMILES: CN(C)C(=O)c1sc2cnc(Nc3ccc(N4CCNCC4)cn3)cc2c1C1CCCCC1]

3-cyclohexyl-N,N-dimethyl-5-((5-(piperazin-1-yl)pyridin-2-yl)amino)thieno[2,3-c]pyridine-2-carboxamide (A5),
3-cyclohexyl-N,N-diethyl-5-((5-(piperazin-1-yl)pyridin-2-yl)amino)thieno[2,3-c]pyridine-2-carboxamide (A5a),
3-cyclohexyl-N-methyl-N-ethyl-5-((5-(piperazin-1-yl)pyridin-2-yl)amino)thieno[2,3-c]pyridine-2-carboxamide (A5b),

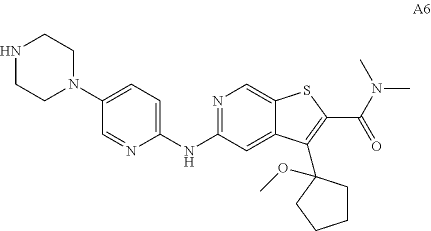 [SMILES: COC1(c2c(C(=O)N(C)C)sc3cnc(Nc4ccc(N5CCNCC5)cn4)cc23)CCCC1]

3-(1-methoxycyclopentyl)-N,N-dimethyl-5-((5-(piperazin-1-yl)pyridin-2-yl)amino)thieno[2,3-c]pyridine-2-carboxamide (A6),
3-(1-ethoxycyclopentyl)-N,N-dimethyl-5-((5-(piperazin-1-yl)pyridin-2-yl)amino)thieno[2, 3-c]pyridine-2-carboxamide (A6a),
3-(2-methoxycyclopentyl)-N,N-dimethyl-5-((5-(piperazin-1-yl)pyridin-2-yl)amino)thieno[2,3-c]pyridine-2-carboxamide (A6b),
3-(3-methoxycyclopentyl)-N,N-dimethyl-5-((5-(piperazin-1-yl)pyridin-2-yl)amino)thieno[2,3-c]pyridine-2-carboxamide (A6c),
3-(1-methoxycyclopentyl)-N,N-diethyl-5-((5-(piperazin-1-yl)pyridin-2-yl)amino)thieno[2,3-c]pyridine-2-carboxamide (A6d),
3-(1-methoxycyclopentyl)-N-methyl-N-ethyl-5-((5-(piperazin-1-yl)pyridin-2-yl)amino)thieno[2,3-c]pyridine-2-carboxamide (A6e),

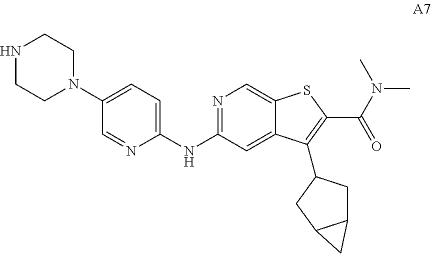 [SMILES: CN(C)C(=O)c1sc2cnc(Nc3ccc(N4CCNCC4)cn3)cc2c1C1CC2CC2C1]

3-(bicyclo[3.1.0]hexan-3-yl)-N,N-dimethyl-5-((5-(piperazin-1-yl)pyridin-2-yl)amino)thieno[2,3-c]pyridine-2-carboxamide (A7),
3-(bicyclo[3.1.0]hexan-3-yl)-N,N-diethyl-5-((5-(piperazin-1-yl)pyridin-2-yl)amino)thieno[2,3-c]pyridine-2-carboxamide (A7a),
3-(bicyclo[3.1.0]hexan-3-yl)-N-methyl-N-ethyl-5-((5-(piperazin-1-yl)pyridin-2-yl)amino)thieno[2,3-c]pyridine-2-carboxamide (A7b),

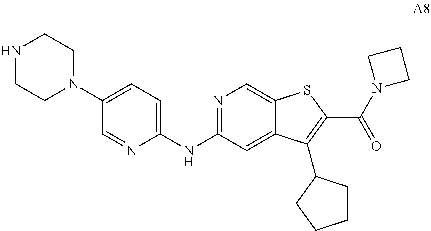 [SMILES: O=C(c1sc2cnc(Nc3ccc(N4CCNCC4)cn3)cc2c1C1CCCC1)N1CCC1]

azetidin-1-yl(3-cyclopentyl-5-((5-(piperazin-1-yl)pyridin-2-yl)amino)thieno[2,3-c]pyridin-2-yl)methanone (A8),
azetidin-2-yl(3-cyclopentyl-5-((5-(piperazin-1-yl)pyridin-2-yl)amino)thieno[2, 3-c]pyridin-2-yl)methanone (A8a),
azetidin-3-yl(3-cyclopentyl-5-((5-(piperazin-1-yl)pyridin-2-yl)amino)thieno[2,3-c]pyridin-2-yl)methanone (A8b),
azetidin-1-yl(3-cyclobutyl-5-((5-(piperazin-1-yl)pyridin-2-yl)amino)thieno[2,3-c]pyridin-2-yl) methanone (A8c),
azetidin-1-yl(3-cyclohexyl-5-((5-(piperazin-1-yl)pyridin-2-yl)amino)thieno[2,3-c]pyridin-2-yl)methanone (A8d),

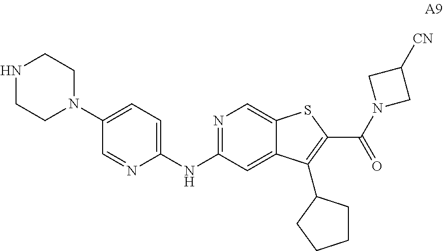 [SMILES: N#CC1CN(C(=O)c2sc3cnc(Nc4ccc(N5CCNCC5)cn4)cc3c2C2CCCC2)C1]

1-(3-cyclopentyl-5-((5-(piperazin-1-yl)pyridin-2-yl)amino)thieno[2,3-c]pyridine-2-carbonyl) azetidine-3-carbonitrile (A9),
1-(3-cyclobutyl-5-((5-(piperazin-1-yl)pyridin-2-yl)amino)thieno[2,3-c]pyridine-2-carbonyl)azetidine-3-carbonitrile (A9a),
1-(3-cyclohexyl-5-((5-(piperazin-1-yl)pyridin-2-yl)amino)thieno[2,3-c]pyridine-2-carbonyl) azetidine-3-carbonitrile (A9b),

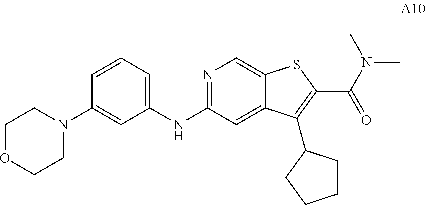 [SMILES: CN(C)C(=O)c1sc2cnc(Nc3cccc(N4CCOCC4)c3)cc2c1C1CCCC1]

3-cyclopentyl-N,N-dimethyl-5-((3-morpholinophenyl)amino)thieno[2,3-c]pyridine-2-carboxamide (A10),
3-cyclobutyl-N,N-dimethyl-5-((3-morpholinophenyl)amino)thieno[2,3-c]pyridine-2-carboxamide (A10a),
3-cyclohexyl-N,N-dimethyl-5-((3-morpholinophenyl)amino)thieno[2,3-c]pyridine-2-carboxamide (A10b),
3-cyclopentyl-N,N-diethyl-5-((3-morpholinophenyl)amino)thieno[2,3-c]pyridine-2-carboxamide (A10c),
3-cyclopentyl-N-methyl-N-ethyl-5-((3-morpholinophenyl)amino)thieno[2,3-c]pyridine-2-carboxamide (A10d),

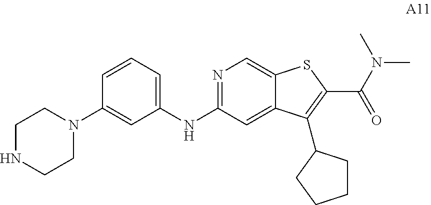 [SMILES: CN(C)C(=O)c1sc2cnc(Nc3cccc(N4CCNCC4)c3)cc2c1C1CCCC1]

3-cyclopentyl-N,N-dimethyl-5-((3-(piperazin-1-yl)phenyl)amino)thieno[2,3-c]pyridine-2-carboxamide (A11),
3-cyclobutyl-N,N-dimethyl-5-((3-(piperazin-1-yl)phenyl)amino)thieno[2,3-c]pyridine-2-carboxamide (A11a),
3-cyclohexyl-N,N-dimethyl-5-((3-(piperazin-1-yl)phenyl)amino)thieno[2, 3-c]pyridine-2-carboxamide (A11b),
3-cyclopentyl-N,N-diethyl-5-((3-(piperazin-1-yl)phenyl)amino)thieno[2,3-c]pyridine-2-carboxamide (A11c),
3-cyclopentyl-N-methyl-N-ethyl-5-((3-(piperazin-1-yl)phenyl)amino)thieno[2,3-c]pyridine-2-carboxamide (A11d),

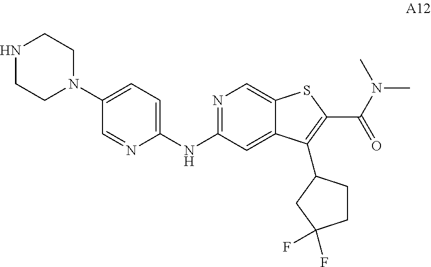 [SMILES: CN(C)C(=O)c1sc2cnc(Nc3ccc(N4CCNCC4)cn3)cc2c1C1CCC(F)(F)C1]

3-(3,3-difluorocyclopentyl)-N,N-dimethyl-5-((5-(piperazin-1-yl)pyridin-2-yl)amino)thieno[2,3-c]pyridine-2-carboxamide (A12),
3-(3,4-difluorocyclopentyl)-N,N-dimethyl-5-((5-(piperazin-1-yl)pyridin-2-yl)amino)thieno[2,3-c]pyridine-2-carboxamide (A12a),
3-(2,5-difluorocyclopentyl)-N,N-dimethyl-5-((5-(piperazin-1-yl)pyridin-2-yl)amino)thieno[2,3-c]pyridine-2-carboxamide (A12b),
3-(2,2-difluorocyclopentyl)-N,N-dimethyl-5-((5-(piperazin-1-yl)pyridin-2-yl)amino)thieno[2,3-c]pyridine-2-carboxamide (A12c),
3-(2,3-difluorocyclopentyl)-N,N-dimethyl-5-((5-(piperazin-1-yl)pyridin-2-yl)amino)thieno[2,3-c]pyridine-2-carboxamide (A12d),
3-(3,3-dichlorocyclopentyl)-N,N-dimethyl-5-((5-(piperazin-1-yl)pyridin-2-yl)amino)thieno[2,3-c]pyridine-2-carboxamide (A12e),
3-(3,3-difluorocyclopentyl)-N,N-diethyl-5-((5-(piperazin-1-yl)pyridin-2-yl)amino)thieno[2,3-c]pyridine-2-carboxamide (A12f),
3-(3,3-difluorocyclopentyl)-N-methyl-N-ethyl-5-((5-(piperazin-1-yl)pyridin-2-yl)amino)thieno[2,3-c]pyridine-2-carboxamide (A12g),
3-(2,2-difluorocyclobutyl)-N,N-dimethyl-5-((5-(piperazin-1-yl)pyridin-2-yl)amino)thieno[2,3-c]pyridine-2-carboxamide (A12h),
3-(2,2-difluorocyclohexyl)-N,N-dimethyl-5-((5-(piperazin-1-yl)pyridin-2-yl)amino)thieno[2,3-c]pyridine-2-carboxamide (A12i),
3-(3,3-difluorocyclohexyl)-N,N-dimethyl-5-((5-(piperazin-1-yl)pyridin-2-yl)amino)thieno[2,3-c]pyridine-2-carboxamide (A12j),

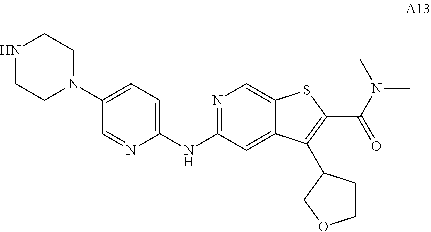 [SMILES: CN(C)C(=O)c1sc2cnc(Nc3ccc(N4CCNCC4)cn3)cc2c1C1CCOC1]

N,N-dimethyl-5-((5-(piperazin-1-yl)pyridin-2-yl)amino)-3-(tetrahydrofuran-3-yl)thieno[2,3-c]pyridine-2-carboxamide (A13),
N,N-dimethyl-5-((5-(piperazin-1-yl)pyridin-2-yl)amino)-3-(tetrahydrofuran-2-yl)thieno[2,3-c]pyridine-2-carboxamide (A13a),
N,N-diethyl-5-((5-(piperazin-1-yl)pyridin-2-yl)amino)-3-(tetrahydrofuran-3-yl)thieno[2,3-c]pyridine-2-carboxamide (A13b),
N-methyl-N-ethyl-5-((5-(piperazin-1-yl)pyridin-2-yl)amino)-3-(tetrahydrofuran-3-yl)thieno[2,3-c]pyridine-2-carboxamide (A13c),

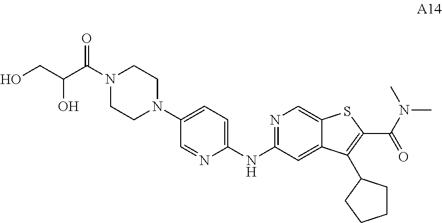 [SMILES: CN(C)C(=O)c1sc2cnc(Nc3ccc(N4CCN(C(=O)C(O)CO)CC4)cn3)cc2c1C1CCCC1]

3-cyclopentyl-5-((5-(4-(2,3-dihydroxypropanoyl)piperazin-1-yl)pyridin-2-yl)amino)-N,N-dimethylthieno[2,3-c]pyridine-2-carboxamide (A14),
3-cyclobutyl-5-((5-(4-(2,3-dihydroxypropanoyl)piperazin-1-yl)pyridin-2-yl)amino)-N,N-dimethylthieno[2,3-c]pyridine-2-carboxamide (A14a),
3-cyclohexyl-5-((5-(4-(2,3-dihydroxypropanoyl)piperazin-1-yl)pyridin-2-yl)amino)-N,N-dimethylthieno[2,3-c]pyridine-2-carboxamide (A14b),
3-cyclopentyl-5-((5-(4-(2,3-dihydroxypropanoyl)piperazin-1-yl)pyridin-2-yl)amino)-N,N-diethylthieno[2,3-c]pyridine-2-carboxamide (A14c),
3-cyclopentyl-5-((5-(4-(2,3-dihydroxypropanoyl)piperazin-1-yl)pyridin-2-yl)amino)-N-methyl-N-ethylthieno[2,3-c]pyridine-2-carboxamide (A14d),

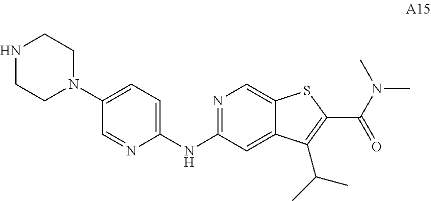 [SMILES: CC(C)c1c(C(=O)N(C)C)sc2cnc(Nc3ccc(N4CCNCC4)cn3)cc12]

3-isopropyl-N,N-dimethyl-5-((5-(piperazin-1-yl)pyridin-2-yl)amino)thieno[2,3-c]pyridine-2-carboxamide (A15),
3-propyl-N,N-dimethyl-5-((5-(piperazin-1-yl)pyridin-2-yl)amino)thieno[2,3-c]pyridine-2-carboxamide (A15a),
3-n-butyl-N,N-dimethyl-5-((5-(piperazin-1-yl)pyridin-2-yl)amino)thieno[2,3-c]pyridine-2-carboxamide (A15b),
3-isobutyl-N,N-dimethyl-5-((5-(piperazin-1-yl)pyridin-2-yl)amino)thieno[2,3-c]pyridine-2-carboxamide (A15c),
3-isopropyl-N,N-diethyl-5-((5-(piperazin-1-yl)pyridin-2-yl)amino)thieno[2,3-c]pyridine-2-carboxamide (A15d),
3-isopropyl-N-methyl-N-ethyl-5-((5-(piperazin-1l-yl)pyridin-2-yl)amino)thieno[2,3-c]pyridine-2-carboxamide (A15e),

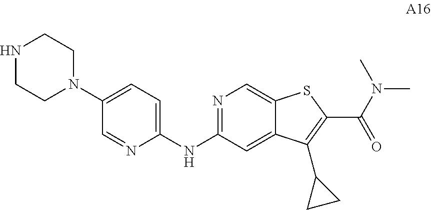 [SMILES: CN(C)C(=O)c1sc2cnc(Nc3ccc(N4CCNCC4)cn3)cc2c1C1CC1]

3-cyclopropyl-N,N-dimethyl-5-((5-(piperazin-1-yl)pyridin-2-yl)amino)thieno[2,3-c]pyridine-2-carboxamide (A16),
3-cyclopropyl-N,N-diethyl-5-((5-(piperazin-1-yl)pyridin-2-yl)amino)thieno[2,3-c]pyridine-2-carboxamide (A16a),
3-cyclopropyl-N-methyl-N-ethyl-5-((5-(piperazin-1-yl)pyridin-2-yl)amino)thieno[2,3-c]pyridine-2-carboxamide (A16b),

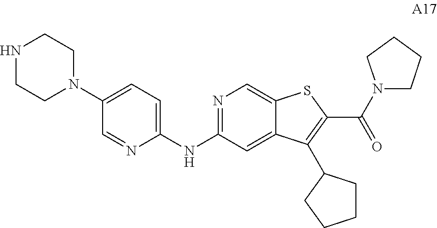 [SMILES: O=C(c1sc2cnc(Nc3ccc(N4CCNCC4)cn3)cc2c1C1CCCC1)N1CCCC1]

(3-cyclopentyl-5-((5-(piperazin-1-yl)pyridin-2-yl)amino)thieno[2,3-c]pyridin-2-yl)(pyrrolidin-1-yl)methanone (A17),
(3-cyclopentyl-5-((5-(piperazin-1-yl)pyridin-2-yl)amino)thieno[2,3-c]pyridin-2-yl)(pyrrolidin-2-yl)methanone (A17a),
(3-cyclopropyl-5-((5-(piperazin-1-yl)pyridin-2-yl)amino)thieno[2,3-c]pyridin-2-yl)(pyrrolidin-1-yl)methanone (A17b),
(3-cyclobutyl-5-((5-(piperazin-1-yl)pyridin-2-yl)amino)thieno[2,3-c]pyridin-2-yl)(pyrrolidin-1-yl)methanone (A17c),
(3-cyclohexyl-5-((5-(piperazin-1-yl)pyridin-2-yl)amino)thieno[2,3-c]pyridin-2-yl)(pyrrolidin-1-yl)methanone (A17d),

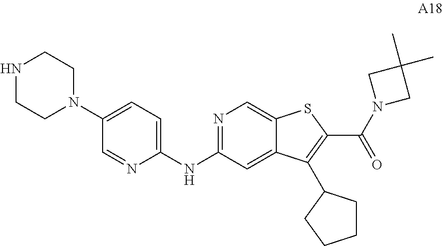 [SMILES: CC1(C)CN(C(=O)c2sc3cnc(Nc4ccc(N5CCNCC5)cn4)cc3c2C2CCCC2)C1]

(3-cyclopentyl-5-((5-(piperazin-1-yl)pyridin-2-yl)amino)thieno[2,3-c]pyridin-2-yl)(3,3-dimethylazetidin-1-yl)methanone (A18),
(3-cyclopropyl-5-((5-(piperazin-1-yl)pyridin-2-yl)amino)thieno[2,3-c]pyridin-2-yl)(3,3-dimethylazetidin-1-yl)methanone (A18a),
(3-cyclobutyl-5-((5-(piperazin-1-yl)pyridin-2-yl)amino)thieno[2,3-c]pyridin-2-yl)(3,3-dimethylazetidin-1-yl)methanone (A18b),
(3-cyclohexyl-5-((5-(piperazin-1-yl)pyridin-2-yl)amino)thieno[2,3-c]pyridin-2-yl)(3,3-dimethylazetidin-1-yl)methanone (A18c),
(3-cyclopentyl-5-((5-(piperazin-1-yl)pyridin-2-yl)amino)thieno[2,3-c]pyridin-2-yl)(3,3-diethylazetidin-1-yl)methanone (A18d),
(3-cyclopentyl-5-((5-(piperazin-1-yl)pyridin-2-yl)amino)thieno[2,3-c]pyridin-2-yl)(3-methyl-3-ethyl-azetidin-1-yl)methanone (A18e),

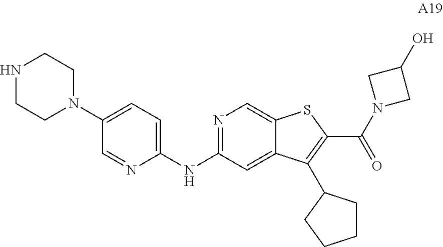 [SMILES: O=C(c1sc2cnc(Nc3ccc(N4CCNCC4)cn3)cc2c1C1CCCC1)N1CC(O)C1]

(3-cyclopentyl-5-((5-(piperazin-1-yl)pyridin-2-yl)amino)thieno[2,3-c]pyridin-2-yl)(3-hydroxyazetidin-1-yl)methanone (A19),
(3-cyclopentyl-5-((5-(piperazin-1-yl)pyridin-2-yl)amino)thieno[2,3-c]pyridin-2-yl)(3-hydroxypyrrolidin-1-yl)methanone (A19a),
(3-cyclobutyl-5-((5-(piperazin-1-yl)pyridin-2-yl)amino)thieno[2,3-c]pyridin-2-yl)(3-hydroxy azetidin-1-yl)methanone (A19b),
(3-cyclopropyl-5-((5-(piperazin-1-yl)pyridin-2-yl)amino)thieno[2,3-c]pyridin-2-yl)(3-hydroxyazetidin-1-yl)methanone (A19c), and
(3-cyclohexyl-5-((5-(piperazin-1-yl)pyridin-2-yl)amino)thieno[2,3-c]pyridin-2-yl)(3-hydroxyazetidin-1-yl)methanone (A19d).

### Embodiment #13

The fused bicyclic compound according to Embodiment #1, which has a Formula (H1), or a pharmaceutically acceptable salt thereof:

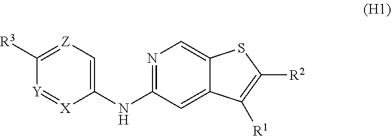

wherein:

X is CH or N;

R¹ is cyclopentyl;

R² is —CN;

R³ is H,

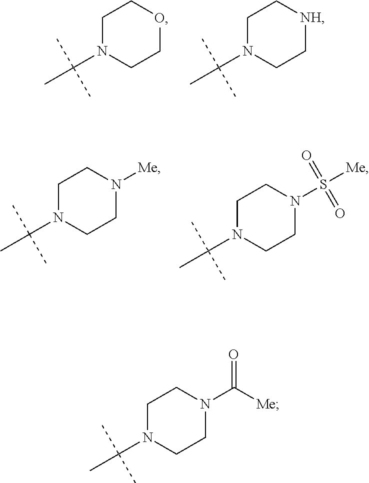

Y is N or CR⁴, wherein R⁴ is H,

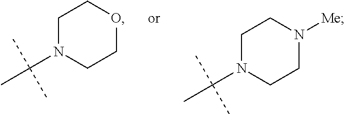

and

Z is CH or N.

### Embodiment #14

The fused bicyclic compound according to Embodiment #1 or a pharmaceutically acceptable salt thereof, wherein the compound is selected from the group consisting of the following compounds:

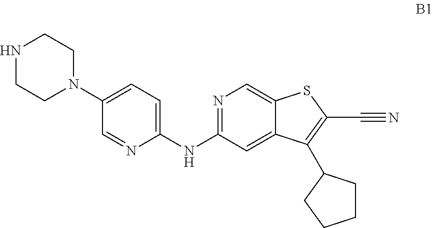 [SMILES: N#Cc1sc2cnc(Nc3ccc(N4CCNCC4)cn3)cc2c1C1CCCC1]

3-cyclopentyl-5-((5-(piperazin-1-yl)pyridin-2-yl)amino)thieno[2,3-c]pyridine-2-carbonitrile (B1),
3-cyclopropyl-5-((5-(piperazin-1-yl)pyridin-2-yl)amino)thieno[2,3-c]pyridine-2-carbonitrile (B1a),
3-cyclobutyl-5-((5-(piperazin-1-yl)pyridin-2-yl)amino)thieno[2,3-c]pyridine-2-carbonitrile (B1b),
3-cyclohexyl-5-((5-(piperazin-1-yl)pyridin-2-yl)amino)thieno[2,3-c]pyridine-2-carbonitrile (B1c),

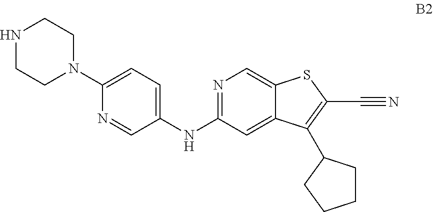 [SMILES: N#Cc1sc2cnc(Nc3ccc(N4CCNCC4)nc3)cc2c1C1CCCC1]

3-cyclopentyl-5-((6-(piperazin-1-yl)pyridin-3-yl)amino)thieno[2,3-c]pyridine-2-carbonitrile (B2),
3-cyclopentyl-5-((6-(4-methylpiperazin-1-yl)pyridin-3-yl)amino)thieno[2,3-c]pyridine-2-carbonitrile (B2a),
3-cyclopropyl-5-((6-(piperazin-1-yl)pyridin-3-yl)amino)thieno[2,3-c]pyridine-2-carbonitrile (B2b),
3-cyclopropyl-5-((6-(4-methylpiperazin-1-yl)pyridin-3-yl)amino)thieno[2,3-c]pyridine-2-carbonitrile (B2c),
3-cyclobutyl-5-((6-(piperazin-1-yl)pyridin-3-yl)amino)thieno[2,3-c]pyridine-2-carbonitrile (B2d),
3-cyclobutyl-5-((6-(4-methylpiperazin-1-yl)pyridin-3-yl)amino)thieno[2,3-c]pyridine-2-carbonitrile (B2e),
3-cyclohexyl-5-((6-(piperazin-1-yl)pyridin-3-yl)amino)thieno[2,3-c]pyridine-2-carbonitrile (B2f),
3-cyclohexyl-5-((6-(4-methylpiperazin-1-yl)pyridin-3-yl)amino)thieno[2,3-c]pyridine-2-carbonitrile (B2g),

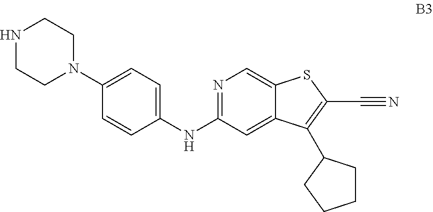 [SMILES: N#Cc1sc2cnc(Nc3ccc(N4CCNCC4)cc3)cc2c1C1CCCC1]

3-cyclopentyl-5-((4-(piperazin-1-yl)phenyl)amino)thieno[2,3-c]pyridine-2-carbonitrile (B3),
3-cyclopropyl-5-((4-(piperazin-1-yl)phenyl)amino)thieno[2,3-c]pyridine-2-carbonitrile (B3a),
3-cyclobutyl-5-((4-(piperazin-1-yl)phenyl)amino)thieno[2,3-c]pyridine-2-carbonitrile (B3b),
3-cyclohexyl-5-((4-(piperazin-1-yl)phenyl)amino)thieno[2,3-c]pyridine-2-carbonitrile (B3c),

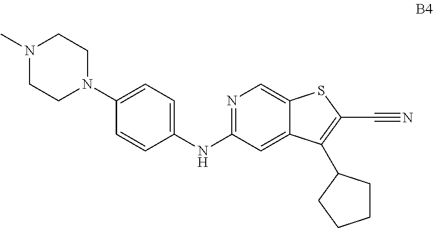 [SMILES: CN1CCN(c2ccc(Nc3cc4c(C5CCCC5)c(C#N)sc4cn3)cc2)CC1]

3-cyclopentyl-5-((4-(4-methylpiperazin-1-yl)phenyl)amino)thieno[2,3-c]pyridine-2-carbonitrile (B4),
3-cyclopropyl-5-((4-(4-methylpiperazin-1-yl)phenyl)amino)thieno[2,3-c]pyridine-2-carbonitrile (B4a),
3-cyclobutyl-5-((4-(4-methylpiperazin-1-yl)phenyl)amino)thieno[2,3-c]pyridine-2-carbonitrile (B4b),
3-cyclohexyl-5-((4-(4-methylpiperazin-1-yl)phenyl)amino)thieno[2,3-c]pyridine-2-carbonitrile (B4c),

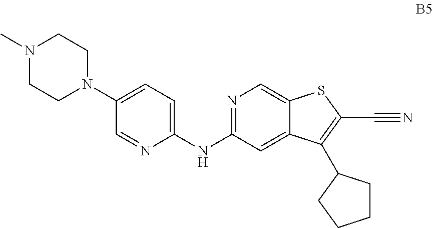 [SMILES: CN1CCN(c2ccc(Nc3cc4c(C5CCCC5)c(C#N)sc4cn3)nc2)CC1]

3-cyclopentyl-5-((5-(4-methylpiperazin-1-yl)pyridin-2-yl)amino)thieno[2,3-c]pyridine-2-carbonitrile (B5),
3-cycloproyl-5-((5-(4-methylpiperazin-1-yl)pyridin-2-yl)amino)thieno[2,3-c]pyridine-2-carbonitrile (B5a),
3-cyclobutyl-5-((5-(4-methylpiperazin-1-yl)pyridin-2-yl)amino)thieno[2,3-c]pyridine-2-carbonitrile (B5b),
3-cyclohexyl-5-((5-(4-methylpiperazin-1-yl)pyridin-2-yl)amino)thieno[2,3-c]pyidine-2-carbonitrile (B5c),

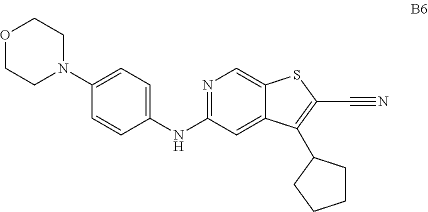 [SMILES: N#Cc1sc2cnc(Nc3ccc(N4CCOCC4)cc3)cc2c1C1CCCC1]

3-cyclopentyl-5-((4-morpholinophenyl)amino)thieno[2,3-c]pyridine-2-carbonitrile (B6),
3-cyclopropyl-5-((4-morpholinophenyl)amino)thieno[2,3-c]pyridine-2-carbonitrile (B6a),
3-cyclobutyl-5-((4-morpholinophenyl)amino)thieno[2,3-c]pyridine-2-carbonitrile (B6b),
3-cyclohexyl-5-((4-morpholinophenyl)amino)thieno[2,3-c]pyridine-2-carbonitrile (B6c),

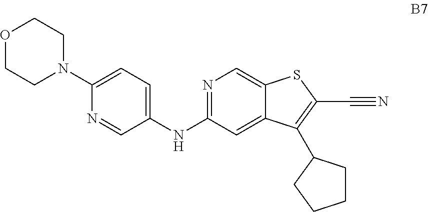 [SMILES: N#Cc1sc2cnc(Nc3ccc(N4CCOCC4)nc3)cc2c1C1CCCC1]

3-cyclopentyl-5-((6-morpholinopyridin-3-yl)amino)thieno[2,3-c]pyridine-2-carbonitrile (B7),
3-cyclopropyl-5-((6-morpholinopyridin-3-yl)amino)thieno[2,3-c]pyridine-2-carbonitrile (B7a),
3-cyclobutyl-5-((6-morpholinopyridin-3-yl)amino)thieno[2,3-c]pyridine-2-carbonitrile (B7b),
3-cyclohexyl-5-((6-morpholinopyridin-3-yl)amino)thieno[2,3-c]pyridine-2-carbonitrile (B7c),

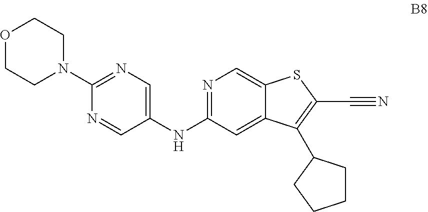 [SMILES: N#Cc1sc2cnc(Nc3cnc(N4CCOCC4)nc3)cc2c1C1CCCC1]

3-cyclopentyl-5-((2-morpholinopyrimidin-5-yl)amino)thieno[2,3-c]pyridine-2-carbonitrile (B8),
3-cyclopropyl-5-((2-morpholinopyrimidin-5-yl)amino)thieno[2,3-c]pyridine-2-carbonitrile (B8a),
3-cyclobutyl-5-((2-morpholinopyrimidin-5-yl)amino)thieno[2,3-c]pyridine-2-carbonitrile (B8b),
3-cyclohexyl-5-((2-morpholinopyrimidin-5-yl)amino)thieno[2,3-c]pyridine-2-carbonitrile (B8c),

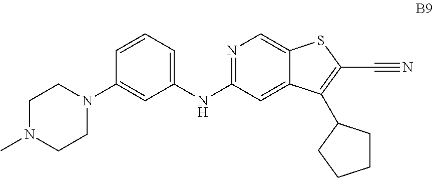 [SMILES: CN1CCN(c2cccc(Nc3cc4c(C5CCCC5)c(C#N)sc4cn3)c2)CC1]

3-cyclopentyl-5-((3-(4-methylpiperazin-1-yl)phenyl)amino)thieno[2,3-c]pyridine-2-carbonitrile (B9),
3-cyclopropyl-5-((3-(4-methylpiperazin-1-yl)phenyl)amino)thieno[2,3-c]pyridine-2-carbonitrile (B9a),
3-cyclobutyl-5-((3-(4-methylpiperazin-1-yl)phenyl)amino)thieno[2,3-c]pyridine-2-carbonitrile (B9b),
3-cyclohexyl-5-((3-(4-methylpiperazin-1-yl)phenyl)amino)thieno[2,3-c]pyridine-2-carbonitrile (B9c),

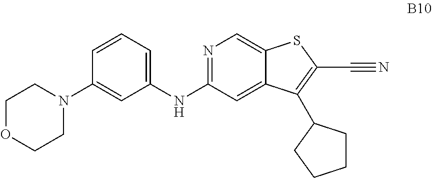 [SMILES: N#Cc1sc2cnc(Nc3cccc(N4CCOCC4)c3)cc2c1C1CCCC1]

3-cyclopentyl-5-((3-morpholinophenyl)amino)thieno[2,3-c]pyridine-2-carbonitrile (B10),
3-cyclopropyl-5-((3-morpholinophenyl)amino)thieno[2,3-c]pyridine-2-carbonitrile (B10a),
3-cyclobutyl-5-((3-morpholinophenyl)amino)thieno[2,3-c]pyridine-2-carbonitrile (B10b),
3-cyclohexyl-5-((3-morpholinophenyl)amino)thieno[2,3-c]pyridine-2-carbonitrile (B10c),

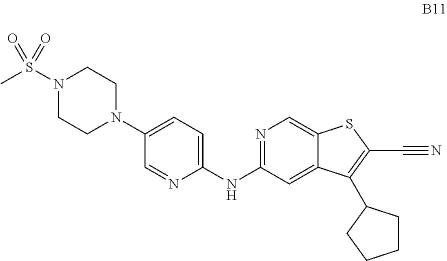 [SMILES: CS(=O)(=O)N1CCN(c2ccc(Nc3cc4c(C5CCCC5)c(C#N)sc4cn3)nc2)CC1]

3-cyclopentyl-5-((5-(4-(methylsulfonyl)piperazin-1-yl)pyridin-2-yl)amino)thieno[2,3-c]pyridine-2-carbonitrile (B11),
3-cyclopropyl-5-((5-(4-(methylsulfonyl)piperazin-1-yl)pyridin-2-yl)amino)thieno[2,3-c]pyridine-2-carbonitrile (B11a),
3-cyclobutyl-5-((5-(4-(methylsulfonyl)piperazin-1-yl)pyridin-2-yl)amino)thieno[2,3-c]pyridine-2-carbonitrile (B11b),
3-cyclohexyl-5-((5-(4-(methylsulfonyl)piperazin-1-yl)pyridin-2-yl)amino)thieno[2,3-c]pyridine-2-carbonitrile (B11c),

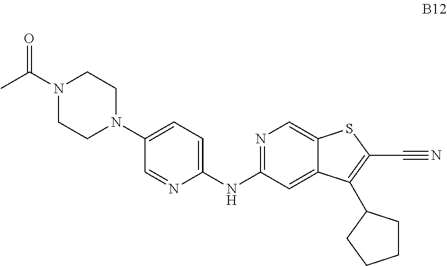 [SMILES: CC(=O)N1CCN(c2ccc(Nc3cc4c(C5CCCC5)c(C#N)sc4cn3)nc2)CC1]

5-((5-(4-acetylpiperazin-1-yl)pyridin-2-yl)amino)-3-cyclopentylthieno[2,3-c]pyridine-2-carbonitrile (B12),
5-((5-(4-acetylpiperazin-1-yl)pyridin-2-yl)amino)-3-cyclopropylthieno[2,3-c]pyridine-2-carbonitrile (B12a),
5-((5-(4-acetylpiperazin-1-yl)pyridin-2-yl)amino)-3-cyclobutylthieno[2,3-c]pyridine-2-carbonitrile (B12b), and
5-((5-(4-acetylpiperazin-1-yl)pyridin-2-yl)amino)-3-cyclohexylthieno[2,3-c]pyridine-2-carbonitrile (B12c).

### Embodiment #15

The pharmaceutically acceptable salt of the fused bicyclic compound according to Embodiment #13, which is

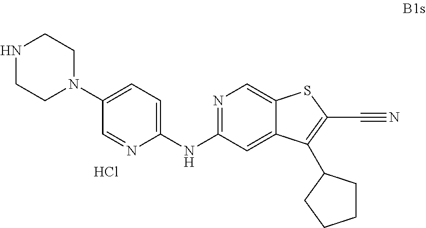 [SMILES: N#Cc1sc2cnc(Nc3ccc(N4CCNCC4)cn3)cc2c1C1CCCC1 ; Cl]

3-cyclopentyl-5-((5-(piperazin-1-yl)pyridin-2-yl)amino)thieno[2,3-c]pyridine-2-carbonitrile hydrochloride (B1s).

### Embodiment #16

A pharmaceutical composition comprising a fused bicyclic compound of anyone of Embodiments #1˜15 or a pharmaceutically acceptable salt thereof, and optionally a pharmaceutically acceptable carrier, vehicle or diluent.

The present invention also provides a pharmaceutical composition comprising a novel compound of Formula H. Accordingly, the invention provides a pharmaceutical composition comprising a therapeutically effective amount of the compound of Formula H or a pharmaceutically acceptable salt thereof, optionally comprising a pharmaceutically acceptable carrier, and optionally comprising at least one additional medicinal or pharmaceutical agent.

The pharmaceutically acceptable carrier may comprise any conventional pharmaceutical carrier or excipient. Suitable pharmaceutical carriers include inert diluents or fillers, water and various organic solvents such as hydrates and solvates. The pharmaceutical compositions may, if desired, contain additional ingredients such as flavorings, binders, excipients and the like. Thus for oral administration, tablets containing various excipients, such as citric acid, may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration, the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may be in a form suitable for oral administration as a tablet, capsule, pill, powder, sustained release formulation, solution or suspension, a form suitable a form suitable for parenteral injection as a sterile solution, suspension or emulsion, a form suitable for topical administration as an ointment or cream, or a form suitable for rectal administration as a suppository. Exemplary parenteral administration forms include solutions or suspensions of active compounds in sterile aqueous solutions, for example, aqueous propylene glycol or dextrose solutions. Such dosage forms may be suitably buffered, if desired.

Oral administration may involve swallowing, so that the compound enters the gastrointestinal tract, and/or buccal, lingual, or sublingual administration by which the compound enters the bloodstream directly from the mouth. Formulations suitable for oral administration include solid, semi-solid and liquid systems such as tablets; soft or hard capsules containing multi- or nano-particulates, liquids, or powders; lozenges (including liquid-filled); chews; gels; fast-dispersing dosage forms; films; ovules; sprays; and buccal/mucoadhesive patches.

Liquid formulations include suspensions, solutions, syrups and elixirs. Such formulations may be employed as fillers in soft or hard capsules (e.g. made from gelatin or hydroxypropyl methyl cellulose) and typically comprise a carrier, for example, water, ethanol, polyethylene glycol, propylene glycol, methyl cellulose, or a suitable oil, and one or more emulsifying agents and/or suspending agents. Liquid formulations may also be prepared by the reconstitution of a solid, for example, from a sachet. The compounds of the invention may also be used in fast-dissolving, fast-disintegrating dosage forms.

For tablet dosage forms, the compound of Formula H may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1 weight % to 25 weight %, for example, from 5 weight % to 20 weight % of the dosage form. Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet. Tablets also generally contain lubricants such as magnesium stearate, calcium stearate, zinc stearate, sodium stearyl fumarate, and mixtures of magnesium stearate with sodium lauryl sulfate. Lubricants generally comprise from 0.25 weight % to 10 weight %, for example, from 0.5 weight % to 3 weight % of the tablet. Other possible ingredients include anti-oxidants, colorants, flavoring agents, preservatives and taste-masking agents. Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt-congealed, or extruded before tabletting. The final formulation may comprise one or more layers and may be coated or uncoated; it may even be encapsulated.

Consumable oral films for human or veterinary use are typically pliable water-soluble or water-swellable thin film dosage forms which may be rapidly dissolving or mucoadhesive and typically comprise a compound of Formula H, a film-forming polymer, a binder, a solvent, a humectant, a plasticizer, a stabilizer or emulsifier, a viscosity-modifying agent and a solvent. The film-forming polymer may be selected from natural polysaccharides, proteins, or synthetic hydrocolloids and is typically present in the range 0.01 to 99 weight %, more typically in the range 30 to 80 weight %. Other possible ingredients include anti-oxidants, colorants, flavorings and flavor enhancers, preservatives, salivary stimulating agents, cooling agents, co-solvents (including oils), emollients, bulking agents, anti-foaming agents, surfactants and taste-masking agents. Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

The compound of Formula H (including pharmaceutically acceptable salts thereof) may also be administered directly into the bloodstream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (for example to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art.

The solubility of compounds of Formula I (including pharmaceutically acceptable salts thereof) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compound of Formula H may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(DL-lactic-coglycolic acid) (PLGA) microspheres.

The compound of Formula H (including pharmaceutically acceptable salts thereof) may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated. Other means of topical administration include delivery by electroporation, iontophoresis, phonophoresis, sonophoresis and microneedle or needle-free injection. Formulations for topical administration may be formulated to be immediate and/or modified release. Modified release formulations include delayed-, sustained-, pulsed-, controlled-, targeted and programmed release.

The compound of Formula H (including pharmaceutically acceptable salts thereof) can also be administered intranasally or by inhalation, typically in the form of a dry powder (either alone; as a mixture, for example, in a dry blend with lactose; or as a mixed component particle, for example, mixed with phospholipids, such as phosphatidylcholine) from a dry powder inhaler, as an aerosol spray from a pressurized container, pump, spray, atomizer (for example an atomizer using electrohydrodynamics to produce a fine mist), or nebulizer, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurized container, pump, spray, atomizer, or nebulizer contains a solution or suspension of the compound of Formula H comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid. Prior to use in a dry powder or suspension formulation, the drug product is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

Capsules (made from gelatin or hydroxypropyl methyl cellulose), blisters and cartridges for use in an inhaler or insufflator may be formulated to contain a powder mix of the compound of Formula H, a suitable powder base such as lactose or starch and a performance modifier such as L-leucine, mannitol, or magnesium stearate. The lactose may be anhydrous or in the form of the monohydrate. Other suitable excipients include dextran, glucose, maltose, sorbitol, xylitol, fructose, sucrose and trehalose. A suitable solution formulation for use in an atomizer using electrohydrodynamics to produce a fine mist may contain from 1 μg to 20 mg of the compound of the invention per actuation and the actuation volume may vary from 1 μL to 100 μL. A typical formulation may comprise a compound of Formula H or a pharmaceutically acceptable salt thereof, propylene glycol, sterile water, ethanol and sodium chloride. Alternative solvents which may be used instead of propylene glycol include glycerol and polyethylene glycol. Suitable flavors, such as menthol and levomenthol, or sweeteners, such as saccharin or saccharin sodium, may be added to those formulations of the invention intended for inhaled/intranasal administration. Formulations for inhaled/intranasal administration may be formulated to be immediate and/or modified release using, for example, PGLA. Modified release formulations include delayed-, sustained-, pulsed-, controlled-, targeted and programmed release.

The compound of Formula H (including pharmaceutically acceptable salts thereof) may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release. Modified release formulations include delayed-, sustained-, pulsed-, controlled-, targeted and programmed release.

The compound of Formula H (including pharmaceutically acceptable salts thereof) may also be administered directly to the eye or ear, typically in the form of drops of a micronized suspension or solution in isotonic, pH-adjusted, sterile saline. Other formulations suitable for ocular and aural administration include ointments, gels, biodegradable (e.g., absorbable gel sponges, collagen) and non-biodegradable (e.g., silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropyl methyl cellulose, hydroxyethyl cellulose, or methyl cellulose, or a heteropolysaccharide polymer, for example, gelan gum, may be incorporated together with a preservative, such as benzalkonium chloride. Such formulations may also be delivered by iontophoresis. Formulations for ocular/aural administration may be formulated to be immediate and/or modified release. Modified release formulations include delayed-, sustained-, pulsed-, controlled-, targeted, or programmed release.

The compound of Formula H (including pharmaceutically acceptable salts thereof) may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e., as a carrier, diluent, or solubilizer.

The pharmaceutical composition may be in unit dosage forms suitable for single administration of precise dosages. One of ordinary skill in the art would appreciate that the composition may be formulated in sub-therapeutic dosage such that multiple doses are envisioned.

### Embodiment #17

A method of synthesizing a compound of formula (H1),

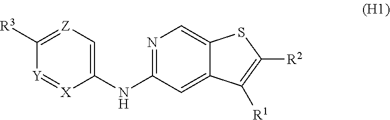

wherein:

X is CH or N;

R¹ is methyl, isopropyl, cyclopropyl, cyclobutyl, cyclopentyl, cyclohexyl, 1-methoxyl cyclopentyl, 1-cyclopentenyl, 3-bicyclo[3.1.0]hexyl, tetrahydrofuran-3-yl, or 3,3-difluorocyclopentyl;

R² is —CN,

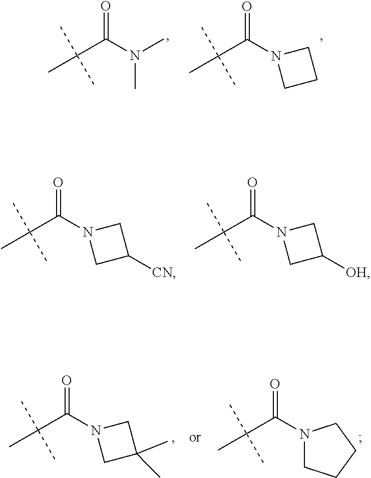

R³ is H,

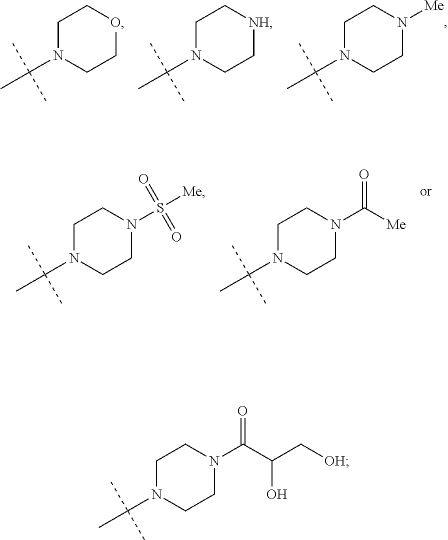

Y is N or CR⁴, wherein R⁴ is H,

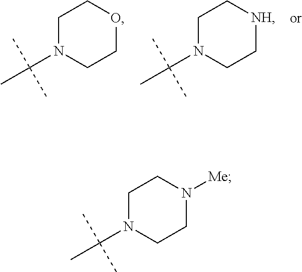

and

Z is CH or N;

the method including:

(1) reacting a compound of formula V1 with a compound of formula V2 to produce compound of formula V3,

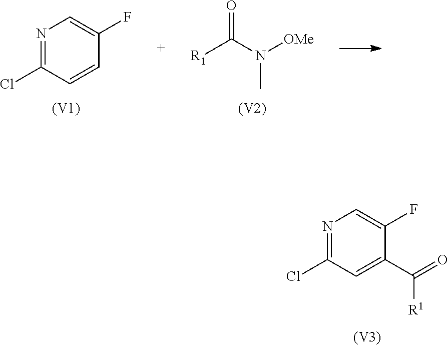

(2) converting the compound of formula V3 to a compound of formula V4,

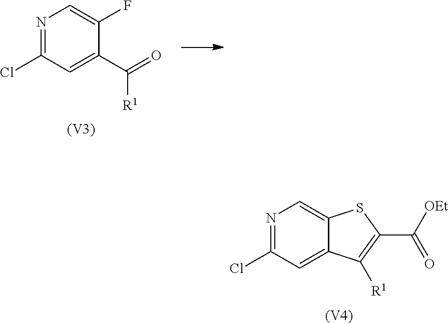

(3) converting the compound of formula V4 to a compound of formula V5,

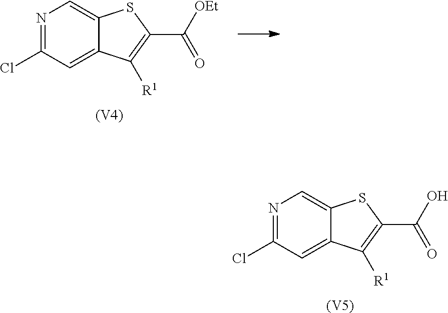

(4) converting the compound of formula V5 to a compound of formula V6, and

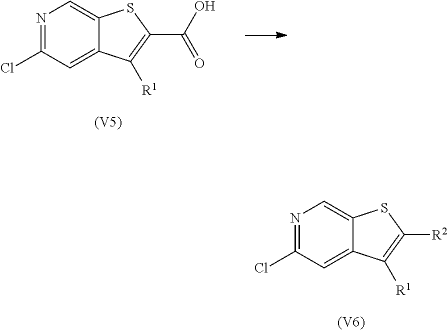

(5) reacting the compound of formula V6 with a compound of formula V7 to produce compound of formula (H1):

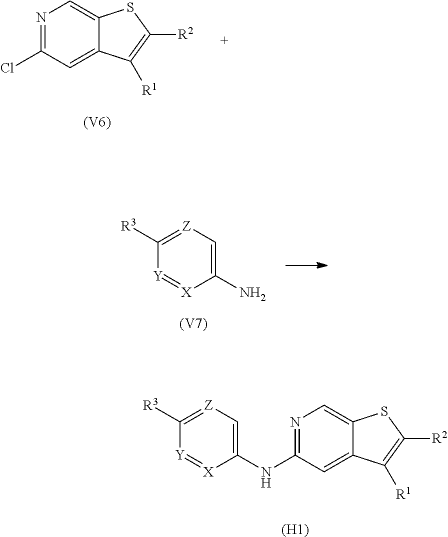

Starting materials and intermediates useful for making the compounds of the present invention can be obtained from chemical vendors or can be made according to methods described in the chemical art.

Compounds of the invention, including salts of the compounds, can be prepared using known organic synthesis techniques and can be synthesized according to any of numerous possible synthetic routes. The reactions for preparing compounds of the invention can be carried out in suitable solvents, which can be readily selected by one of skill in the art of organic synthesis. Suitable solvents can be substantially non-reactive with the starting materials (reactants), the intermediates, or products at the temperatures at which the reactions are carried out, e.g., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention may involve the protection and deprotection of various chemical groups. The need for protection and deprotection, and the selection of appropriate protecting groups, can be readily determined by one skilled in the art. For example, a —CN group can be hydrolyzed to afford an amide group; a carboxylic acid can be converted to an amide; a carboxylic acid can be converted to an ester, which in turn can be reduced to an alcohol, which in turn can be further modified. For another example, an OH group can be converted into a better leaving group such as a methanesulfonate, which in turn is suitable for nucleophilic substitution, such as by a cyanide ion. For another example, an —S— can be oxidized to —S(═O)— and/or —S(═O)₂—. For yet another example, an unsaturated bond such as C═C double bond or C—C triple bond can be reduced to a saturated bond by hydrogenation.

Functional (reactive) groups can be protected/deprotected in the course of the synthetic scheme, if appropriate and/or desired. For example, an OH group can be protected by a benzyl, methyl, or acetyl group, which can be deprotected and converted back to the OH group in a later stage of the synthetic process. For another example, an NH₂ group can be protected by a benzyloxycarbonyl (Cbz) or BOC group; conversion back to the NH₂ group can be carried out at a later stage of the synthetic process via deprotection.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high-performance liquid chromatography (HPLC) or thin layer chromatography (TLC). For example, compounds of Formula I in Embodiment #9 and intermediates thereof may be prepared according to the reaction scheme as shown in Embodiment #17.

In some embodiments, the compounds may exist as stereoisomers, such as atropisomers, racemates, enantiomers, or diastereomers. Conventional techniques for the preparation/isolation of individual enantiomers include chiral synthesis from a suitable optically pure precursor or resolution of the racemate using, for example, chiral high-performance liquid chromatography (HPLC). Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomeric mixture may be separated by chromatography and/or fractional crystallization, and one or both of the diastereoisomers converted to the corresponding pure enantiomer(s) by means well known to one skilled in the art. Chiral compounds (and chiral precursors thereof) may be obtained in enantiomerically enriched form using chromatography, typically HPLC, on an asymmetric resin with a mobile phase consisting of a hydrocarbon, typically heptane or hexane, containing from 0% to 50% 2-propanol, typically from 2% to 20%, and from 0% to 5% of an alkylamine, typically 0.1% diethylamine. Concentration of the eluate affords the enriched mixture. Stereoisomeric conglomerates may be separated by conventional techniques known to those skilled in the art. Suitable stereoselective techniques are well known to those of ordinary skill in the art. For a compound of Formula H that contains an alkenyl or alkenylene group, geometric cis/trans (or Z/E) isomers are possible. Cis/trans isomers may be separated by conventional techniques well known to those skilled in the art, for example, chromatography and fractional crystallization.

Salts of the present invention can be prepared according to methods known to those of skill in the art. Basic compounds are capable of forming a wide variety of salts with various inorganic and organic acids that are pharmaceutically acceptable for administration to animals. Alternatively, one can initially isolate the compound of the present invention from the reaction mixture as a pharmaceutically unacceptable salt, and then simply convert the latter back to the free base compound by treatment with an alkaline reagent. Subsequently, he or she converts the latter free base to a pharmaceutically acceptable acid addition salt. The acid addition salts of the basic compounds of this invention can be prepared by treating the basic compound with a substantially equivalent amount of the selected mineral or organic acid in an aqueous solvent medium or in a suitable organic solvent, such as methanol or ethanol. Upon evaporation of the solvent, the desired solid salt is obtained. The desired acid salt can also be precipitated from a solution of the free base in an organic solvent by adding an appropriate mineral or organic acid to the solution.

For the compound of Formula H that is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, isonicotinic acid, lactic acid, pantothenic acid, bitartric acid, ascorbic acid, 2,5-dihydroxybenzoic acid, gluconic acid, saccharic acid, formic acid, methanesulfonic acid, ethanesulfonic acid, benzenesulfonic acid, p-toluenesulfonic acid, and pamoic [i.e., 4,4′-methanediylbis(3-hydroxynaphthalene-2-carboxylic acid)] acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as ethanesulfonic acid, or the like.

For compounds of Formula H that are acidic in nature, their base salts can be formed with various pharmacologically acceptable cations. Examples of such salts include the alkali metal or alkaline earth metal salts, and particularly the sodium and potassium salts. These salts are all prepared by conventional techniques. The chemical bases which are used as reagents to prepare the pharmaceutically acceptable base salts of this invention are those which form non-toxic base salts with the acidic compounds of Formula H. These salts may be prepared by any suitable method, for example, treatment of the free acid with an inorganic or organic base, such as an amine (primary, secondary or tertiary), an alkali metal hydroxide or alkaline earth metal hydroxide, or the like. These salts can also be prepared by treating the corresponding acidic compounds with an aqueous solution containing the desired pharmacologically acceptable cations, and then evaporating the resulting solution to dryness, for example under reduced pressure. Alternatively, they may also be prepared by mixing lower alkanolic solutions of the acidic compounds and the desired alkali metal alkoxide together, and then evaporating the resulting solution to dryness in the same manner. In either case, stoichiometric quantities of reagents are, for example, employed in order to ensure completeness of reaction and maximum yields of the desired final product.

Pharmaceutically acceptable salts of compounds of Formula H may be prepared by, e.g., one or more of three methods: (i) by reacting the compound of Formula H with the desired acid or base; (ii) by removing an acid- or base-labile protecting group from a suitable precursor of the compound of Formula H or by ring-opening a suitable cyclic precursor, for example, a lactone or lactam, using the desired acid or base; or (iii) by converting one salt of the compound of Formula H to another by reaction with an appropriate acid or base or by means of a suitable ion exchange column. All three reactions are typically carried out in solution. The resulting salt may precipitate out and be collected by filtration or may be recovered by evaporation of the solvent. The degree of ionization in the resulting salt may vary from completely ionized to almost non-ionized.

### Embodiment #18

A method for treating a CDK-mediated disease or disorder in a mammal, which method comprises administering to said mammal a therapeutically effective amount of a fused bicyclic compound of anyone of Embodiments #1˜#14 or a pharmaceutically acceptable salt thereof.

The term “therapeutically effective amount” as used herein refers to that amount of the compound (including a pharmaceutically acceptable salt thereof) being administered which will relieve to some extent one or more of the symptoms of the disorder being treated. In reference to the treatment of a CDK-mediated disease or disorder, a therapeutically effective amount refers to that amount which has the effect of relieving to some extent or eliminating one or more symptoms associated with the CDK-mediated disease or disorder. The term “treating”, as used herein, unless otherwise indicated, means reversing, alleviating, inhibiting the progress of, or preventing the disorder or condition to which such term applies, or one or more symptoms of such disorder or condition. The term “treating” also includes adjuvant and neo-adjuvant treatment of a subject.

The invention also relates to prodrugs of the compounds of Formula H. Some compounds of Formula H may have little or no pharmacological activity themselves, but they can, when administered into or onto the body, be converted into compounds of Formula H having the desired activity, for example, by hydrolytic cleavage. Such derivatives are referred to as “prodrugs”. Prodrugs in accordance with the invention can, for example, be produced by replacing appropriate functionalities present in the compounds of Formula H with certain moieties known to those skilled in the art as “pro-moieties”. In some embodiments, certain compounds of Formula H may themselves act as prodrugs of other compounds of Formula H. Metabolites of compounds of Formula H formed in vivo upon administration of the drug are also included within the scope of the invention.

Administration of the compounds of Formula H (including salts thereof) may be effected by any method that enables delivery of the compounds to the site of action. These methods include, for example, enteral routes (e.g., oral routes, buccal routes, sublabial routes, sublingual routes), oral routes, intranasal routes, inhaled routes, intraduodenal routes, parenteral injection (including intravenous, subcutaneous, intramuscular, intravascular or infusion), intrathecal routes, epidural routes, intracerebral routes, intracerbroventricular routes, topical, and rectal administration. In one embodiment of the present invention, the compounds of Formula H may be administered/effected by parenteral injection routes (e.g., intravenous injection route). In one embodiment of the present invention, the compounds of Formula H may be administered or effected by oral routes.

Dosage of the compounds of Formula H may be adjusted to provide the desired response. For example, a single bolus may be administered, several divided doses may be administered over time, or the dose may be proportionally reduced or increased as indicated by the exigencies of the therapeutic situation. It may be advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specifications for the dosage unit forms of the invention are dictated by a variety of factors such as the unique characteristics of the therapeutic agent and the particular therapeutic or prophylactic effect to be achieved.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent is well-known in the relevant art and would be understood to be encompassed by the skilled artisan once provided the teachings disclosed herein.

The administered amount of the compound of Formula H will be dependent on the subject being treated, the severity of the disorder or condition, the rate of administration, the disposition of the compound and the discretion of the prescribing physician. Generally, an effective dosage is in the range of about 0.0001 to about 50 mg per kg body weight per day. In some embodiments, dosage levels below the lower limit of the aforesaid range may be more than adequate, while in other cases still larger doses may be employed without causing any harmful side effect.

The administration of a compound of Formula H or a pharmaceutically acceptable salt thereof together with an at least one additional pharmaceutical or medicinal agent, either sequentially or simultaneously, may be carried out for combination therapy. Accordingly, the present invention also includes pharmaceutical compositions comprising (a) a first agent comprising a compound of Formula H (including a pharmaceutically acceptable salt thereof); (b) a second pharmaceutically active agent; and (c) a pharmaceutically acceptable carrier, vehicle or diluent. The second pharmaceutically active agents may be selected for use in conjunction with the compounds of Formula H, depending on the disease, disorder, or condition to be treated.

### Embodiment #19

The method according to Embodiment #18, wherein said CDK is CDK1, CDK2, CDK4, CDK5, CDK6, CDK7, CDK9, or any combination thereof.

### Embodiment #20

The method according to Embodiment #18, wherein said CDK-mediated disease or disorder is selected from cancer, brain tumor, and autoimmune diseases.

### Embodiment #21

A method for inhibiting CDK comprising contacting the CKD with a fused bicyclic compound of anyone of Embodiments #1˜14 or a pharmaceutically acceptable salt thereof. The step of inhibiting may be carried out in vitro or in vivo. “In vitro” refers to procedures performed in an artificial environment such as, e.g., without limitation, in a test tube or culture medium. “In vivo” refers to procedures performed within a living organism such as, without limitation, a human, a mouse, rat or rabbit.

Some compounds of the present invention possess highly potent and selective CDK kinase inhibitory activity and could be useful in reducing and inhibiting CDK enzymatic activity in various types of cells and the treatment of diseases and disorders mediated by CDKs, such as cancers e.g. brain cancer.

### Embodiment #22

Use of a fused bicyclic compound of anyone of Embodiments #1˜14 or a pharmaceutically acceptable salt thereof in the manufacturing of a medicament for treating a CDK-mediated disease or disorder.

The invention will be described in greater detail by way of specific examples. The following examples are offered for illustrative purposes, and are not intended to limit the invention in any manner. Those of skill in the art will readily recognize a variety of non-critical parameters that can be changed or modified to yield essentially the same results. Additional compounds within the scope of this invention may be prepared using the methods illustrated in these Examples, either alone or in combination with techniques generally known in the art.

### EXAMPLES

The following illustrate the synthesis of various compounds of the present invention. Additional compounds within the scope of this invention may be prepared using the methods illustrated in these Examples, either alone or in combination with techniques generally known in the art.

### Example 1: Synthesis of Compound of Formula A1 in Embodiment #12

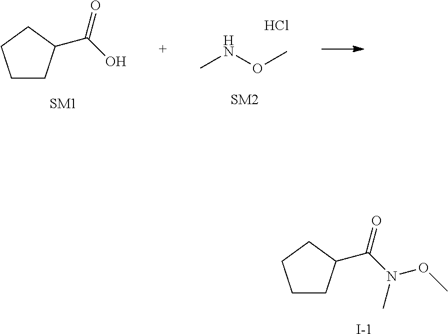

To a 100 mL flask was added SM1 (5.00 g, 43.8 mmol, 1.0 eq), acetonitrile (MeCN, 25 mL), N,N-Diisopropylethylamine (DIPEA, 11.32 g, 87.6 mmol, 2.0 eq) and [benzotriazol-1-yloxy(dimethylamino)methylidene]-dimethylazanium tetrafluoroborate (TBTU, 14.76 g, 46.0 mmol, 1.05 eq) under nitrogen gas atmosphere at ambient temperature, and a pre-mixed solution of SM2 (5.13 g, 52.6 mmol, 1.2 eq) and DIPEA (5.66 g, 43.8 mmol, 1.0 eq) in MeCN (25 mL) was added. The resultant mixture was stirred at ambient temperature for 12 hours. The reaction mixture was concentrated to dryness, and was treated with ethyl acetate (EA, 50 mL) and 0.1 N aqueous NaOH (100 mL). Organic phase was separated, and washed with 0.1 N aqueous HCl (100 mL), dried over MgSO₄, and was concentrated to dryness. The obtained residue was purified by a column chromatography (petroleum ether (PE)/EA=3/1) to afford compound I-1 (4.80 g, 84% yield) as a colorless liquid.

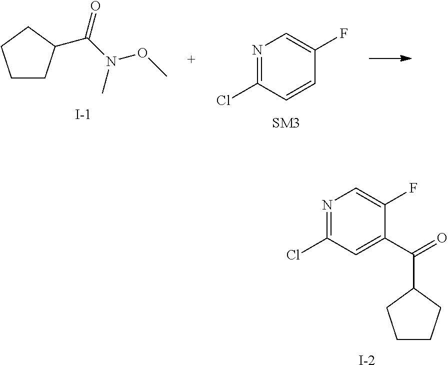

To a 250 mL flask was added SM3 (2.00 g, 19.8 mmol, 1.3 eq) and tetrahydrofuran (THF, 20 mL), and a solution of n-butyllithium (BuLi, 2.5 N in hexane, 7.3 mL) was added dropwise under nitrogen gas atmosphere at −78° C., and the resultant mixture was stirred for additional 1 hour. A solution of I-1 (3.11 g, 15.2 mmol, 1.0 eq) in THF was added, and the mixture was warmed to ambient temperature. The resultant mixture was stirred at ambient temperature for 10 hours, and was quenched with saturated NH₄Cl. Organic phase was separated, dried over MgSO₄, and was concentrated to dryness. The obtained residue was purified by a column chromatography (PE/EA=15/1) to afford compound I-2 (2.10 g, 61% yield) as a colorless oil.

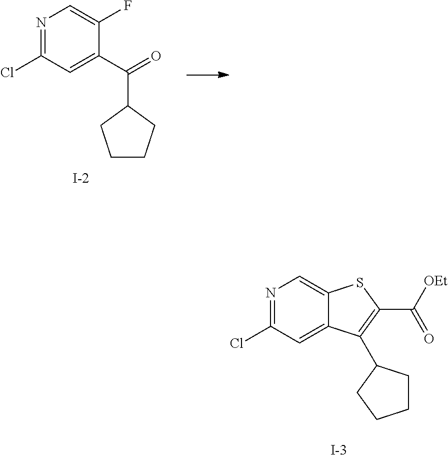

To a 25 mL flask was added I-2 (0.50 g, 2.2 mmol, 1.0 eq), ethyl thioglycolate (0.32 g, 2.6 mmol, 1.2 eq) and THF (5 mL) cooled with an ice-water bath under nitrogen atmosphere, and NaH (60% in oil, 0.18 g, 4.4 mmol, 1.0 eq) was added in portions. The resultant mixture was heated to reflux for additional 12 hours. The mixture was cooled to ambient temperature, and was poured into cold water. The aqueous phase was extracted with ethyl acetate, washed with brine, dried over MgSO₄, and was concentrated to dryness. The obtained residue was purified by a column chromatography (PE/EA=10/1) to afford compound I-3 (0.46 g, 68% yield) as a yellow oil.

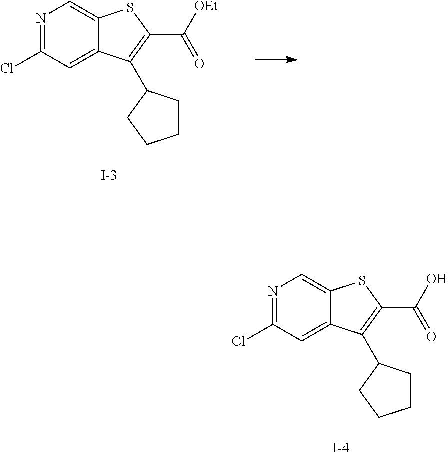

To a 25 mL flask was added I-3 (46 mg, 0.15 mmol, 1.0 eq), LiOH (50 mg), water (1 mL) and THF (3 mL), and the resultant mixture was stirred at ambient temperature for 12 hours. The reaction mixture was concentrated to dryness, and was treated with water and dichloromethane (DCM). Organic phase was discarded, and aqueous phase was treated with 6 N aqueous HCl until pH=2 was achieved. Precipitate was collected by filtration, and was dried to afford compound I-4 (35 mg, 83% yield) as a white powder.

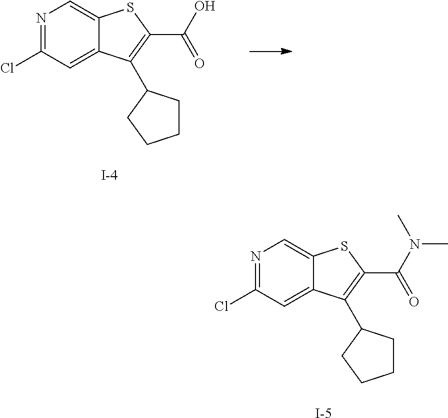

To a 50 mL flask was added I-4 (35 mg, 0.12 mmol, 1.0 eq), carbonyldiimidazole (CDI, 23 mg, 0.14 mmol, 1.1 eq) and THF (3 mL), and the obtained mixture was heated to reflux for 3 hours under nitrogen gas atmosphere. The reaction was cooled to ambient temperature, and a solution of dimethylamine (2.0 N, 1 mL) was added. The resultant mixture was stirred at ambient temperature for additional 12 hours. The reaction mixture was concentrated to dryness, and the obtained residue was purified by a column chromatography (PE/EA=5/1) to afford compound I-5 (40 mg, 100% yield) as an off-white solid.

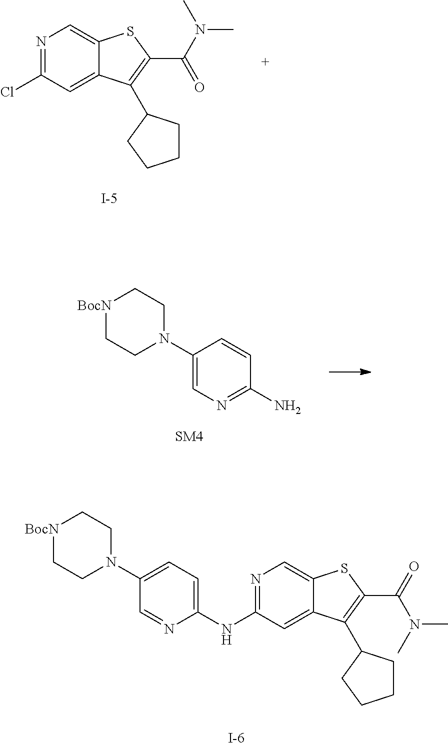

To a 50 mL flask was added I-5 (40 mg, 0.12 mmol, 1.0 eq), SM4 (40 mg, 0.14 mmol, 1.2 eq), Cs₂CO₃ (117 mg, 0.36 mmol, 3.0 eq), tris(dibenzylideneacetone)dipalladium(0) (Pd₂(dba)₃, catalytic amount), 4,5-bis(diphenylphosphino)-9,9-dimethylxanthene (Xantphos, catalytic amount) and 1,4-dioxane (10 mL), and the resultant mixture was heated to reflux for 12 hours under nitrogen gas atmosphere. The reaction mixture was concentrated to dryness, and the obtained residue was purified by a column chromatography (DCM/MeOH=50/1) to afford I-6 (80 mg, 100% yield) as a light yellow solid.

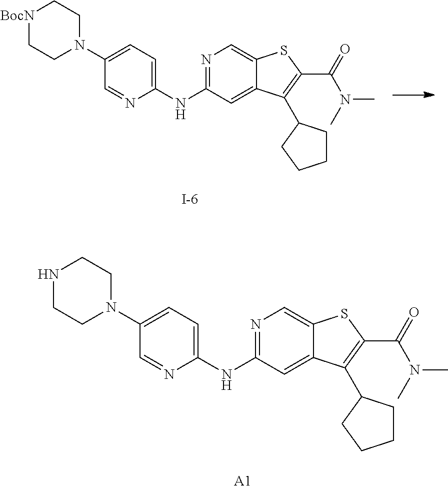

To a 100 mL flask was added I-6 (80 mg, 0.12 mmol, 1.0 eq), TFA (3 mL) and DCM (10 mL), and the resultant mixture was stirred at ambient temperature 12 hours. The reaction mixture was concentrated to dryness, and the residue was treated with DCM and saturated Na₂CO₃. Organic phase was separated, dried over MgSO₄, and was concentrated to dryness. The crude product was treated with methyl tert-butyl ether (MTBE), and was filtered to afford compound A1 (5.8 mg, 11% yield) as a yellow solid. ¹H NMR (400 MHz, DMSO-d₆) δ(ppm) 12.45 (br, 1H), 9.41 (br, 1H), 9.16 (s, 1H), 8.12-8.15 (dd, 1H), 7.85-7.86 (d, 1H), 7.81 (s, 1H), 7.51-7.54 (d, 1H), 3.59-3.62 (m, 4H), 3.21-3.40 (m, 5H), 3.05 (s, 3H), 2.93 (s, 3H), 1.99 (brs, 4H), 1.76-1.87 (m, 4H). LC-MS(m/z): 451 (M+H⁺).

### Example 2: Synthesis of Compound A6

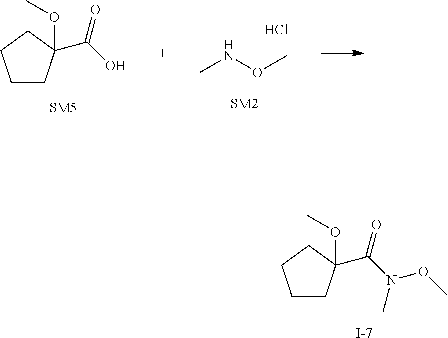

Compound I-7 was synthesized in the same method as that for compound I-1, using SM5 as starting material and with an 80% yield.

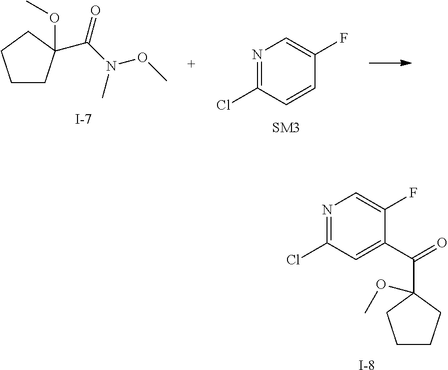

Compound I-8 was synthesized in the same method as that for compound I-2, using I-7 as starting material and with a 62% yield.

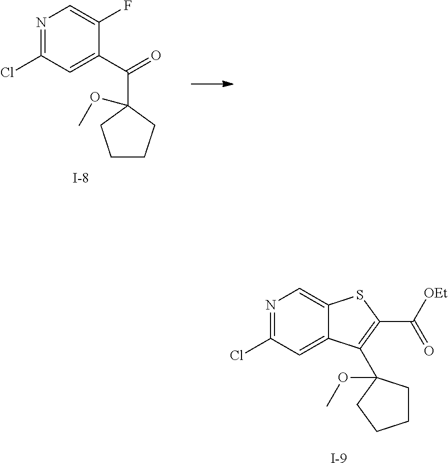

Compound I-9 was synthesized in the same method as that for compound I-3, using I-8 as starting material and with a 100/% yield.

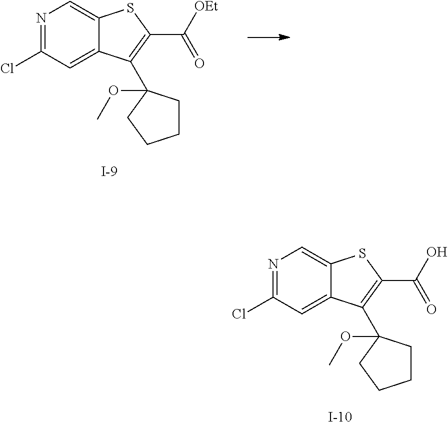

Compound I-10 was synthesized in the same method as that for compound I-4, using I-9 as starting material and with an 81% yield.

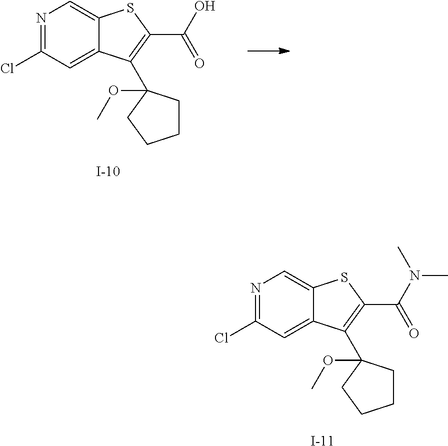

Compound I-11 was synthesized in the same method as that for compound I-5, using I-10 as starting material and with a 95% yield.

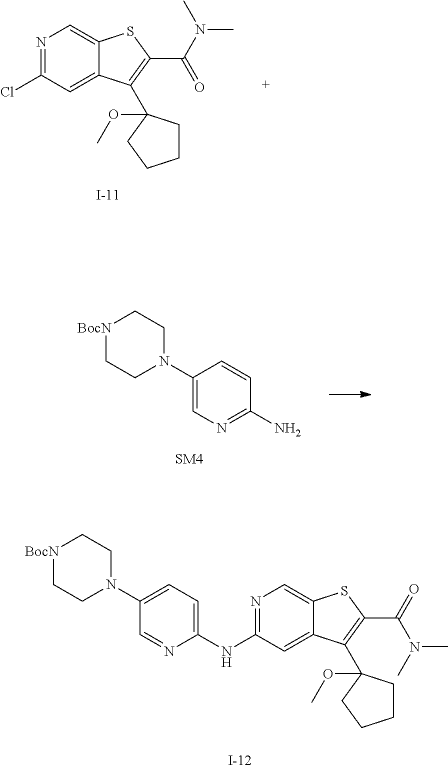

Compound I-12 was synthesized in the same method as that for compound I-6, using I-11 as starting material and with a 99% yield.

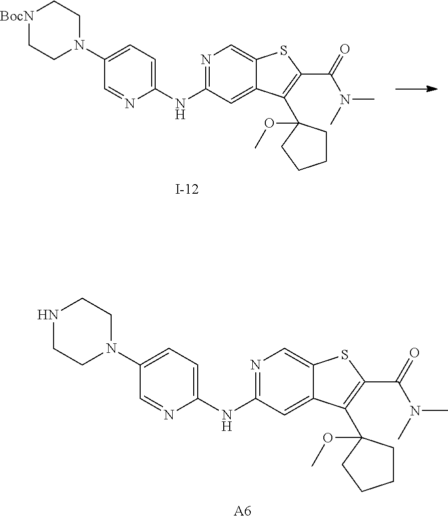

Compound A6 was synthesized in the same method as that for compound A1, using I-12 as starting material and with a 12% yield. ¹H NMR (400 MHz, DMSO-d₆) δ(ppm) 9.44 (s, 1H), 8.84 (s, 1H), 8.42 (s, 1H), 7.90-7.91 (d, 1H), 7.39-7.49 (m, 2H), 6.14 (s, 1H), 2.88-3.08 (m, 14H), 2.65-2.67 (m, 2H), 1.98-2.02 (m, 2H). LC-MS(m/z): 481 (M+H⁺).

### Example 3: Synthesis of Compound A7

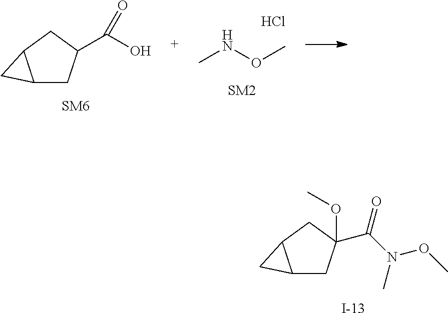

Compound I-13 was synthesized in the same method as that for compound I-1, using SM6 as starting material and with an 85% yield.

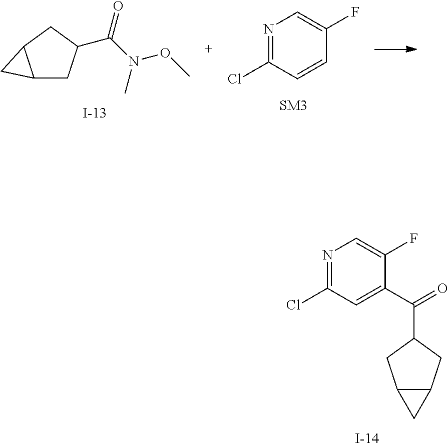

Compound I-14 was synthesized in the same method as that for compound I-2, using I-13 as starting material and with an 85% yield.

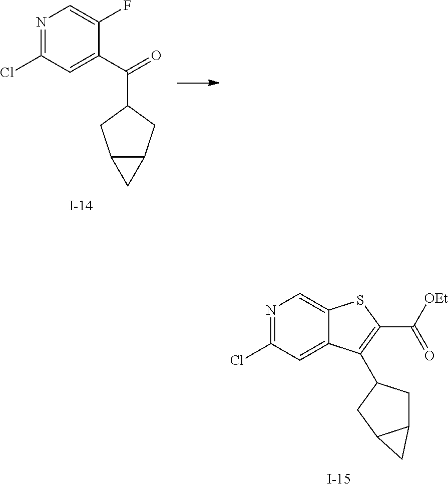

Compound I-15 was synthesized in the same method as that for compound I-3, using I-14 as starting material and with an 11% yield.

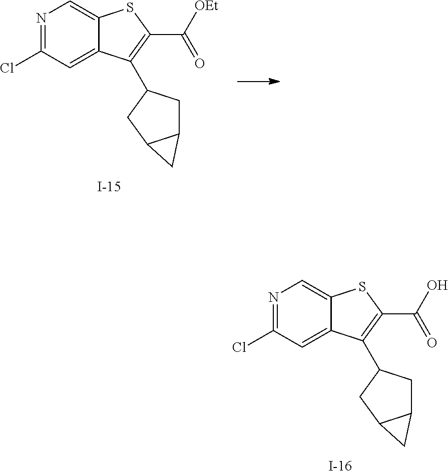

Compound I-16 was synthesized in the same method as that for compound I-4, using I-15 as starting material and with an 86% yield.

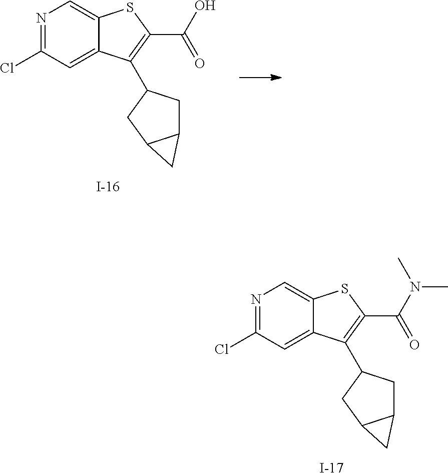

Compound I-17 was synthesized in the same method as that for compound I-5, using I-16 as starting material and with a 98% yield.

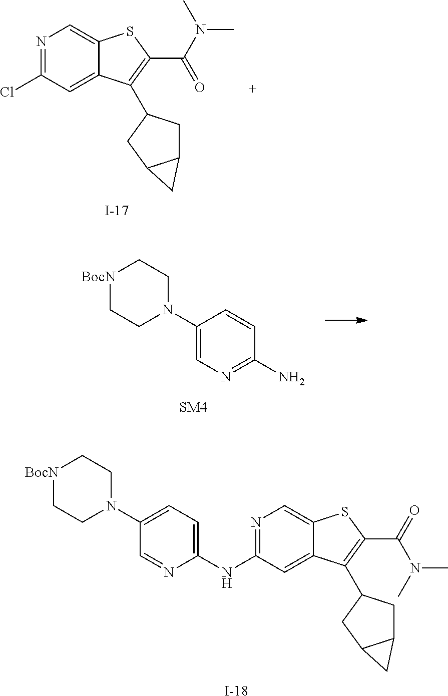

Compound I-18 was synthesized in the same method as that for compound I-6, using I-17 as starting material and with a 96% yield.

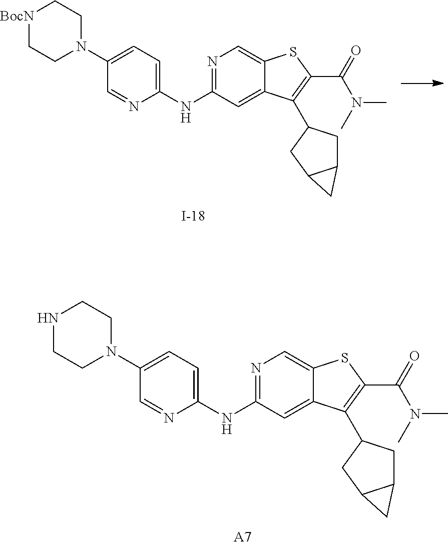

Compound A7 was synthesized in the same method as that for compound A1, using I-18 as starting material and with an 11% yield. ¹H NMR (400 MHz, DMSO-d₆) δ(ppm) 9.53 (s, 1H), 8.41 (s, 1H), 7.92 (s, 1H), 7.43-7.48 (m, 2H), 2.90-3.25 (m, 14H), 2.25-2.31 (m, 2H), 1.93-1.98 (m, 2H), 1.52-1.53 (d, 2H), 0.50 (m, 1H), 0.48 (m, 1H). LC-MS(m/z): 463 (M+H⁺).

### Example 4: Synthesis of Compound A10

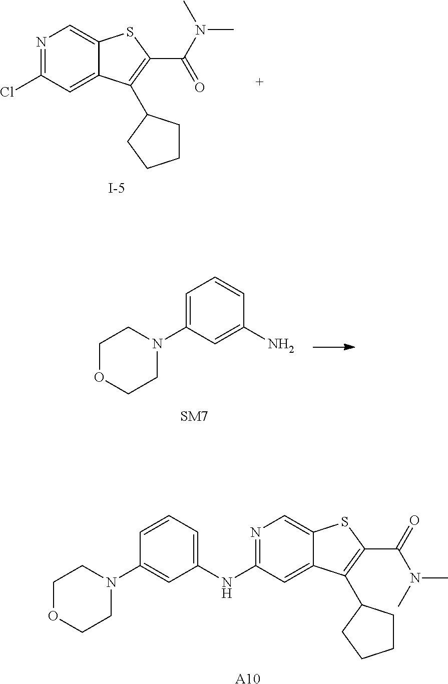

Compound A10 was synthesized in the same method as that for compound I-6, using SM7 as starting material and with a 45% yield. ¹H NMR (400 MHz, DMSO-d₆) δ(ppm) 8.88 (s, 1H), 8.82 (s, 1H), 7.24 (s, 1H), 7.07-7.15 (m, 3H), 7.50-7.52 (m, 1H), 3.73-3.76 (m, 4H), 3.15-3.19 (m, 5H), 3.02 (s, 3H), 2.92 (s, 3H), 1.69-1.96 (m, 8H). LC-MS(m/z): 451 (M+H⁺).

### Example 5: Synthesis of Compound A11

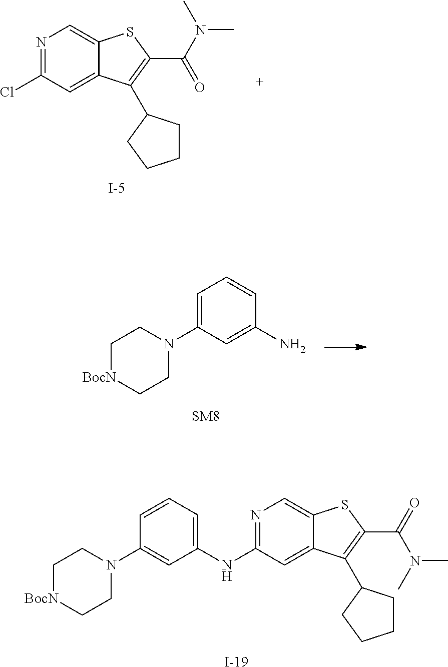

Compound I-19 was synthesized in the same method as that for compound I-6, using SM8 as starting material and with a 97% yield.

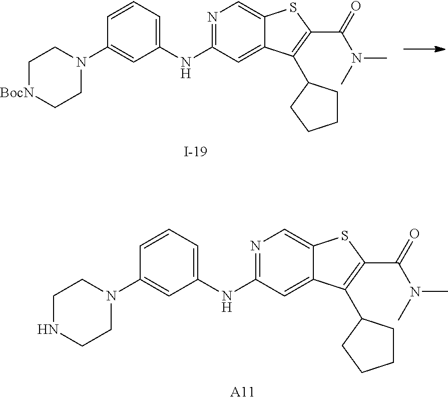

Compound A11 was synthesized in the same method as that for compound A1, using I-19 as starting material and with a 15% yield. ¹H NMR (400 MHz, DMSO-d₆) δ(ppm) 8.86 (s, 1H), 8.82 (s, 1H), 7.24 (s, 1H), 7.05-7.14 (m, 3H), 7.49-7.51 (d, 1H), 3.02-3.22 (m, 12H), 1.719-1.96 (m, 8H). LC-MS(m/z): 450 (M+H⁺).

### Example 6: Synthesis of Compound A12

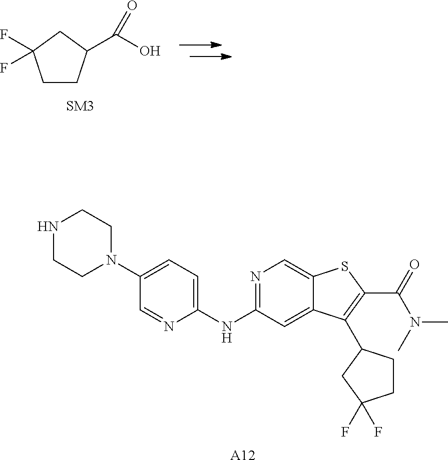 [SMILES: O=C(O)C1CCC(F)(F)C1 ; CN(C)C(=O)c1sc2cnc(Nc3ccc(N4CCNCC4)cn3)cc2c1C1CCC(F)(F)C1 ; "A12"]

Compound A12 was synthesized in the same method as that for compound A1 in Example 1, using 3,3-difluorocyclopentanecarboxylic acid SM3 which is commercially available from PharmaBlock, Nanjing, China. ¹HNMR (400 MHz, DMSO-d₆) δ(ppm) 8.96 (s, 1H), 8.69 (s, 1H), 8.33 (brs, 1H), 7.90 (s, 1H), 7.62 (br, 1H), 7.41 (d, 1H), 3.05 (s, 3H), 2.94 (s, 3H), 2.16-2.33 (m, 4H). LC-MS(m/z):487 (M+H⁺).

### Example 7: Synthesis of Compound A13

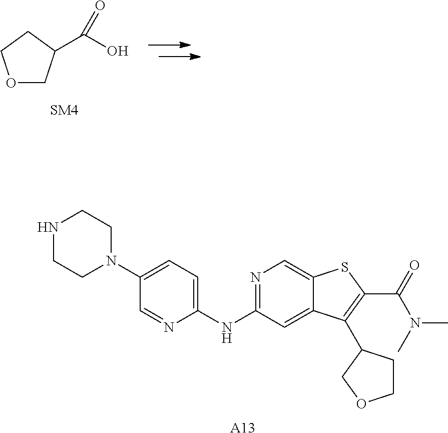 [SMILES: O=C(O)C1CCOC1 ; CN(C)C(=O)c1sc2cnc(Nc3ccc(N4CCNCC4)cn3)cc2c1C1CCOC1 ; "A13"]

Compound A13 was synthesized in the same method as that for compound A1 in Example 1, using tetrahydrofuran-3-carboxylic acid SM4 which is commercially available from PharmaBlock, Nanjing, China. ¹HNMR (400 MHz, DMSO-d₆) δ(ppm) 9.48 (s, 1H), 8.85 (s, 1H), 8.51 (s, 1H), 8.87 (d, 1H), 7.39 (brs, 2H), 4.20-4.35 (m, 1H), 3.96-4.00 (m, 1H), 3.89 (q, 1H), 3.83 (t, 1H), 3.80 (m, 1H), 3.55-3.63 (m, 1H), 2.93-3.04 (m, 14H), 2.28-2.33 (m, 3H). LC-MS(m/z):453 (M+H⁺).

### Example 8: Synthesis of Compound A14

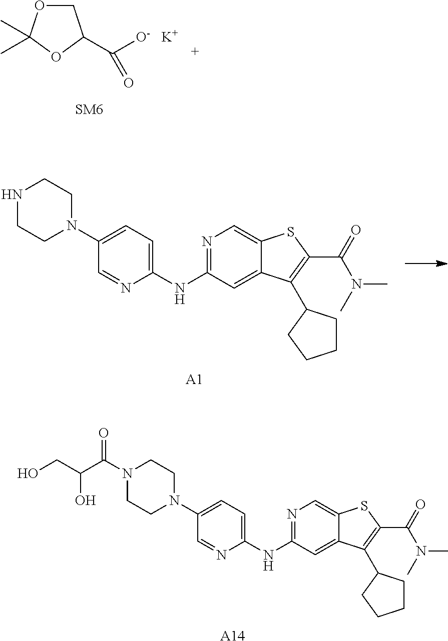

To a stirred suspension of SM6 commercial available from PharmaBlock, Nanjing, China (0.184 g, 1.0 mmol, 1.0 eq.) and compound A1 (0.450 g, 1.0 mmol, 1.0 eq.) was added DIPEA (0.387 g, 3.0 mmol, 3.0 eq.) and 1-[bis(dimethylamino)methylene]-1H-1,2,3-triazolo[4,5-b]pyridinium 3-oxidhexafluorophosphate (HATU, 0.456 g, 1.2 mmol, 1.2 eq.), and the resultant mixture was stirred at ambient temperature for 16 hours. The reaction mixture was poured into water, and was extracted with ethyl acetate. Organic phase was washed with water followed by brine, dried over Na₂SO₄, and was concentrated to dryness.

The obtained crude material was dissolved in dichloromethane (50 ml) was added TFA (10 ml), and the resultant mixture was stirred at ambient temperature for 3 hours. The mixture was concentrated to dryness, and was dissolved in water. The aqueous phase was treated with 3 N NaOH until pH achieved 10, and was extracted with dichloromethane. Organic phase was dried over Na₂SO₄, and was concentrated to dryness. The obtained residue was purified by a flash chromatography (DCM/MeOH=30/1) to afford compound A14 (0.450 g, 84% yield) as a yellow powder. ¹HNMR (400 MHz, DMSO-d₆) δ(ppm) 9.50 (s, 1H), 8.83 (s, 1H), 8.47 (s, 1H), 7.89 (s, 1H), 7.40-7.45 (m, 2H), 4.96 (d, 1H), 4.72 (t, 1H), 4.38 (q, 1H), 3.46-3.70 (m, 6H), 3.20-3.24 (m, 1H), 2.93-3.03 (m, 10H), 1.73-2.01 (m, 8H). LC-MS(m/z): 539 (M+H⁺).

### Example 9: Synthesis of Compounds B1 and B1s

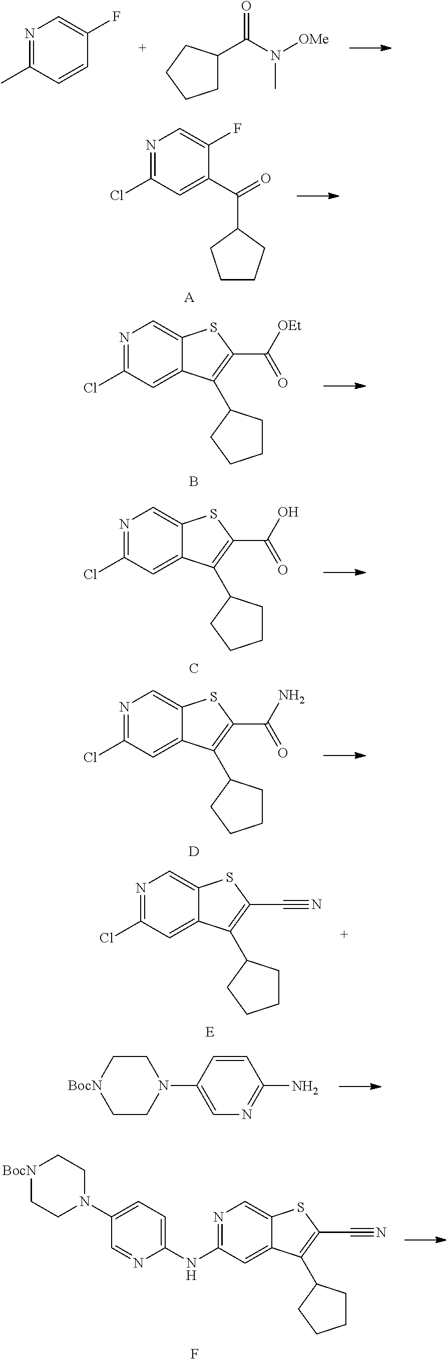

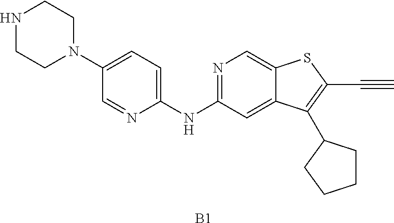 [SMILES: C#Cc1sc2cnc(Nc3ccc(N4CCNCC4)cn3)cc2c1C1CCCC1 ; "B1"]

A solution of 2-chloro-5-fluoropyridine (2.00 g, 19.8 mmol) in dry THF (20 ml) was cooled to −78° C. under nitrogen atmosphere, and a solution of n-BuLi (2.5 M in hexane, 7.3 ml) was added dropwise. The resultant mixture was stirred at the same temperature for additional 1 hour. N-methoxy-N-methyl-cyclopentanecarboxamide (3.11 g, 15.2 mmol) was added dropwise, and the obtained mixture was stirred for additional 0.5 hour before warmed to ambient temperature. The resultant mixture was stirred at ambient temperature for 10 hours, and was quenched with NH₄Cl aqueous solution. The mixture was extracted with EtOAc, washed with brine, dried over MgSO₄, and was concentrated to dryness. The obtained residue was purified by a column chromatography to afford compound A (2.10 g, 61% yield) as a colorless oil.

A solution of compound A (0.50 g, 2.2 mmol) and ethyl thioglycolate (0.32 g, 2.6 mmol) was cooled with an ice-water bath under nitrogen atmosphere, and NaH (60% in oil, 0.18 g, 4.4 mmol) was added in portions. The resultant mixture was heated to reflux for additional 12 hours. The mixture was cooled to ambient temperature, and was poured into cold water. The aqueous phase was extracted with EtOAc, washed with brine, dried over MgSO₄, and was concentrated to dryness. The obtained residue was purified by a column chromatography to afford compound B (0.46 g, 68% yield) as a yellow oil.

To a stirred solution of compound B (0.46 g, 1.5 mmol) in mixed solvent of THF (30 ml) and water (10 ml) was added LiOH (0.5 g), and the mixture was stirred at ambient temperature for 12 hours. The reaction mixture was concentrated to dryness, and was treated with water. The aqueous phase was extracted with DCM, and organic phase was discarded. The aqueous phase was treated with 6 N HCl until pH=2, and precipitate was collected by filtration to afford compound C (0.35 g, 83% yield) as a tan solid.

To a solution of compound C (0.35 g, 1.2 mmol) in dry THF (30 ml) was added CDI (0.23 g, 1.4 mmol), and the obtained mixture was heated to reflux for 3 hours. The mixture was cooled to ambient temperature, and a solution of NH₃ in THF was added. The resultant mixture was stirred at ambient temperature for additional 12 hours. The mixture was concentrated, and was purified by a column chromatography to afford compound D as a crude solid which was used directly in the next step.

The crude compound D was added to POCl₃ (50 ml), and the mixture was heated to reflux for 6 hours. The mixture was cooled to ambient temperature, and was quenched by cold water. The aqueous phase was extracted with EtOAc, washed with brine, dried over MgSO₄, and was concentrated to dryness. The obtained residue was purified by a column chromatography to afford compound E (0.30 g, 90% yield over two steps) as a tan solid.

To a stirred solution of compound E (40 mg, 0.12 mmol) and tert-butyl 4-(6-aminopyridin-3-yl) piperazine-1-carboxylate (40 mg, 0.14 mmol) in 1,4-dioxane (10 ml) was added Cs₂CO₃ (117 mg, 0.36 mmol), catalytic Pd₂(dba)₃ and catalytic Xantphos, and the resultant mixture was heated to reflux under nitrogen atmosphere for 12 hours. The mixture was cooled to ambient temperature, and was concentrated to dryness. The obtained residue was purified by a column chromatography to afford compound F (80 mg, 100% yield) as a yellowish solid.

To a stirred solution of compound F (80 mg, 0.12 mmol) in DCM (10 ml) was added TFA (3 ml), and the resultant mixture was stirred at ambient temperature for 12 hours. The mixture was concentrated to dryness, and was dissolved in DCM. The organic phase was washed with Na₂CO₃ aqueous solution, dried over MgSO₄, and was concentrated to dryness. The obtained residue was treated with MTBE, and was filtered to afford compound B1 (50 mg, 92% yield) as a yellowish solid. ¹HNMR (400 MHz, DMSO-d₆) δ(ppm) 9.64 (s, 1H), 9.00 (d, 1H), 8.65 (s, 1H), 7.88 (d, 1H), 7.40-7.43 (m, 1H), 7.32-7.34 (m, 1H), 3.58-3.62 (m, 1H), 3.01-3.03 (m, 4H), 2.89-2.91 (m, 4H), 2.04-2.14 (m, 8H). LC-MS(m/z): 405 (M+H⁺).

Compound B1s was prepared by adding saturated HCl gas in 1,4-dioxane into a solution of compound B1 in ethanol. The resultant mixture was stirred at ambient temperature for additional 1 hour, and the reaction was concentrated to dryness to afford compound B1s as a yellow solid. ¹HNMR (400 MHz, D₂O) S(ppm) 8.76 (s, 1H), 7.94-7.97 (m, 1H), 7.53 (d, 1H), 7.12-7.19 (m, 2H), 3.43 (m, 9H), 2.07 (m, 2H), 1.74-1.76 (m, 6H). LC-MS(m/z): 405 (M+H⁺).

### Example 10: Synthesis of Compounds B2˜B10

Compounds B2˜B10 were synthesized in the same method as that for compound B1 in Example 9, starting from different anilines. ¹HNMR and MS data of compounds were listed in the following.

Compound B2: ¹HNMR (400 MHz, DMSO-d₆) δ(ppm) 8.90 (brs, 1H), 8.87 (s, 1H), 8.30 (d, 1H), 7.81 (d, 1H), 7.18 (s, 1H), 6.83 (d, 1H), 3.51 (m, 5H), 2.90 (m, 4H), 1.93 (m, 8H). LC-MS(m/z): 405 (M+H⁺).

Compound B3: ¹HNMR (400 MHz, DMSO-d₆) δ(ppm) 8.91 (brs, 1H), 7.43 (d, 8H), 7.23 (d, 1H), 6.92 (d, 2H), 3.53 (m, 1H), 3.09 (m, 8H), 1.99 (m, 8H). LC-MS(m/z): 404 (M+H⁺).

Compound B4: ¹HNMR (400 MHz, DMSO-d₆) δ(ppm) 8.85-8.91 (m, 2H), 7.39 (d, 2H), 7.21 (d, 1H), 6.89 (d, 2H), 3.50-3.55 (m, 1H), 3.05-3.07 (m, 4H), 2.46-2.49 (m, 4H), 2.23 (s, 3H), 1.89-2.09 (m, 8H). LC-MS(m/z): 418 (M+H⁺).

Compound B5: ¹HNMR (400 MHz, DMSO-d₆) δ(ppm) 9.63 (s, 1H), 9.00 (d, 1H), 8.64 (s, 1H), 7.89 (d, 1H), 7.32-7.44 (m, 2H), 6.58-3.62 (m, 1H), 3.07-3.09 (m, 4H), 2.46-2.49 (m, 4H), 2.23 (m, 1H), 2.01-2.13 (m, 8H). LC-MS(m/z): 419 (M+H⁺).

Compound B6: ¹HNMR (400 MHz, DMSO-d₆) δ(ppm) 8.90 (d, 2H), 7.43 (d, 2H), 7.22 (s, 1H), 6.92 (d, 2H), 3.73-3.75 (m, 4H), 3.48-3.55 (m, 1H), 3.02-3.04 (m, 4H), 1.77-2.10 (m, 8H). LC-MS(m/z): 405 (M+H⁺).

Compound B7: ¹HNMR (400 MHz, DMSO-d₆) δ(ppm) 8.89 (d, 2H), 8.32 (d, 1H), 7.82-7.84 (m, 1H), 7.19 (d, 1H), 6.84 (d, 1H), 3.70-3.73 (m, 4H), 3.51-3.56 (m, 1H), 3.45-3.47 (m, 4H), 1.78-2.10 (m, 8H). LC-MS(m/z): 406 (M+H⁺).

Compound B8: ¹HNMR (400 MHz, DMSO-d₆) δ(ppm) 8.90 (d, 2H), 8.61 (s, 2H), 7.20 (s, 1H), 3.60 (m, 9H), 1.98 (m, 8H). LC-MS(m/z): 407 (M+H⁺).

Compound B9: ¹HNMR (400 MHz, DMSO-d₆) δ(ppm) 8.99 (d, 2H), 7.35 (s, 1H), 7.02-7.14 (m, 3H), 6.53 (d, 1H), 8.52-8.57 (m, 1H), 3.10-3.13 (m, 4H), 2.44-2.47 (m, 4H), 1.77-2.11 (m, 8H). LC-MS(m/z): 418 (M+H⁺).

Compound B10: ¹HNMR (400 MHz, DMSO-d₆) δ(ppm) 9.02 (brs, 1H), 8.98 (d, 1H), 7.35 (s, 1H), 7.26 (m, 2H), 7.06 (d, 1H), 6.55 (m, 1H), 3.75 (m, 4H), 3.56 (m, 1H), 3.09 (m, 4H), 1.98 (m, 8H). LC-MS(m/z): 405 (M+H⁺).

### Example 11: Synthesis of Compound B11

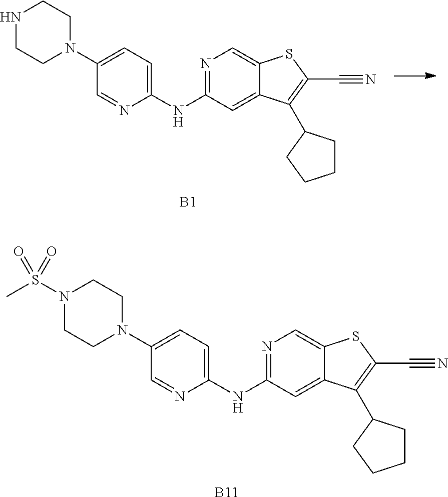 [SMILES: N#Cc1sc2cnc(Nc3ccc(N4CCNCC4)cn3)cc2c1C1CCCC1 ; "B1" ; CS(=O)(=O)N1CCN(c2ccc(Nc3cc4c(C5CCCC5)c(C#N)sc4cn3)nc2)CC1 ; "B11"]

To a stirred solution of compound B1 (40 mg, 0.1 mmol) in dichloromethane (10 ml) was added triethylamine (30 mg, 0.3 mmol), followed by addition of methanesulfonyl chloride (23 mg, 0.2 mmol), and the resultant mixture was stirred at ambient temperature for 16 hours. The reaction mixture was quenched by water, and organic phase was separated. The organic phase was washed with water, dried over MgSO₄, and was concentrated to dryness. The obtained residue was purified by a column chromatography to afford compound B11 (20 mg, 44% yield) as a yellowish solid. ¹HNMR (400 MHz, DMSO-d₆) δ(ppm) 9.69 (s, 1H), 9.01 (s, 1H), 8.65 (s, 1H), 7.94 (d, 1H), 7.48 (m, 1H), 7.37 (m, 1H), 3.62 (m, 1H), 3.20 (m, 8H), 2.94 (s, 3H), 2.05 (m, 8H). LC-MS(m/z): 483 (M+H⁺).

### Example 12: Synthesis of Compound B12

Compound B12 was synthesized in the same method that for compound B11, starting from acyl chlorides instead of sulfonyl chloride. ¹HNMR (400 MHz, DMSO-d₆) δ(ppm) 9.67 (s, 1H), 9.01 (s, 1H), 8.65 (s, 1H), 7.92 (d, 1H), 7.46 (m, 1H), 7.35 (d, 1H), 3.60 (m, 4H), 3.06 (m, 4H), 2.01 (m, 11H). LC-MS(m/z): 447 (M+H⁺).

### Examples 13˜23: Synthesis of Compounds A2˜A5, A8, A9, and A15˜A19

Compounds A2˜A5, A8, A9, and A15˜19 were synthesized in the same method as that for compound A1 in Example 1, starting from corresponding aliphatic carboxylic acids and amines. ¹HNMR and MS data of compounds were listed in the following.

### Example 13

Compound A2: ¹HNMR (400 MHz, DMSO-d₆) δ(ppm) 9.41 (s, 1H), 8.84 (s, 1H), 8.42 (s, 1H), 7.89 (d, 1H), 7.46 (d, 1H), 7.39 (d, 1H), 7.36 (d, 1H), 7.31 (d, 1H), 6.14 (s, 1H), 2.87-3.17 (m, 14H), 2.53-2.57 (m, 2H), 1.96-2.03 (m, 2H). LC-MS(m/z): 449 (M+H⁺).

### Example 14

Compound A3: ¹HNMR (400 MHz, DMSO-d₆) δ(ppm) 10.80 (brs, 1H), 9.02 (s, 1H), 8.89 (brs, 2H), 7.93 (s, 1H), 7.85 (brs, 2H), 7.40-7.43 (d, 1H), 3.06 (s, 3H), 2.96 (s, 3H), 2.30 (s, 3H). LC-MS(m/z): 397 (M+H⁺).

### Example 15

Compound A4: ¹HNMR (400 MHz, DMSO-d₆) δ(ppm) 9.44 (s, 1H), 8.80 (s, 1H), 8.43 (s, 1H), 7.89 (s, 1H), 7.40 (m, 3H), 3.65 (m, 1H), 2.85-3.00 (m, 14H), 2.29-2.41 (m, 4H), 2.10-2.14 (m, 1H), 1.91-1.99 (m, 1H). LC-MS(m/z): 437 (M+H⁺).

### Example 16

Compound A5: ¹HNMR (400 MHz, DMSO-d₆) δ(ppm) 9.44 (s, 1H), 8.81 (s, 1H), 8.50 (s, 1H), 7.89 (s, 1H), 7.48-7.51 (d, 1H), 7.39-7.42 (dd, 1H), 2.93-3.04 (m, 14H), 2.77-2.79 (m, 1H), 1.77-1.86 (m, 7H), 1.30-1.40 (m, 3H). LC-MS(m/z): 465 (M+H⁺).

### Example 17

Compound A8: ¹HNMR (400 MHz, DMSO-d₆) δ(ppm) 9.50 (s, 1H), 8.87 (s, 1H), 8.56 (s, 2H), 7.85 (s, 1H), 7.34-7.41 (m, 2H), 4.28-4.40 (m, 4H), 3.87-3.90 (m, 1H), 3.63-3.68 (m, 1H), 2.92-3.02 (m, 8H), 1.78-2.08 (m, 8H). LC-MS(m/z): 463 (M+H⁺).

### Example 18

Compound A9: ¹HNMR (400 MHz, DMSO-d₆) δ(ppm) 9.54 (s, 1H), 8.86 (s, 1H), 8.53 (s, 1H), 7.90 (d, 1H), 7.39-7.46 (m, 2H), 4.09 (s, 3H), 3.58-3.67 (m, 1H), 3.19-3.20 (m, 4H), 2.26-2.30 (m, 2H), 1.77-2.06 (m, 6H). LC-MS(m/z): 488 (M+H⁺).

### Example 19

Compound A15: ¹HNMR (400 MHz, DMSO-d₆) δ(ppm) 9.43 (s, 1H), 8.82 (s, 1H), 8.43 (s, 1H), 7.89 (s, 1H), 7.45-7.47 (d, 1H), 7.38-7.40 (d, 1H), 3.14-3.21 (m, 1H), 2.85-3.08 (m, 14H), 1.35-1.37 (d, 6H). LC-MS(m/z): 425 (M+H⁺).

### Example 20

Compound A16: ¹HNMR (400 MHz, DMSO-d₆) δ(ppm) 9.41 (s, 1H), 8.80 (s, 1H), 8.38 (s, 1H), 7.90 (s, 1H), 7.36-7.45 (m, 2H), 2.98-3.08 (m, 10H), 2.84-2.86 (m, 4H), 1.90-1.94 (m, 1H), 0.94-0.98 (m, 2H), 0.62-0.65 (m, 2H). LC-MS(m/z): 423 (M+H⁺).

### Example 21

Compound A17: ¹HNMR (400 MHz, DMSO-d₆) δ(ppm) 9.55 (s, 1H), 8.85 (s, 1H), 8.48 (s, 1H), 7.91 (d, 1H), 7.44 (s, 2H), 3.48-3.51 (m, 1H), 3.23-3.28 (m, 8H), 1.73-2.02 (m, 12H). LC-MS(m/z): 477 (M+H⁺).

### Example 22

Compound A18: ¹HNMR (400 MHz, DMSO-d₆) δ(ppm) 9.56 (s, 1H), 8.86 (s, 1H), 8.54 (s, 1H), 7.90 (s, 1H), 7.39-7.44 (m, 2H), 3.75 (s, 4H), 3.58-3.62 (m, 1H), 3.19-3.20 (m, 4H), 1.78-2.06 (m, 8H), 1.25 (s, 6H). LC-MS(m/z): 491 (M+H⁺).

### Example 23

Compound A19: ¹HNMR (400 MHz, DMSO-d₆) δ(ppm) 9.49 (d, 1H), 8.85 (d 1H), 8.54 (d, 1H), 7.90 (d, 1H), 7.18-7.84 (m, 2H), 5.83 (d, 1H), 4.51-4.58 (m, 2H), 4.25-4.29 (m, 2H), 3.74-3.82 (m, 3H), 3.61-3.65 (m, 1H), 3.47-3.58 (m, 4H), 3.17-3.18 (m, 2H), 2.85-2.97 (m, 4H), 1.77-2.07 (m, 8H). LC-MS(m/z): 479 (M+H⁺).

### Example 24: In Vitro CDK Inhibitory Activity

Time Resolved-Fluorescent Resonance Energy Transfer (TR-FRET) was used to determine inhibitory ability of compounds A1˜A19, B1s and B1˜B12 against the enzymatic activity of CDK1, CDK2, CDK4, CDK5, CDK6, CDK7, and CDK9.

Inhibitory activity of compounds against CDK1/2/4/5/6/7 was assessed using Lance TR-FRET assay kits from Perkin Elmer Life Sciences (Waltham, Mass.). CDK1/2/4/5/6/7 enzymes were purchased from ThermoFisher (Waltham, Mass.). 2.5 μL of compound solutions and 2.5 μL of enzyme solutions in kinase assay buffer, which was 50 mM HEPES (pH 7.5) with 1 mM EGTA, 1 mM MgCl₂, 2 mM DTT, and 0.01% Tween-20, were added into a white OptiPlate-384 plate followed by adding 5 μL of fluorescently labeled peptide substrates (Ulight-4E-BP1 for CDK4/6 and Ulight-MBP for CDK1/2/7/9) and ATP mixture solutions. Final concentrations of substrates and DMSO were 100 nM and 0.5%, respectively. Concentrations of ATP and different CDK enzymes were adjusted accordingly to achieve optimal enzymatic activities. Final CDK concentrations were 100, 500, 600, 100, 50, and 200 ng/well and ATP concentrations were 30, 50, 100, 20, 100, and 50 μM for CDK1/2/4/5/6/7, respectively. Assay plates were covered to protect from light and spun for 1 min at 700 RPM. Reactions were allowed to perform for 60 minutes in dark with gentle shaking at room temperature after which 5 μL of 40 mM EDTA in detection buffer was added to stop reactions followed 5 μL of the Eu labeled antibodies giving final concentrations of 2 nM to detect the phosphorylation of the peptide substrates. Assay plates were read with the EnVision™ Multilabel Reader in TR-FRET mode (excitation at 320 nm and emission at 665 nm). The percent of inhibition was calculated from the means of data points using the formula: 100−(Signal_{compound}−NoSignal_{background})*100/(Signal_{vehicle}−NoSignal_{background}). The IC50s were determined with GraphPad Prism by the non-linear sigmoidal curve fitting of data from 10 doses.

The CDK9 kinase inhibitory activity of compounds was tested in the LanthaScreen Eu kinase binding assay from ThermoFisher. LanthaScreen® Eu Kinase binding assays are based on the binding and displacement of a proprietary, Alexa Fluor® 647-labeled, ATP-competitive kinase inhibitor scaffold (kinase tracer) to CDK9. CDK9/cyclin T1, Kinase tracer 236 and LanthaScreen® Eu-anti-His Antibody were purchased form ThermoFisher (Waltham, Mass.). 5 μL of CDK9/cyclin T1 (final 5 nM)/antibody (final 2 nM) mixture, 5 μL of compound in kinase buffer, and 5 μL of tracer (final 30 nM) were added to wells of a white OptiPlate-384 plate in this specific order. Assay plates were covered to protect from light, spun for 1 min at 700 RPM and incubated at room temperature for 1 hour. Assay plates were read with the EnVision™ Multilabel Reader in TR-FRET mode (excitation at 320 nm and emission at 665 nm). The percent of inhibition was calculated from the means of data points using the formula: 100−(Signal_{compound}−NoSignal_{backgound})*100/(Signal_{vehicle}−NoSignal_{background}). The IC50s were determined with GraphPad Prism by the non-linear sigmoidal curve fitting of data from 10 doses.

In vitro CDK inhibitory activity of the compounds of present invention and three reference compounds are tabulated in Table 1. Results in table 1 demonstrate that compounds of the present invention have inhibitory activities (nM) against one or more of CDK kinases selected from the group consisting of CDK1, CDK2, CDK4, CDK5, CDK6, CDK7, and CDK9.

TABLE 1

| | CDK | | | | | | |
|---|---|---|---|---|---|---|---|
| Compound | 1 | 2 | 4 | 5 | 6 | 7 | 9 |
| *Ref 1 | 8438 | 6297 | 3.3 | 2702 | 0.29 | 10400 | 691 |
| †Ref 2 | 9886 | 8201 | 15.3 | inactive | 3.1 | 11190 | 765 |
| [symbol] Ref 3 | 464 | 979 | 3.5 | | 0.6 | 298 | 12.1 |
| A1 | 162 | 346 | 1.2 | 262 | 0.66 | 1102 | 5.3 |
| A2 | | | 5.0 | | 2.3 | | |
| A3 | | | 413 | | 174 | | 420 |
| A4 | | | 4.6 | | 2.1 | | |
| A5 | 39.5 | 942 | 1.4 | 266 | 1.1 | 3291 | 13.4 |
| A6 | 501 | 124 | 3.7 | | 2.1 | | 5.2 |
| A7 | | | 4.7 | | 6.4 | | |
| A8 | | | 31 | | 84 | | 552 |
| A9 | | | 5.3 | | 3.3 | | |
| A10 | | | 262 | | 168 | | 106 |
| A11 | | | 160 | | 50 | | 58 |
| A12 | 3682 | 1111 | 29 | | 20 | | |
| A13 | | | 34 | | 25 | | |
| A14 | 273 | 67.7 | 2.9 | | 2.1 | | 4.2 |
| A15 | | | 20.5 | | 10.7 | | |
| A16 | | | 32.1 | | 21 | | 24 |
| A17 | | | 12 | | 20 | | |
| A18 | | | 60 | | 262 | | |
| A19 | | | 22 | | 18 | | |
| B1 | 5.4 | 2.8 | 1.1 | 24.4 | 0.3 | 14.9 | 1.3 |
| B1s | 8.7 | 9.2 | 0.9 | | 0.3 | 7.2 | 0.6 |
| B2 | 77.4 | 13.3 | 2.4 | | 1.0 | 53.9 | 6.6 |
| B3 | 15.1 | 5.2 | 1.9 | | 0.2 | 31.4 | 1.8 |
| B4 | 31.5 | 10.4 | 1.9 | | 0.7 | 47.0 | 8.6 |
| B5 | 9.1 | 3.3 | 0.7 | | 0.3 | 22.1 | 1.0 |
| B6 | 1683 | 39.6 | 222 | | 19.0 | inactive | 951 |
| B7 | 324 | 19.4 | 61.9 | | 19.4 | inactive | 95.6 |
| B8 | 21950 | 824 | 8659 | | 791 | inactive | 4585 |
| B9 | 170 | 70.8 | 582 | | 120 | 872 | 96.5 |
| B10 | 834 | 308 | 1539 | | 838 | inactive | 308 |
| B11 | 101 | 49.9 | 17.7 | | 5.5 | inactive | 8.1 |
| B12 | 26.2 | 10.3 | 4.1 | | 1.4 | 9792 | 2.5 |

Note: 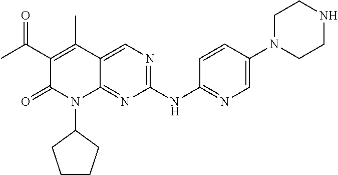 [SMILES: CC(=O)c1c(C)c2cnc(Nc3ccc(N4CCNCC4)cn3)nc2n(C2CCCC2)c1=O]
*Compound “Ref 1” Palbociclib 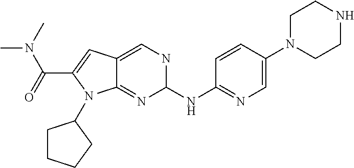 [SMILES: CN(C)C(=O)c1cc2c(n1C1CCCC1)=NC(Nc1ccc(N3CCNCC3)cn1)NC=2]
†Compound “Ref 2” Ricociclib 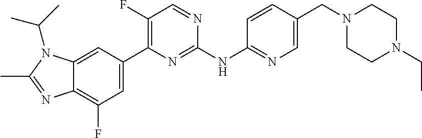 [SMILES: CCN1CCN(Cc2ccc(Nc3ncc(F)c(-c4cc(F)c5nc(C)n(C(C)C)c5c4)n3)nc2)CC1] [symbol]
Compound “Ref 3” Abemaciclib

A disorder may be associated with one single CDK selected from the group consisting of CDK1, CDK2, CDK4, CDK5, CDK6, CDK7, CDK8, and CDK9. For example, disorders associated with CDK1 include breast cancer and prostate cancer. Disorders associated with CDK2 include Glioblastoma Multiforme and Glioblastoma. Disorders associated with CDK4 include Melanoma, Cutaneous Malignant, 3 and CDK4-Related Cutaneous Malignant Melanoma. Disorders associated with CDK5 include Lissencephaly 7 with Cerebellar Hypoplasia and Lissencephaly. Disorders associated with CDK6 include Microcephaly 12, Primary, Autosomal Recessive and Autosomal Recessive Primary Microcephaly. Disorders associated with CDK7 include Breast Cancer. Disorders associated with CDK8 include Colorectal Adenocarcinoma. Disorders associated with CDK9 include HIV-1/AIDS. A disorder may be associated with the combination of 2, 3, 4, 5, 6, or 7 CDKs that are selected from the group consisting of CDK1, CDK2, CDK4, CDK5, CDK6, CDK7, CDK8, and CDK9. A disorder medicated by multiple CDKs may be different from the disorder medicated by each CDK of those multiple CDKs.

Take CDK1 as an example. Encoded by the CDC2 gene, CDK1 is also known as cell division cycle protein 2 homolog, and is a key player in cell cycle regulation. CDK1 is a highly conserved protein that functions as a serine/threonine kinase. CDK1 contains a cleft as the catalytic core in which ATP can fit. Substrates of CDK1 bind near the mouth of the cleft, allowing CDK1 residues to catalyze the covalent bonding of the γ-phosphate to the oxygen of the hydroxyl serine/threonine of the substrate. A T-loop in CDK1 prevents substrate from binding to the active site in the absence of an interacting cyclin. Upon cyclin binding, a PSTAIRE helix in CDK1 moves and rearranges the active site, and facilitates its kinase activities. CDK1 interacts with B-cell lymphoma 2 which is the founding member of the Bcl-2 family of regulator proteins that regulate cell death (apoptosis), by either inducing (pro-apoptotic) or inhibiting (anti-apoptotic) apoptosis. Damage to the Bcl-2 gene has been identified as a cause of a number of cancers, including melanoma, breast, prostate, chronic lymphocytic leukemia, and lung cancer, and a possible cause of schizophrenia and autoimmunity.

Therefore the present invention provides a general method for inhibiting 1, 2, 3, 4, 5, 6 or 7 CDKs selected from CDK1, CDK2, CDK4, CDK5, CDK6, CDK7, and CDK9 comprising contacting the CKD or CDKs with one or more compounds selected from A1˜A19 and B1˜B12, or a pharmaceutically acceptable salt thereof. The present invention further provides specific methods in the following: (1) a method for inhibiting 1 or 2 CDKs selected from CDK4 and CDK6 comprising contacting the CKD or CDKs with one or more compounds selected from A2, A4, A7, A9, A13, A15, A17, A18, and A19, or a pharmaceutically acceptable salt thereof. (2) A method for inhibiting 1, 2 or 3 CDKs selected from CDK4, CDK6, and CDK9 comprising contacting the CKD or CDKs with one or more compounds selected from A3, A8, A10, A11, and A16, or a pharmaceutically acceptable salt thereof. (3) A method for inhibiting 1, 2, 3 or 4 CDKs selected from CDK1, CDK2, CDK4, and CDK6 comprising contacting the CKD or CDKs with compound A12, or a pharmaceutically acceptable salt thereof. (4) A method for inhibiting 1, 2, 3, 4 or 5 CDKs selected from CDK1, CDK2, CDK4, CDK6, and CDK9 comprising contacting the CKD or CDKs with one or more compounds selected from A6, A14, B6, B7, B8, B10, and B11, or a pharmaceutically acceptable salt thereof. (5) A method for inhibiting 1, 2, 3, 4, 5 or 6 CDKs selected from CDK1, CDK2, CDK4, CDK6, CDK7, and CDK9 comprising contacting the CKD or CDKs with one or more compounds selected from B2, B3, B4, B5, B9, and B12, or a pharmaceutically acceptable salt thereof, and B1s. (6) A method for inhibiting 1, 2, 3, 4, 5, 6 or 7 CDKs selected from CDK1, CDK2, CDK4, CDK5, CDK6, CDK7, and CDK9 comprising contacting the CKD or CDKs with one or more compounds selected from A1, A5, and B1, or a pharmaceutically acceptable salt thereof.

Therefore the present invention provides a general method for treating a disease or disorder in a mammal mediated by or associated with 1, 2, 3, 4, 5, 6 or 7 CDKs selected from CDK1, CDK2, CDK4, CDK5, CDK6, CDK7, and CDK9, which method comprises administering to said mammal a therapeutically effective amount of one or more compounds selected from A1˜A19 and B1˜B12, or a pharmaceutically acceptable salt thereof. The present invention further provides specific methods in the following: (1) a method for treating a disease or disorder in a mammal mediated by or associated with 1 or 2 CDKs selected from CDK4 and CDK6, which method comprises administering to said mammal a therapeutically effective amount of one or more compounds selected from A2, A4, A7, A9, A13, A15, A17, A18, and A19, or a pharmaceutically acceptable salt thereof. (2) A method for treating a disease or disorder in a mammal mediated by or associated with 1, 2 or 3 CDKs selected from CDK4, CDK6, and CDK9, which method comprises administering to said mammal a therapeutically effective amount of one or more compounds selected from A3, A8, A10, A11, and A16, or a pharmaceutically acceptable salt thereof. (3) A method for treating a disease or disorder in a mammal mediated by or associated with 1, 2, 3 or 4 CDKs selected from CDK1, CDK2, CDK4, and CDK6, which method comprises administering to said mammal a therapeutically effective amount of compound A12, or a pharmaceutically acceptable salt thereof. (4) A method for treating a disease or disorder in a mammal mediated by or associated with 1, 2, 3, 4 or 5 CDKs selected from CDK1, CDK2, CDK4, CDK6, and CDK9, which method comprises administering to said mammal a therapeutically effective amount of one or more compounds selected from A6, A14, B6, B7, B8, B10, and B11, or a pharmaceutically acceptable salt thereof. (5) A method for treating a disease or disorder in a mammal mediated by or associated with 1, 2, 3, 4, 5 or 6 CDKs selected from CDK1, CDK2, CDK4, CDK6, CDK7, and CDK9, which method comprises administering to said mammal a therapeutically effective amount of one or more compounds selected from B2, B3, B4, B5, B9, and B12, or a pharmaceutically acceptable salt thereof, and B1s. (6) A method for treating a disease or disorder in a mammal mediated by or associated with 1, 2, 3, 4, 5, 6 or 7 CDKs selected from CDK1, CDK2, CDK4, CDK5, CDK6, CDK7, and CDK9, which method comprises administering to said mammal a therapeutically effective amount of one or more compounds selected from A1, A5, and B1, or a pharmaceutically acceptable salt thereof.

In the foregoing specification, embodiments of the present invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicant to be the scope of the invention, is the literal and equivalent scope of the set of claims that issue from this application, in the specific form in which such claims issue, including any subsequent correction.

1. A fused bicyclic compound according to formula (H) or a pharmaceutically acceptable salt thereof: 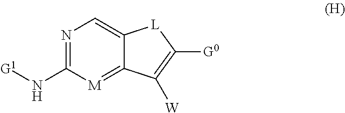
wherein L is —S— or —O—; M is —CH═, —N═ or —CJ=, wherein J is a non-hydrogen monovalent group; and W, G⁰ and G¹ are independently of each other a non-hydrogen monovalent group.

2. The fused bicyclic compound according to claim 1 or a pharmaceutically acceptable salt thereof, wherein L is —S—.

3. The fused bicyclic compound according to claim 1 or a pharmaceutically acceptable salt thereof, wherein M is —CH═.

4. The fused bicyclic compound according to claim 1 or a pharmaceutically acceptable salt thereof, wherein W is C₁₋₆ alkyl, monocyclic C₃₋₁₄ cycloalkyl, C₁₋₆ alkoxy-C₃₋₁₄ cycloalkyl, C₂₋₆ alkynyl, bicyclic C₆₋₁₄ cycloalkyl, heterocycloalkyl, or monocyclic C₃₋₁₄ halocycloalkyl.

5. The fused bicyclic compound according to claim 1 or a pharmaceutically acceptable salt thereof, wherein G¹ has a formula (G^{1a}): 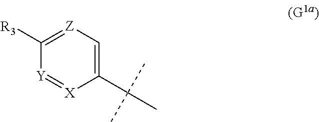
wherein R³ is H or a heterocycloalkyl such as morpholino such as 4-morpholin, 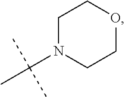
piperazinyl such as piperazin-1-yl 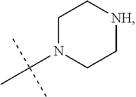
C₁₋₆-alkylpiperazinyl such as 4-methyl-piperazin-1-yl, 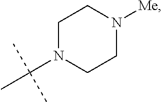
sulfonylpiperazinyl such as 4-(ethylsulfonyl)piperazin-1-yl, 4-(methylsulfonyl)piperazin-1-yl 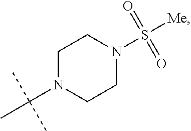
acylpiperazinyl such as acetylpiperazinyl such as 4-acetylpiperazin-1-yl, 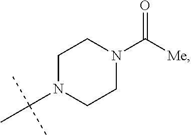
or hydroxylacylpiperazinyl such as 4-(2,3-dihydroxypropanoyl)-piperazin-1-yl 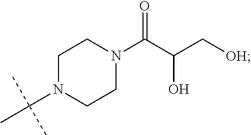
X is CH or N;
Y is N or CR⁴, wherein R⁴ is H or a heterocycloalkyl such as morpholino such as 4-morpholino 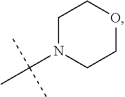
piperazinyl such as piperazin-1-yl, 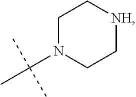
or C₁₋₆alkylpiperazinyl such as 4-methyl-piperazin-1-yl 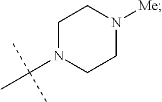
and
Z is CH or N.

6. The fused bicyclic compound according to claim 1 or a pharmaceutically acceptable salt thereof, wherein G¹ has a formula (G^{1b}): 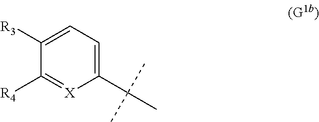
wherein X is CH or N;
R³ is H or a heterocycloalkyl such as morpholino such as 4-morpholin, 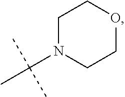
piperazinyl such as piperazin-1-yl, 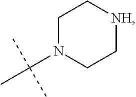
C₁₋₆alkylpiperazinyl such as 4-ethyl-piperazin-1-yl, 4-methyl-piperazin-1-yl 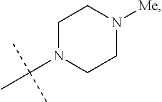
sulfonylpiperazinyl such as 4-(ethylsulfonyl)piperazin-1-yl, 4-(methylsulfonyl)piperazin-1-yl 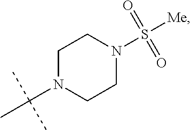
or acylpiperazinyl such as acetylpiperazinyl such as 4-acetylpiperazin-1-yl 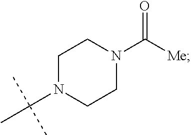
and
R⁴ is H or a heterocycloalkyl such as morpholino such as 4-morpholin 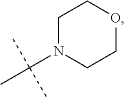
piperazinyl such as piperazin-1-yl 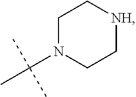
or C₁₋₆alkylpiperazinyl such as 4-methyl-piperazin-1-yl

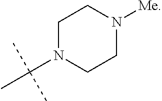

7. The fused bicyclic compound according to claim 1 or a pharmaceutically acceptable salt thereof, wherein G⁰ is cyano —CN; N,N-dialkylaminocarbonyl such as 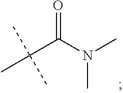
azetidinylcarbonyl such as 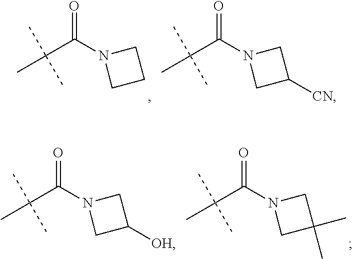
or pyrrolidinylcarbonyl such as 1-pyrrolidinylcarbonyl

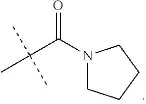

8. The fused bicyclic compound according to claim 1, which is a thieno[2,3-c]pyridine derivative with a Formula (H1), or a pharmaceutically acceptable salt thereof: 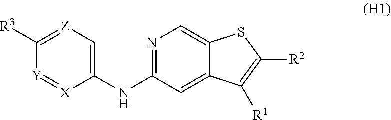
wherein:
X is CH or N;
R¹ is methyl, isopropyl, cyclopropyl, cyclobutyl, cyclopentyl, cyclohexyl, 1-methoxyl cyclopentyl, 1-cyclopentenyl, 3-bicyclo[3.1.0]hexyl, tetrahydrofuran-3-yl, or 3,3-difluorocyclopentyl;
R² is —CN, 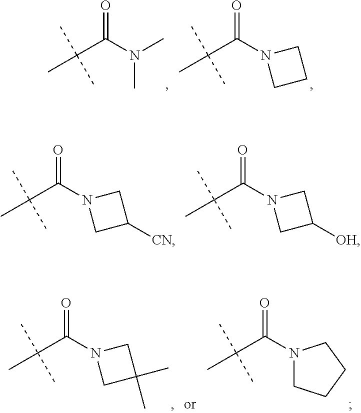
R³ is H, 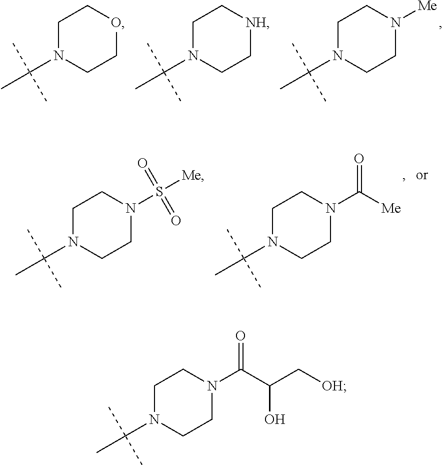
Y is N or CR⁴, wherein R⁴ is H, 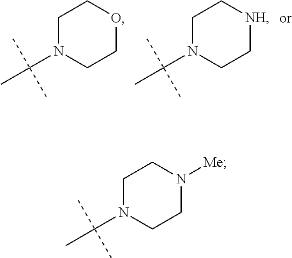
and
Z is CH or N.

9. The fused bicyclic compound according to claim 1, which has Formula (I), or a pharmaceutically acceptable salt thereof: 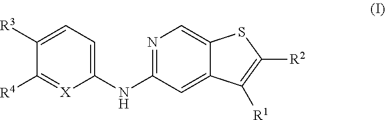
wherein:
X is CH or N;
R¹ is methyl, isopropyl, cyclopropyl, cyclobutyl, cyclopentyl, cyclohexyl, 1-methoxyl cyclopentyl, 1-cyclopentenyl, or 3-bicyclo[3.1.0]hexyl, tetrahydrofuran-3-yl, or 3,3-difluorocyclopentyl;
R² is 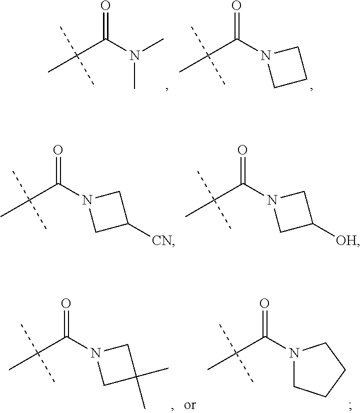
R³ is H, 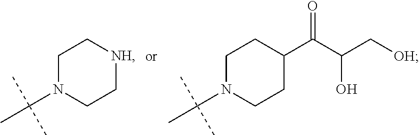
and
R⁴ is H,

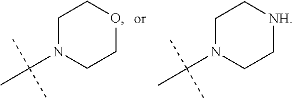

10. The fused bicyclic compound according to claim 9 or a pharmaceutically acceptable salt thereof, wherein X is N, R⁴ is H, and R³ is

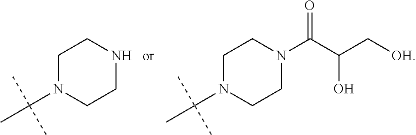

11. The fused bicyclic compound according to claim 9 or a pharmaceutically acceptable salt thereof, wherein X is CH, R³ is H, and R⁴ is

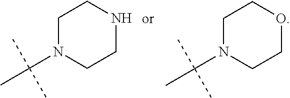

12. The fused bicyclic compound according to claim 1 or a pharmaceutically acceptable salt thereof, wherein the compound is selected from the group consisting of the following compounds:
3-cyclopentyl-N,N-dimethyl-5-((5-(piperazin-1-yl)pyridin-2-yl)amino)thieno[2,3-c]pyridine-2-carboxamide (A1),
3-cyclopentyl-N,N-diethyl-5-((5-(piperazin-1-yl)pyridin-2-yl)amino)thieno[2,3-c]pyridine-2-carboxamide (A1a),
3-cyclopentyl-N-methyl-N-ethyl-5-((5-(piperazin-1-yl)pyridin-2-yl)amino)thieno[2,3-c]pyri dine-2-carboxamide (A1b),
3-(cyclopent-1-en-1-yl)-N,N-dimethyl-5-((5-(piperazin-1-yl)pyridin-2-yl)amino)thieno[2,3-c]pyridine-2-carboxamide (A2),
3-(cyclopent-1-en-3-yl)-N,N-dimethyl-5-((5-(piperazin-1-yl)pyridin-2-yl)amino)thieno[2,3-c]pyridine-2-carboxamide (A2a),
3-(cyclopent-1-en-4-yl)-N,N-dimethyl-5-((5-(piperazin-1-yl)pyridin-2-yl)amino)thieno[2,3-c]pyridine-2-carboxamide (A2b),
3-(cyclopent-1-en-1-yl)-N,N-diethyl-5-((5-(piperazin-1-yl)pyridin-2-yl)amino)thieno[2,3-c]pyridine-2-carboxamide (A2c),
3-(cyclopent-1-en-1-yl)-N-methyl-N-ethyl-5-((5-(piperazin-1-yl)pyridin-2-yl)amino)thieno[2,3-c]pyridine-2-carboxamide (A2d),
N,N,3-trimethyl-5-((5-(piperazin-1-yl)pyridin-2-yl)amino)thieno[2,3-c]pyridine-2-carboxamide (A3),
N,N-dimethyl-3-ethyl-5-((5-(piperazin-1-yl)pyridin-2-yl)amino)thieno[2,3-c]pyridine-2-carboxamide (A3a),
N,N-dimethyl-3-propyl-5-((5-(piperazin-1-yl)pyridin-2-yl)amino)thieno[2,3-c]pyridine-2-carboxamide (A3b),
N,N-dimethyl-3-isopropyl-5-((5-(piperazin-1-yl)pyridin-2-yl)amino)thieno[2,3-c]pyridine-2-carboxamide (A3c),
N,N,-diethyl-3-methyl-5-((5-(piperazin-1-yl)pyridin-2-yl)amino)thieno[2,3-c]pyridine-2-carboxamide (A3d),
N-ethyl-N,3-dimethyl-5-((5-(piperazin-1-yl)pyridin-2-yl)amino)thieno[2,3-c]pyridine-2-carboxamide (A3e),
3-cyclobutyl-N,N-dimethyl-5-((5-(piperazin-1-yl)pyridin-2-yl)amino)thieno[2,3-c]pyridine-2-carboxamide (A4),
3-cyclobutyl-N,N-diethyl-5-((5-(piperazin-1-yl)pyridin-2-yl)amino)thieno[2,3-c]pyridine-2-carboxamide (A4a),
3-cyclobutyl-N-methyl-N-ethyl-5-((5-(piperazin-1-yl)pyridin-2-yl)amino)thieno[2,3-c]pyridine-2-carboxamide (A4b),
3-cyclohexyl-N,N-dimethyl-5-((5-(piperazin-1-yl)pyridin-2-yl)amino)thieno[2,3-c]pyridine-2-carboxamide (A5),
3-cyclohexyl-N,N-diethyl-5-((5-(piperazin-1-yl)pyridin-2-yl)amino)thieno[2, 3-c]pyridine-2-carboxamide (A5a),
3-cyclohexyl-N-methyl-N-ethyl-5-((5-(piperazin-1-yl)pyridin-2-yl)amino)thieno[2,3-c]pyridine-2-carboxamide (A5b),
3-(1-methoxycyclopentyl)-N,N-dimethyl-5-((5-(piperazin-1-yl)pyridin-2-yl)amino)thieno[2,3-c]pyridine-2-carboxamide (A6),
3-(1-ethoxycyclopentyl)-N,N-dimethyl-5-((5-(piperazin-1-yl)pyridin-2-yl)amino)thieno[2,3-c]pyridine-2-carboxamide (A6a),
3-(2-methoxycyclopentyl)-N,N-dimethyl-5-((5-(piperazin-1-yl)pyridin-2-yl)amino)thieno[2,3-c]pyridine-2-carboxamide (A6b),
3-(3-methoxycyclopentyl)-N,N-dimethyl-5-((5-(piperazin-1-yl)pyridin-2-yl)amino)thieno[2,3-c]pyridine-2-carboxamide (A6c),
3-(1-methoxycyclopentyl)-N,N-diethyl-5-((5-(piperazin-1-yl)pyridin-2-yl)amino)thieno[2,3-c]pyridine-2-carboxamide (A6d),
3-(1-methoxycyclopentyl)-N-methyl-N-ethyl-5-((5-(piperazin-1-yl)pyridin-2-yl)amino)thieno[2,3-c]pyridine-2-carboxamide (A6e),
3-(bicyclo[3.1.0]hexan-3-yl)-N,N-dimethyl-5-((5-(piperazin-1-yl)pyridin-2-yl)amino)thieno[2,3-c]pyridine-2-carboxamide (A7),
3-(bicyclo[3.1.0]hexan-3-yl)-N,N-diethyl-5-((5-(piperazin-1-yl)pyridin-2-yl)amino)thieno[2,3-c]pyridine-2-carboxamide (A7a),
3-(bicyclo[3.1.0]hexan-3-yl)-N-methyl-N-ethyl-5-((5-(piperazin-1-yl)pyridin-2-yl)amino)thieno[2,3-c]pyridine-2-carboxamide (A7b),
azetidin-1-yl(3-cyclopentyl-5-((5-(piperazin-1-yl)pyridin-2-yl)amino)thieno[2,3-c]pyridin-2-yl)methanone (A8),
azetidin-2-yl(3-cyclopentyl-5-((5-(piperazin-1-yl)pyridin-2-yl)amino)thieno[2,3-c]pyridin-2-yl)methanone (A8a),
azetidin-3-yl(3-cyclopentyl-5-((5-(piperazin-1-yl)pyridin-2-yl)amino)thieno[2,3-c]pyridin-2-yl)methanone (A8b),
azetidin-1-yl(3-cyclobutyl-5-((5-(piperazin-1-yl)pyridin-2-yl)amino)thieno[2,3-c]pyridin-2-yl)methanone (A8c),
azetidin-1-yl(3-cyclohexyl-5-((5-(piperazin-1-yl)pyridin-2-yl)amino)thieno[2,3-c]pyridin-2-yl)methanone (A8d),
1-(3-cyclopentyl-5-((5-(piperazin-1-yl)pyridin-2-yl)amino)thieno[2,3-c]pyridine-2-carbonyl) azetidine-3-carbonitrile (A9),
1-(3-cyclobutyl-5-((5-(piperazin-1-yl)pyridin-2-yl)amino)thieno[2,3-c]pyridine-2-carbonyl)azetidine-3-carbonitrile (A9a),
1-(3-cyclohexyl-5-((5-(piperazin-1-yl)pyridin-2-yl)amino)thieno[2,3-c]pyridine-2-carbonyl) azetidine-3-carbonitrile (A9b),
3-cyclopentyl-N,N-dimethyl-5-((3-morpholinophenyl)amino)thieno[2,3-c]pyridine-2-carboxamide (A10),
3-cyclobutyl-N,N-dimethyl-5-((3-morpholinophenyl)amino)thieno[2,3-c]pyridine-2-carboxamide (A10a),
3-cyclohexyl-N,N-dimethyl-5-((3-morpholinophenyl)amino)thieno[2,3-c]pyridine-2-carboxamide (A10b),
3-cyclopentyl-N,N-diethyl-5-((3-morpholinophenyl)amino)thieno[2,3-c]pyridine-2-carboxamide (A10c),
3-cyclopentyl-N-methyl-N-ethyl-5-((3-morpholinophenyl)amino)thieno[2,3-c]pyridine-2-carboxamide (A10d),
3-cyclopentyl-N,N-di methyl-5-((3-(piperazin-1-yl)phenyl)amino)thieno[2,3-c]pyridine-2-carboxamide (A11),
3-cyclobutyl-N,N-dimethyl-5-((3-(piperazin-1-yl)phenyl)amino)thieno[2,3-c]pyridine-2-carboxamide (A11a),
3-cyclohexyl-N,N-dimethyl-5-((3-(piperazin-1-yl)phenyl)amino)thieno[2,3-c]pyridine-2-carboxamide (A11b),
3-cyclopentyl-N,N-diethyl-5-((3-(piperazin-1-yl)phenyl)amino)thieno[2,3-c]pyridine-2-carboxamide (A11c),
3-cyclopentyl-N-methyl-N-ethyl-5-((3-(piperazin-1-yl)phenyl)amino)thieno[2,3-c]pyridine-2-carboxamide (A11d),
3-(3,3-difluorocyclopentyl)-N,N-dimethyl-5-((5-(piperazin-1-yl)pyridin-2-yl)amino)thieno[2,3-c]pyridine-2-carboxamide (A12),
3-(3,4-difluorocyclopentyl)-N,N-dimethyl-5-((5-(piperazin-1-yl)pyridin-2-yl)amino)thieno[2,3-c]pyridine-2-carboxamide (A12a),
3-(2,5-difluorocyclopentyl)-N,N-dimethyl-5-((5-(piperazin-1-yl)pyridin-2-yl)amino)thieno[2,3-c]pyridine-2-carboxamide (A12b),
3-(2,2-difluorocyclopentyl)-N,N-dimethyl-5-((5-(piperazin-1-yl)pyridin-2-yl)amino)thieno[2,3-c]pyridine-2-carboxamide (A12c),
3-(2,3-difluorocyclopentyl)-N,N-dimethyl-5-((5-(piperazin-1-yl)pyridin-2-yl)amino)thieno[2,3-c]pyridine-2-carboxamide (A12d),
3-(3,3-dichlorocyclopentyl)-N,N-dimethyl-5-((5-(piperazin-1-yl)pyridin-2-yl)amino)thieno[2,3-c]pyridine-2-carboxamide (A12e),
3-(3,3-difluorocyclopentyl)-N,N-diethyl-5-((5-(piperazin-1-yl)pyridin-2-yl)amino)thieno[2,3-c]pyridine-2-carboxamide (A12f),
3-(3,3-difluorocyclopentyl)-N-methyl-N-ethyl-5-((5-(piperazin-1-yl)pyridin-2-yl)amino)thieno[2,3-c]pyridine-2-carboxamide (A12g),
3-(2,2-difluorocyclobutyl)-N,N-dimethyl-5-((5-(piperazin-1-yl)pyridin-2-yl)amino)thieno[2,3-c]pyridine-2-carboxamide (A12h),
3-(2,2-difluorocyclohexyl)-N,N-dimethyl-5-((5-(piperazin-1-yl)pyridin-2-yl)amino)thieno[2,3-c]pyridine-2-carboxamide (A12i),
3-(3,3-difluorocyclohexyl)-N,N-dimethyl-5-((5-(piperazin-1-yl)pyridin-2-yl)amino)thieno[2,3-c]pyridine-2-carboxamide (A12j),
N,N-dimethyl-5-((5-(piperazin-1-yl)pyridin-2-yl)amino)-3-(tetrahydrofuran-3-yl)thieno[2,3-c]pyridine-2-carboxamide (A13),
N,N-dimethyl-5-((5-(piperazin-1-yl)pyridin-2-yl)amino)-3-(tetrahydrofuran-2-yl)thieno[2,3-c]pyridine-2-carboxamide (A13a),
N,N-diethyl-5-((5-(piperazin-1-yl)pyridin-2-yl)amino)-3-(tetrahydrofuran-3-yl)thieno[2,3-c]pyridine-2-carboxamide (A13b),
N-methyl-N-ethyl-5-((5-(piperazin-1-yl)pyridin-2-yl)amino)-3-(tetrahydrofuran-3-yl)thieno[2,3-c]pyridine-2-carboxamide (A13c),
3-cyclopentyl-5-((5-(4-(2,3-dihydroxypropanoyl)piperazin-1-yl)pyridin-2-yl)amino)-N,N-dimethylthieno[2,3-c]pyridine-2-carboxamide (A14),
3-cyclobutyl-5-((5-(4-(2,3-dihydroxypropanoyl)piperazin-1-yl)pyridin-2-yl)amino)-N,N-dimethylthieno[2,3-c]pyridine-2-carboxamide (A14a),
3-cyclohexyl-5-((5-(4-(2,3-dihydroxypropanoyl)piperazin-1-yl)pyridin-2-yl)amino)-N,N-dimethylthieno[2,3-c]pyridine-2-carboxamide (A14b),
3-cyclopentyl-5-((5-(4-(2,3-dihydroxypropanoyl)piperazin-1-yl)pyridin-2-yl)amino)-N,N-diethylthieno[2,3-c]pyridine-2-carboxamide (A14c),
3-cyclopentyl-5-((5-(4-(2,3-dihydroxypropanoyl)piperazin-1-yl)pyridin-2-yl)amino)-N-methyl-N-ethylthieno[2,3-c]pyridine-2-carboxamide (A14d),
3-isopropyl-N,N-dimethyl-5-((5-(piperazin-1-yl)pyridin-2-yl)amino)thieno[2,3-c]pyridine-2-carboxamide (A15),
3-propyl-N,N-dimethyl-5-((5-(piperazin-1-yl)pyridin-2-yl)amino)thieno[2,3-c]pyridine-2-carboxamide (A15a),
3-n-butyl-N,N-dimethyl-5-((5-(piperazin-1-yl)pyridin-2-yl)amino)thieno[2,3-c]pyridine-2-carboxamide (A15b),
3-isobutyl-N,N-dimethyl-5-((5-(piperazin-1-yl)pyridin-2-yl)amino)thieno[2,3-c]pyridine-2-carboxamide (A15c),
3-isopropyl-N,N-diethyl-5-((5-(piperazin-1-yl)pyridin-2-yl)amino)thieno[2,3-c]pyridine-2-carboxamide (A15d),
3-isopropyl-N-methyl-N-ethyl-5-((5-(piperazin-1-yl)pyridin-2-yl)amino)thieno[2,3-c]pyridine-2-carboxamide (A15e),
3-cyclopropyl-N,N-dimethyl-5-((5-(piperazin-1-yl)pyridin-2-yl)amino)thieno[2,3-c]pyridine-2-carboxamide (A16),
3-cyclopropyl-N,N-diethyl-5-((5-(piperazin-1-yl)pyridin-2-yl)amino)thieno[2,3-c]pyridine-2-carboxamide (A16a),
3-cyclopropyl-N-methyl-N-ethyl-5-((5-(piperazin-1-yl)pyridin-2-yl)amino)thieno[2,3-c]pyridine-2-carboxamide (A16b),
(3-cyclopentyl-5-((5-(piperazin-1-yl)pyridin-2-yl)amino)thieno[2,3-c]pyridin-2-yl)(pyrrolidin-1-yl)methanone (A17),
(3-cyclopentyl-5-((5-(piperazin-1-yl)pyridin-2-yl)amino)thieno[2,3-c]pyridin-2-yl)(pyrrolidin-2-yl)methanone (A17a),
(3-cyclopropyl-5-((5-(piperazin-1-yl)pyridin-2-yl)amino)thieno[2,3-c]pyridin-2-yl)(pyrrolidin-1-yl)methanone (A17b),
(3-cyclobutyl-5-((5-(piperazin-1-yl)pyridin-2-yl)amino)thieno[2,3-c]pyridin-2-yl)(pyrrolidin-1-yl)methanone (A17c),
(3-cyclohexyl-5-((5-(piperazin-1-yl)pyridin-2-yl)amino)thieno[2,3-c]pyridin-2-yl)(pyrrolidin-1-yl)methanone (A17d),
(3-cyclopentyl-5-((5-(piperazin-1-yl)pyridin-2-yl)amino)thieno[2,3-c]pyridin-2-yl)(3,3-dimethylazetidin-1-yl)methanone (A18),
(3-cyclopropyl-5-((5-(piperazin-1-yl)pyridin-2-yl)amino)thieno[2,3-c]pyridin-2-yl)(3,3-dimethylazetidin-1-yl)methanone (A18a),
(3-cyclobutyl-5-((5-(piperazin-1-yl)pyridin-2-yl)amino)thieno[2,3-c]pyridin-2-yl)(3,3-dimethylazetidin-1-yl)methanone (A18b),
(3-cyclohexyl-5-((5-(piperazin-1-yl)pyridin-2-yl)amino)thieno[2,3-c]pyridin-2-yl)(3,3-dimethylazetidin-1-yl)methanone (A18c),
(3-cyclopentyl-5-((5-(piperazin-1-yl)pyridin-2-yl)amino)thieno[2,3-c]pyridin-2-yl)(3,3-diethylazetidin-1-yl)methanone (A18d),
(3-cyclopentyl-5-((5-(piperazin-1-yl)pyridin-2-yl)amino)thieno[2,3-c]pyridin-2-yl)(3-methyl-3-ethyl-azetidin-1-yl)methanone (A18e),
(3-cyclopentyl-5-((5-(piperazin-1-yl)pyridin-2-yl)amino)thieno[2,3-c]pyridin-2-yl)(3-hydroxyazetidin-1-yl)methanone (A19),
(3-cyclopentyl-5-((5-(piperazin-1-yl)pyridin-2-yl)amino)thieno[2,3-c]pyridin-2-yl)(3-hydroxypyrrolidin-1-yl)methanone (A19a),
(3-cyclobutyl-5-((5-(piperazin-1-yl)pyridin-2-yl)amino)thieno[2,3-c]pyridin-2-yl)(3-hydroxy azetidin-1-yl)methanone (A19b),
(3-cyclopropyl-5-((5-(piperazin-1-yl)pyridin-2-yl)amino)thieno[2,3-c]pyridin-2-yl)(3-hydroxyazetidin-1-yl)methanone (A19c), and
(3-cyclohexyl-5-((5-(piperazin-1-yl)pyridin-2-yl)amino)thieno[2, 3-c]pyridin-2-yl)(3-hydroxyazetidin-1-yl)methanone (A19d).

13. The fused bicyclic compound according claim 1 having a Formula (H1), or a pharmaceutically acceptable salt thereof: 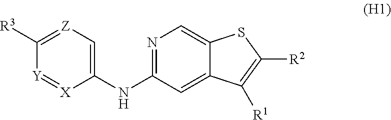
wherein:
X is CH or N;
R¹ is cyclopentyl;
R² is —CN;
R³ is H, 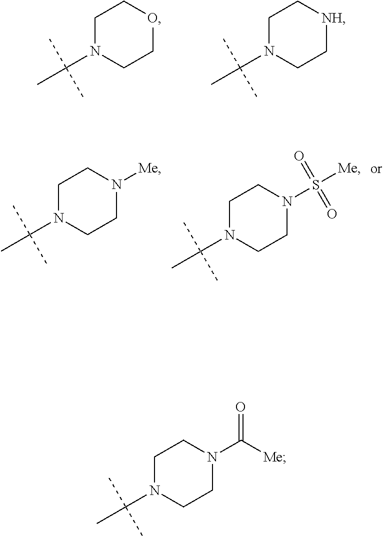
Y is N or CR⁴, wherein R⁴ is H, 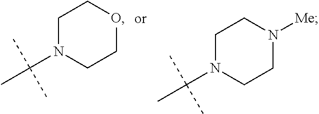
and
Z is CH or N.

14. The fused bicyclic compound according to claim 1 or a pharmaceutically acceptable salt thereof, wherein the compound is selected from the group consisting of the following compounds:
3-cyclopentyl-5-((5-(piperazin-1-yl)pyridin-2-yl)amino)thieno[2,3-c]pyridine-2-carbonitrile (B1),
3-cyclopropyl-5-((5-(piperazin-1-yl)pyridin-2-yl)amino)thieno[2,3-c]pyridine-2-carbonitrile (B1a),
3-cyclobutyl-5-((5-(piperazin-1-yl)pyridin-2-yl)amino)thieno[2,3-c]pyridine-2-carbonitrile (B1b),
3-cyclohexyl-5-((5-(piperazin-1-yl)pyridin-2-yl)amino)thieno[2,3-c]pyridine-2-carbonitrile (B1c),
3-cyclopentyl-5-((6-(piperazin-1-yl)pyridin-3-yl)amino)thieno[2,3-c]pyridine-2-carbonitrile (B2),
3-cyclopentyl-5-((6-(4-methylpiperazin-1-yl)pyridin-3-yl)amino)thieno[2,3-c]pyridine-2-carbonitrile (B2a),
3-cyclopropyl-5-((6-(piperazin-1-yl)pyridin-3-yl)amino)thieno[2,3-c]pyridine-2-carbonitrile (B2b),
3-cyclopropyl-5-((6-(4-methylpiperazin-1-yl)pyridin-3-yl)amino)thieno[2,3-c]pyridine-2-carbonitrile (B2c),
3-cyclobutyl-5-((6-(piperazin-1-yl)pyridin-3-yl)amino)thieno[2,3-c]pyridine-2-carbonitrile (B2d),
3-cyclobutyl-5-((6-(4-methylpiperazin-1-yl)pyridin-3-yl)amino)thieno[2,3-c]pyridine-2-carbonitrile (B2e),
3-cyclohexyl-5-((6-(piperazin-1-yl)pyridin-3-yl)amino)thieno[2,3-c]pyridine-2-carbonitrile (B2f),
3-cyclohexyl-5-((6-(4-methylpiperazin-1-yl)pyridin-3-yl)amino)thieno[2,3-c]pyridine-2-carbonitrile (B2g),
3-cyclopentyl-5-((4-(piperazin-1-yl)phenyl)amino)thieno[2,3-c]pyridine-2-carbonitrile (B3),
3-cyclopropyl-5-((4-(piperazin-1-yl)phenyl)amino)thieno[2,3-c]pyridine-2-carbonitrile (B3a),
3-cyclobutyl-5-((4-(piperazin-1-yl)phenyl)amino)thieno[2,3-c]pyridine-2-carbonitrile (B3b),
3-cyclohexyl-5-((4-(piperazin-1-yl)phenyl)amino)thieno[2,3-c]pyridine-2-carbonitrile (B3c),
3-cyclopentyl-5-((4-(4-methylpiperazin-1-yl)phenyl)amino)thieno[2,3-c]pyridine-2-carbonitrile (B4),
3-cyclopropyl-5-((4-(4-methylpiperazin-1-yl)phenyl)amino)thieno[2,3-c]pyridine-2-carbonitrile (B4a),
3-cyclobutyl-5-((4-(4-methylpiperazin-1-yl)phenyl)amino)thieno[2,3-c]pyridine-2-carbonitrile (B4b),
3-cyclohexyl-5-((4-(4-methylpiperazin-1-yl)phenyl)amino)thieno[2,3-c]pyridine-2-carbonitrile (B4c),
3-cyclopentyl-5-((5-(4-methylpiperazin-1-yl)pyridin-2-yl)amino)thieno[2,3-c]pyridine-2-carbonitrile (B5),
3-cycloproyl-5-((5-(4-methylpiperazin-1-yl)pyridin-2-yl)amino)thieno[2,3-c]pyridine-2-carbonitrile (B5a),
3-cyclobutyl-5-((5-(4-methylpiperazin-1-yl)pyridin-2-yl)amino)thieno[2,3-c]pyridine-2-carbonitrile (B5b),
3-cyclohexyl-5-((5-(4-methylpiperazin-1-yl)pyridin-2-yl)amino)thieno[2,3-c]pyridine-2-carbonitrile (B5c),
3-cyclopentyl-5-((4-morpholinophenyl)amino)thieno[2,3-c]pyridine-2-carbonitrile (B6),
3-cyclopropyl-5-((4-morpholinophenyl)amino)thieno[2,3-c]pyridine-2-carbonitrile (B6a),
3-cyclobutyl-5-((4-morpholinophenyl)amino)thieno[2,3-c]pyridine-2-carbonitrile (B6b),
3-cyclohexyl-5-((4-morpholinophenyl)amino)thieno[2,3-c]pyridine-2-carbonitrile (B6c),
3-cyclopentyl-5-((6-morpholinopyridin-3-yl)amino)thieno[2,3-c]pyridine-2-carbonitrile (B7),
3-cyclopropyl-5-((6-morpholinopyridin-3-yl)amino)thieno[2,3-c]pyridine-2-carbonitrile (B7a),
3-cyclobutyl-5-((6-morpholinopyridin-3-yl)amino)thieno[2,3-c]pyridine-2-carbonitrile (B7b),
3-cyclohexyl-5-((6-morpholinopyridin-3-yl)amino)thieno[2,3-c]pyidine-2-carbonitrile (B7c),
3-cyclopentyl-5-((2-morpholinopyrimidin-5-yl)amino)thieno[2,3-c]pyridine-2-carbonitrile (B8),
3-cyclopropyl-5-((2-morpholinopyrimidin-5-yl)amino)thieno[2,3-c]pyridine-2-carbonitrile (B8a),
3-cyclobutyl-5-((2-morpholinopyrimidin-5-yl)amino)thieno[2,3-c]pyridine-2-carbonitrile (B8b),
3-cyclohexyl-5-((2-morpholinopyrimidin-5-yl)amino)thieno[2,3-c]pyridine-2-carbonitrile (B8c),
3-cyclopentyl-5-((3-(4-methylpiperazin-1-yl)phenyl)amino)thieno[2,3-c]pyridine-2-carbonitrile (B9),
3-cyclopropyl-5-((3-(4-methylpiperazin-1-yl)phenyl)amino)thieno[2,3-c]pyridine-2-carbonitrile (B9a),
3-cyclobutyl-5-((3-(4-methylpiperazin-1-yl)phenyl)amino)thieno[2,3-c]pyridine-2-carbonitrile (B9b),
3-cyclohexyl-5-((3-(4-methylpiperazin-1-yl)phenyl)amino)thieno[2,3-c]pyridine-2-carbonitrile (B9c),
3-cyclopentyl-5-((3-morpholinophenyl)amino)thieno[2,3-c]pyridine-2-carbonitrile (B10),
3-cyclopropyl-5-((3-morpholinophenyl)amino)thieno[2,3-c]pyridine-2-carbonitrile (B10a),
3-cyclobutyl-5-((3-morpholinophenyl)amino)thieno[2,3-c]pyridine-2-carbonitrile (B10b),
3-cyclohexyl-5-((3-morpholinophenyl)amino)thieno[2,3-c]pyridine-2-carbonitrile (B10c),
3-cyclopentyl-5-((5-(4-(methylsulfonyl)piperazin-1-yl)pyridin-2-yl)amino)thieno[2,3-c]pyridine-2-carbonitrile (B11),
3-cyclopropyl-5-((5-(4-(methyl sulfonyl)piperazin-1-yl)pyridin-2-yl)amino)thieno[2,3-c]pyridine-2-carbonitrile (B11a),
3-cyclobutyl-5-((5-(4-(methyl sulfonyl)piperazin-1-yl)pyridin-2-yl)amino)thieno[2,3-c]pyridine-2-carbonitrile (B11b),
3-cyclohexyl-5-((5-(4-(methylsulfonyl)piperazin-1-yl)pyridin-2-yl)amino)thieno[2,3-c]pyridine-2-carbonitrile (B11c),
5-((5-(4-acetylpiperazin-1-yl)pyridin-2-yl)amino)-3-cyclopentylthieno[2,3-c]pyridine-2-carbonitrile (B12),
5-((5-(4-acetylpiperazin-1-yl)pyridin-2-yl)amino)-3-cyclopropylthieno[2,3-c]pyridine-2-carbonitrile (B12a),
5-((5-(4-acetylpiperazin-1-yl)pyridin-2-yl)amino)-3-cyclobutylthieno[2,3-c]pyridine-2-carbonitrile (B12b), and
5-((5-(4-acetylpiperazin-1-yl)pyridin-2-yl)amino)-3-cyclohexylthieno[2,3-c]pyridine-2-carbonitrile (B12c).

15. A pharmaceutical composition comprising a fused bicyclic compound according to claim 1 or a pharmaceutically acceptable salt thereof, and optionally a pharmaceutically acceptable carrier, vehicle or diluent.

16. A method of synthesizing a compound of formula (H1), 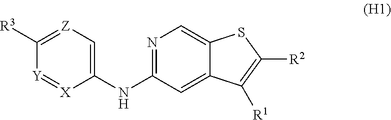
wherein:
X is CH or N;
R¹ is methyl, isopropyl, cyclopropyl, cyclobutyl, cyclopentyl, cyclohexyl, 1-methoxyl cyclopentyl, 1-cyclopentenyl, 3-bicyclo[3.1.0]hexyl, tetrahydrofuran-3-yl, or 3,3-difluorocyclopentyl;
R² is —CN, 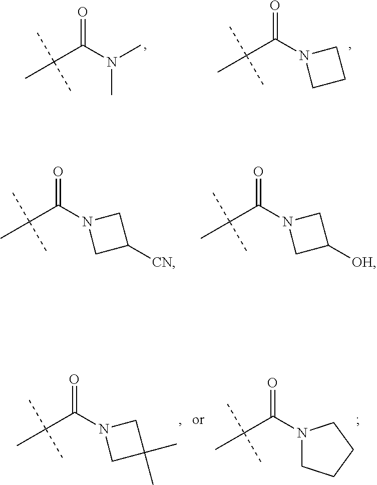
R³ is H, 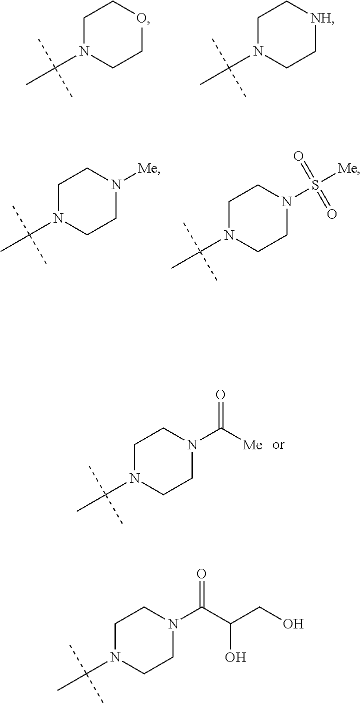
Y is N or CR⁴, wherein R⁴ is H, 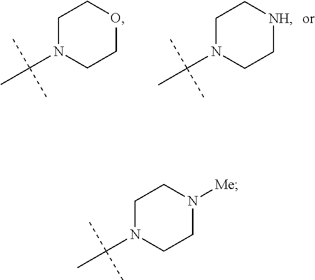
and
Z is CH or N;
the method including:
(1) reacting a compound of formula V1 with a compound of formula V2 to produce compound of formula V3, 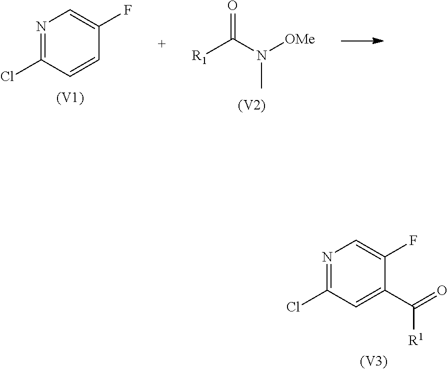
(2) converting the compound of formula V3 to a compound of formula V4, 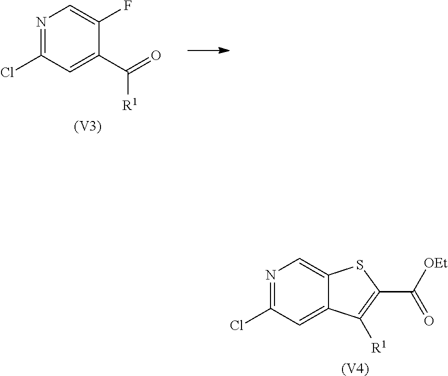
(3) converting the compound of formula V4 to a compound of formula V5, 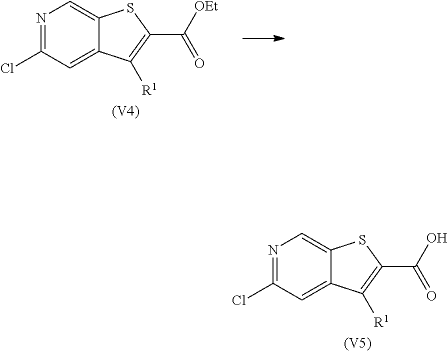
(4) converting the compound of formula V5 to a compound of formula V6, and 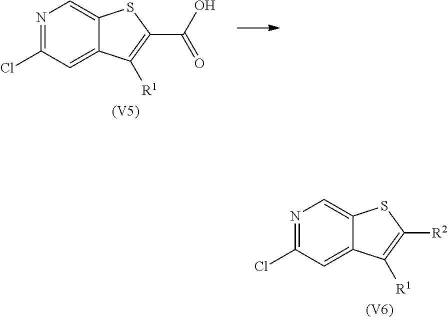
(5) reacting the compound of formula V6 with a compound of formula V7 to produce a compound of formula (H1):

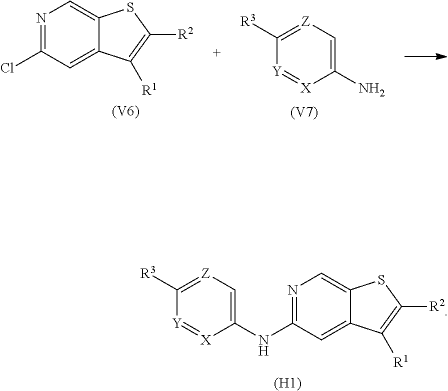

17. A method for treating a CDK-mediated disease or disorder in a mammal, which method comprises administering to said mammal a therapeutically effective amount of a fused bicyclic compound of according to claim 1 or a pharmaceutically acceptable salt thereof.

18. The method according to claim 17, wherein said CDK is CDK1, CDK2, CDK4, CDK5, CDK6, CDK7, CDK9, or any combination thereof.

19. The method according to claim 17, wherein said CDK-mediated disease or disorder is selected from cancer, brain tumor, and autoimmune diseases.

20. A method for inhibiting CDK comprising contacting the CKD with a fused bicyclic compound according to claim 1 or a pharmaceutically acceptable salt thereof.
